(19) United States
(12) Patent Application Publication
(10) Pub. No.: US 2021/0332141 A1
(43) Pub. Date: Oct. 28, 2021

(54) INSULIN-LIKE GROWTH FACTOR-1 RECEPTOR (IGF-1R) BINDING PROTEINS AND METHODS OF USE

(71) Applicants: Pinchas Cohen, Pacific Palisades, CA (US); AMGEN INC., Thousand Oaks, CA (US); Albert Einstein College of Medicine, Bronx, NY (US)

(72) Inventors: Pedro Beltran, Thousand Oaks, CA (US); Derek Huffman, Bronx, NY (US); Nir Brazilal, Bronx, NY (US); Pinchas Cohen, Pacific Palisades, CA (US)

(21) Appl. No.: 16/640,006

(22) PCT Filed: Aug. 30, 2018
(86) PCT No.: PCT/US18/48889

§ 371 Date: Feb. 18, 2020

Related U.S. Application Data
(60) Provisional application No. 62/552,360, filed on Aug. 30, 2017.
(60) Provisional application No. 62/575,937, filed on Oct. 23, 2017.

(51) Int. Cl. C07K 16/28; A61K 45/06; A61K 31/155; A61K 39/395; A61K 31/566; A61P 5/30

(52) U.S. Cl. CPC C07K 16/2863; A61K 45/06; A61K 31/155; A61K 39/3955; A61K 31/566; A61K 2039/55; C07K 2317/565; C07K 2317/515; C07K 2317/51; C07K 2317/76; A61P 5/30

(57) ABSTRACT

The present disclosure provides a method of improvement, preservation, prophylaxis, or inhibition-of-deterioration of a healthspan parameter of a mammalian subject. In exemplary embodiments, the method comprises administering to the subject a composition that comprises an insulin-like growth factor-1 receptor (IGF-1 R) inhibitor, wherein the composition is administered in an amount effective to improve, provide prophylaxis for, or inhibit-the-deterioration of the healthspan parameter.

### CROSS-REFERENCE TO RELATED APPLICATIONS

This application claims priority to U.S. Provisional Application No. 62/552,360, filed on Aug. 30, 2017, and U.S. Provisional Application No. 62/575,937, filed on Oct. 23, 2017. The contents of each application are incorporated herein by reference.

### GRANT FUNDING

This invention was made with government support under Grant Nos. R00AG037574, R56AG052981, P01AG021654, P30AG038072, R37AG018381, R01GM090311, R01 ES020812, P01AG034906, P30AG013319, P30AG050886, P30DK20541, P30CA013330, each of which was awarded by the National Institutes of Health. The government has certain rights in the invention.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 210,555 bytes ASCII (Text) file named “52334A_SeqListing.txt”; created on Aug. 30, 2018.

### BACKGROUND

Diminished growth hormone (GH) and insulin/insulin-like growth factor-1 (IGF-1) signaling extends lifespan in many laboratory models, including mutations to daf2 in worms¹, Sch9 in yeast², and Chico in drosophila³. Likewise, several dwarf models, including Ames, Snell and growth hormone receptor knockout (GHRKO) mice, are exceptionally long-lived⁴⁵. A specific role for IGF-1 receptor (IGF-1R) signaling in the mediation of mammalian longevity was first established in IGF-1R haplo-insufficient mice, which lived 33% longer than controls, but unlike other models of reduced somatotropic signaling, this effect was female specific⁶. This unique sex difference was subsequently confirmed in two follow-up studies, though with more modest improvements in female lifespan^{7,8}, while a life-shortening effect was observed in males⁸. The underlying mechanism(s) linking reduced IGF-1 signaling to improved mammalian lifespan is thought to involve improved stress defenses and lower risk for proliferative diseases⁹⁻¹¹, though the reason for sex differences in this response remains unresolved.

Several examples have also now emerged suggesting the GH/IGF-1 signaling pathway is relevant to human aging¹², including the discovery of functional mutations in the IGF-1R gene in individuals with exceptional longevity, resulting in relative IGF-1 resistance^{13,14}, and in subjects lacking functional GH receptors (Laron dwarfs)¹⁵. Low IGF-1 levels also predict better survival in nonagenarians, and similar to lessons learned in IGF-1R heterozygous mice, this effect is female specific¹⁶. Likewise, higher circulating levels of IGF-1 have been consistently associated with multiple site-specific cancers in epidemiologic studies¹². Thus, given the accumulating evidence across species implicating this pathway as integral to aging and its associated diseases, the development of therapeutics aimed at modulating IGF-1 signaling in humans to mimic or replicate some of the apparent beneficial effects that have been been observed with certain genetic mutation could prove highly effective as a translational tool to delay aging. However, given the constitutive nature of genetic models, and the reported importance of low exposure to GH and IGF-1 early in life on longevity and related outcomes¹⁷, whether benefits can be achieved by targeting this pathway later in life, and in subjects without the genetic blueprint of the models, remains to be determined.

### SUMMARY

The present disclosure provides a method of improvement, preservation, prophylaxis, or inhibition-of-deterioration of a healthspan parameter of a mammalian subject, the method comprising administering to the subject a composition that comprises an insulin-like growth factor-1 receptor (IGF-1R) inhibitor, wherein the composition is administered in an amount effective to improve, provide prophylaxis for, or inhibit-the-deterioration of the healthspan parameter. In some variations, the healthspan parameter is a cardiac health or function, a motor function, a cognitive function, body fatness/leanness, muscle strength, exercise endurance, freedom from malignancy, or an inflammation. In other variations, the healthspan parameter is the modulation of a biomarker associated with longevity or general health, in a direction indicative of health or in a direction opposite which occurs in aging and senescence. Other healthspan parameters will be evident from the detailed description and examples below.

Anti-IGF-1 receptor (IGF-1R) monoclonal antibodies (mAbs), including antibodies which have been developed for treating advanced stage cancers¹⁸⁻²⁰, represent a viable therapeutic tool to target IGF-1 action for new patient populations, including cancer-free populations, and for new indications. We selected such antibodies as a representative class of agents to mimic the beneficial effects associated with diminished IGF-1 signaling on human aging. We developed a mouse mAb against the L2 domain of the IGF-1R (L2-Cmu), which selectively interferes with IGF-1 binding to the murine IGF-1R, in order to enable chronic modulation of this pathway in mice. The work described herein for murine models is intended to be representative of results achievable in other mammalian organisms, including humans. Indeed, the antibody used herein was a murinized version of an antibody that targets human IGF-1R, which represents a class of agents suitable for use in humans.

The present disclosure also provides uses of a composition that comprises an IGF-1R inhibitor for improvement, preservation, prophylaxis, or inhibition-of-deterioration of a healthspan parameter of a mammalian subject, wherein the healthspan parameter is a cardiac health or function, a motor function, a cognitive function, body fatness/leanness, muscle strength, exercise endurance, freedom from malignancy, or an inflammation. Similarly, the disclosure provides uses of a composition that comprises an IGF-1R inhibitor for manufacture of a medicament for the improvement, preservation, prophylaxis, or inhibition-of-deterioration of a healthspan parameter of a mammalian subject, wherein the healthspan parameter is a cardiac health or function, a motor function, a cognitive function, body fatness/leanness, muscle strength, exercise endurance, freedom from malignancy, or an inflammation.

Further provided is an isolated anti-IGF-1R antibody, or an isolated antigen-binding fragment thereof, wherein said antibody or fragment comprises a light chain variable region and a heavy chain variable region, and wherein: (a) said light chain variable region comprises the amino acid sequence of SEQ ID NO:32; and said heavy chain variable region comprises the amino acid sequence of SEQ ID NO:136; or (b) said light chain variable region comprises: (i) the CDR1 sequence of residues 24 through 39 of SEQ ID NO:32; and (ii) the CDR2 sequence of residues 55 through 61 of SEQ ID NO:32; and (iii) the CDR3 sequence of residues 94 through 102 of SEQ ID NO:32; and said heavy chain variable region comprises: (i) the CDR1 sequence of residues 31 through 36 of SEQ ID NO:136; and ii. the CDR2 sequence of residues 51 through 66 of SEQ ID NO:136; and iii. the CDR3 sequence of residues 99 through 108 of SEQ ID NO:136; or (c) said light chain variable region is at least 90 percent identical to SEQ ID NO:32 and said heavy chain variable region is at least 90 percent identical to SEQ ID NO:136, wherein said antibody or fragment comprises a non-human constant region. Related compositions, e.g., pharmaceutical compositions, kits, and articles of manufacture comprising said anti-IGF-1R antibody, or an isolated antigen-binding fragment thereof, are additionally provided herein.

Also contemplated are materials and methods for making IGF-1R inhibitors described herein. For example, the invention includes polynucleotides that encode polypeptide or antibody inhibitors described herein; vectors (including expression vectors) that comprise such polynucleotides; host cells transformed or transfected with such polynucleotides and vectors; and methods of making the polypeptides or antibodies (e.g., by culture of such host cells).

Aspects of the invention that have been described herein as methods also can be described as “uses,” and all such uses are contemplated as aspects of the invention. Likewise, compositions described herein as having a “use” can alternatively be described as processes or methods of using, which are contemplated as aspects of the invention.

Aspects of the invention are described herein as methods of treatment with combinations of two or more agents (or uses of combinations of agents) for a particular purpose. Related aspects of the invention include compositions of matter wherein the two or more agents are in admixture; and kits in which the two or more agents are packaged together, e.g., in unit dose formulations, but not in admixture.

Reference throughout this specification to “one embodiment”, “some embodiments” or “an embodiment” means that a particular feature, structure or characteristic described in connection with the embodiment is included in at least one embodiment of the present disclosure. The particular features, structures, or characteristics described herein may be combined in any suitable manner, and all such combinations are contemplated as aspects of the invention.

Unless otherwise specified, the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

The use of the terms “a” and “an” and “the” and similar referents in the context of describing the invention (especially in the claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

The invention includes, as an additional aspect, all embodiments of the invention narrower in scope in any way than the variations defined by specific paragraphs above or by original claims. For example, where certain aspects of the invention that are described as a genus or set, it should be understood that every member of a genus or set is, individually, an aspect of the invention. Likewise, every individual subset is intended as an aspect of the invention. By way of example, if an aspect of the invention is described as a members selected from the group consisting of 1, 2, 3, and 4, then subgroups (e.g., members selected from {1,2,3} or {1,2,4} or {2,3,4} or {1,2} or {1,3} or {1,4} or {2,3} or {2,4} or {3,4}) are contemplated and each individual species {1} or {2} or {3} or {4} is contemplated as an aspect or variation of the invention. Likewise, if an aspect of the invention is characterized as a range, or being practiced over a range, such as a temperature range, then integer subranges are contemplated as aspects or variations of the invention. Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

The headings herein are for the convenience of the reader and not intended to be limiting. Additional aspects, embodiments, and variations of the invention will be apparent from the Detailed Description and/or Drawing and/or claims. The original claims appended hereto are hereby incorporated by reference as part of the summary of the invention.

Although the Applicant invented the full scope of the invention described herein, the Applicant does not intend to claim subject matter described in the prior art work of others. Therefore, in the event that statutory prior art within the scope of a claim is brought to the attention of the Applicant by a Patent Office, tribunal, or other entity or individual, the Applicant reserves the right to exercise amendment rights under applicable patent laws to redefine the subject matter of such a claim to specifically exclude such statutory prior art or obvious or noninventive variations of statutory prior art from the scope of such a claim. Variations of the invention defined by such amended claims also are intended as aspects of the invention.

### BRIEF DESCRIPTION OF THE DRAWINGS

FIG. 1 demonstrates that antibody L2-Cmu is a selective antagonist to the murine IGF-1R and InsR/IGF-1R hybrids. Panel (a): L2 Domain exhibits the optimal IGF-1R inhibition profile in vitro. Balb/c 3T3 IGF-1R treated with 125 nM antibody alone or antibody plus 5 nm IGF-1 or 20 nm IGF-2 for 5 min. IGF-1R from cell lysates was immunoprecipitated with Biosite D6-12. Blots were probed either with phosphospecific INSR/IGF-1R pY Ab as indicated or C20 Ab recognizing the IGF-1R β-chain. Panels (b-c): Dose response competition assays with murine IGF-1R(ECD)-mFc. The ability of L2-Cmu to block human Ru-labeled IGF-1 or IGF-2 to the murine IGF-1R extracellular domain was evaluated in the IGEN format. Panel (d): NIH-3T3 cells were pre-treated with vehicle or L2-Cmu (100 ug/mL) 1 hr prior to addition of media (Con), or IGF-1 (5 nM) for 2 min to assess activation of IGF-1R and IGF-1R/InsR Hybrids or 15 min later for activation of Akt. L2-Cmu dramatically reduces activation of IGF-1Rs, HybridRs.

FIG. 2 demonstrates that chronic L2-Cmu treatment does not perturb glucose homeostasis in aged mice. (a-d) Glucose and insulin tolerance in 24 mo old female and male mice is not adversely effected by 6 mo mAb treatment (n=12 per group, per sex). (e-h) Chronic mAb treatment led to a non-significant, numerical increase in plasma IGF-1 level in old females (main effect, P=0.097), while no significant difference was observed for IGF-1 levels in male mice (n=8 young, n=15 old old Con and n=16 old mAb per sex), or insulin levels in either sex (n=8 young per sex, n=15 Old Con females, n=16 old Con males, n=16 Old mAb females, n=15 Old mAb males). (i-j) mAb treatment in males and females prevented the age-related increase in hypothalamic IGF-1Rs and reduced cortical levels in males only. (k-l) However, mAb treatment had no effect on InsR levels in examined tissues of either sex (n=8-9 group). Bars represent mean±SE. NS=Not significant. Different letters denote a significant difference between groups, P≤0.05.

FIG. 3 demonstrates that IGF-1R mAb treatment preferentially improves female healthspan. Following 6 mo of mAb treatment, several functional measures of healthspan were assessed in older male and female mice, including exercise tolerance (n=8 Young females, n=17 Old Con females, n=22 Old mAb females; n=8 Young males, n=9 Old Con males, n=9 Old mAb males), grip strength (n=8 Young females, n=12 Old Con females, n=15 Old mAb females; n=8 Young males, n=10 Old Con males, n=9 Old mAb males) and balance beam (n=8 Young females, n=12 Old Con females, n=15 Old mAb females; n=8 Young males, n=9 Old Con males, n=9 Old mAb males). (a-c) In females, mAb led to significant improvements in exercise tolerance (˜50% over old controls), grip strength and motor coordination. (d-f) In males, L2-Cmu improved exercise tolerance by ˜30%, but did not lead to improvements in grip strength, while motor coordination was only modestly improved on a medium difficulty beam (n=8-9 group). Bars represent mean±SE. Different letters denote a significant difference between groups, P≤0.05.

FIG. 4 demonstrates that L2-Cmu mAb treatment prevents age-related diastolic dysfunction in females. (a-d) Cardiac aging in CB6F1 female mice is characterized by a decline in diastolic function (E/A ratio), increased LVPWd, and accumulating amounts of fibrosis and glycerophospholipids in the myocardium. However, mAb treatment, beginning at 78 wks of age, was able to preserve diastolic function, reduce LVPWd measures (Young n=6, Old Con n=8, Old mAb n=7), and largely prevented the accumulation of cardiac fibrosis (Young n=4, Old Con n=5, Old mAb n=5) and glycerophospholipid species (Young n=7, Old Con n=8, Old mAb n=8) in the aged heart. (e-g) Remarkably, the beneficial effects of L2-Cmu on these cardiac parameters (Young n=5, Old Con n=6, Old mAb n=5) and fibrosis (Young n=8, Old Con n=8, Old mAb n=8) were absent in males, suggesting that the beneficial effects of modulating IGF-1 signaling in heart is specific to females. Bars represent mean±SE. Different letters denote a significant difference between groups, P≤0.05. Detailed information regarding the metabolomic data and statistical analysis is provided in Table E6. A colored representation of the data of FIG. 4d has been published as FIG. 4(d) of Mao et al., Nature Communications 9:2394 (2018).

FIG. 5 demonstrates that L2-Cmu mAb treatment mitigates doxorubicin-mediated cardiotoxicity and frailty in older female and male C57BL/6 mice. (a-b) Preemptive mAb treatment in older female C57BL/6 mice improves cardiac resilience to a DOX challenge, and (c) prevented DOX-induced increases in LVIDs. In addition, treatment (i) prevented DOX-induced increases in LVIDs in females, (i-j,l) Likewise, mAb treatment protected against DOX-induced decrements in EF and FS, as well as LVPWd in males. (f,n) Both female and male-treated animals were less frail following the DOX challenge (n=7-9 group). Bars represent mean±SE. NS=Not significant. Different letters denote a significant difference between groups, P<0.05. *Significantly different from Controls, P<0.05.

FIG. 6 demonstrates the sex differences in inflammatory and senescent markers with IGF-1R mAb treatment in aged mice. (a-b) A 25-plex cytokine/chemokine panel was performed on plasma from male and female mice (n=8 young, n=14-16 in older mice, per sex). Data were treated as nonparametric values and analyzed by the Kruskal-Wallis procedure and the Mann-Whitney U test when appropriate. Any value below the lower limit of detection of the assay was replaced by the minimal detectable concentration (MOD)/√2 for the specific analyte. Therefore, undetectable values were treated as a tie for purposes of statistically ranking data. For generation of the above heatmaps, values were normalized against Young Controls and log transformed. Group averages for all analytes are provided in Tables E7-E8. In females, several inflammatory mediators, including IL-6, IL-12p-40, and MIP-1α, were significantly increased with aging, but were largely restored to more youthful levels with mAb treatment. Meanwhile, systemic inflammation in old male mice was markedly exacerbated by mAb treatment, as indicated by a marked increase in the majority of measured analytes over age-matched controls. *P<0.05 versus Old Controls. A colored representation of the data of FIG. 6 (a-b) has been published as FIGS. 5 (a)-(b) of Mao et al., Nature Communications 9:2394 (2018). (c-d) p16 expression in lung, a tissue rich in IGF-1Rs, was reduced by mAb treatment in females, but not in males (n=8-9 group). Bars represent mean±SE. Different letters denote a significant difference between groups, P≤0.05.

FIG. 7 demonstrates that late-life IGF-1R modulation improves female lifespan. (a) Chronic mAb treatment beginning at 78 wks of age (˜18 mo) in older CB6F1 female mice leads to a slight, but significant and persistent reduction in body weight (Group effect, P<0.001). (b-c) This reduction in body weight was attributed to a reduction in lean mass (P<0.05), rather than adiposity, as assessed by qMR. (d) Importantly, late-life mAb treatment was able to significantly improve survival after 78 weeks of age (HR=0.622, P=0.029; n=45 per group), but did not improve maximum lifespan. *Significantly different from Controls, P≤0.05.

FIG. 8 demonstrates the effect of 6 months L2-Cmu mAb treatment on energy balance in aged male and female mice. (a-c) In an initial cohort of older CB6F1 female mice treated for 6 mo with vehicle or mAb, no significant effect was observed on body weight, lean mass or adiposity (n=23-24 per group). (d-f) Likewise, no effect was observed on energy expenditure, or food intake in females, although RER tended to be decreased by mAb treatment (P=0.07). (g-i) In older CB6F1 male mice, L2-Cmu tended to lower body weight and significantly reduced lean mass, without effects on adiposity (n=23-24 per group). (j-l) No effect was observed on energy balance in treated males, but a significant increase in RER was observed with mAb treatment (n=8 per group). Lines and bars are mean±SE. *Significantly different from controls, P≤0.05; #P<0.07 versus Controls

FIG. 9 demonstrates the effect of 6 months L2-Cmu mAb treatment on downstream components of the insulin/IGF-1 signaling pathway in aged female mice (n=8 per group). Bars represent mean±SE. NS=Not significant. Different letters denote a significant difference between groups, P≤0.05.

FIG. 10 demonstrates the effect of 6 months L2-Cmu mAb treatment on downstream components of the insulin/IGF-1 signaling pathway in aged male mice (n=8 per group). Bars represent mean±SE. NS=Not significant. Different letters denote a significant difference between groups, P≤0.05.

FIG. 11 demonstrates the effect of 6 months L2-Cmu mAb treatment on cardiac endpoints in aged female and male mice (n=7-9 per group). Bars represent mean±SE Different letters denote a significant difference between groups, P<0.05.

FIG. 12 demonstrates PCA and PLS plots of metabolites from cardiac tissue in Young Control, Old Control, and Old mAb-treated female mice. Both models isolate Old Control Females from Young Control and Old mAb Females, based on component 1. Component 1 explains most of the variability between samples (b,c). (d) Linear regression was then used to identify metabolite contribution to the variability between samples and PC ae C38:1 was found to be the most dissociative metabolite.

FIG. 13 demonstrates the effect of 6 months L2-Cmu mAb treatment on NF-κB activation in several tissues from Young, Old, and Old mAb treated male and female CB6F1 mice (n=8 per group). Bars represent mean±SE. NS=Not significant. Different letters denote a significant difference between groups, P<0.05.

FIG. 14 demonstrates that L2-Cmu mAb treatment tends to differentially impacts pathology and survival to 24 mo of age in male and female CB6F1 mice. (a, c) Pathologic analysis was performed in an n=16 mice per group, per sex. In females, mAb treatment tended to reduce endometrial hyperplasia but worsen hepatic steotosis, while reducing glomerulonephritis and tending to increase tumor burden in male mice. (b, d) Interim survival was documented in females (n=24 Control males and n=36 mAb females) and in males (n=36 Controls males and n=38 mAb males) to 24 mo old age. Female survival was suggestive of a protective effect with mAb, while male survival with L2C-mu was indistinguishable from controls. Bars represent mean±SE. *Significantly different from Controls, P≤0.05.

### DETAILED DESCRIPTION

Presented herein for the first time are data supporting delayed aging with a therapeutic monoclonal antibody (mAb) via long-term modulation of IGF-1 action. In particular, the data presented herein show that a murinized IGF-1R antibody was feasible and well tolerated in older mammals, and, consistent with genetic models of IGF-1R heterozygosity⁶⁻⁸, improves female healthspan and survival. Therapeutic or prophylactic intervention to delay aging is intended as an aspect or embodiment of the invention, and is an intended effect of interventions described herein for improving healthspan. Advantageously, these effects were achieved even though treatment was not initiated until late stages of life (e.g., ˜18 months of age in mice), suggesting that IGF-1R mAbs represent a readily-available tool to potentially treat at least some manifestations of aging in older humans.

Accordingly, some aspects or embodiments of the invention are a method of improvement, preservation, prophylaxis, or inhibition-of-deterioration of a healthspan parameter of a subject, e.g., a mammalian subject. In exemplary embodiments, the method comprises administering to the subject a composition comprising an insulin-like growth factor-1 receptor (IGF-1R) inhibitor, wherein the composition is administered in an amount effective to improve, provide prophylaxis for, or inhibit-the-deterioration of the healthspan parameter. In exemplary aspects, the healthspan parameter is a cardiac health or function, a motor function, a cognitive function, body fatness/leanness, muscle strength, exercise endurance, freedom from malignancy, or an inflammation.

Compositions described herein, particularly compositions intended for administration and/or prophylaxis and/or therapy, optionally include one or more pharmaceutically acceptable diluents, adjuvants, excipients, carriers, or other formulating agents.

As used herein, the term “improvement” of a parameter (such as a healthspan parameter) refers to a change of a measurable parameter in a direction associated with a beneficial medical, or health effect between measurements of the parameter at successive times. For example, a lowering of blood pressure in a subject considered to be hypertensive, or a lowering of blood glucose in a subject considered to be hyperglycemic, represents an improvement.

As used herein, the term “preservation” of a parameter refers to maintenance of a parameter at a current level, e.g., with no statistically significant change, over a minimum period of time during which deterioration of the parameter is usually measurable. By way of example, cognitive functions and exercise capacity tend to deteriorate or decline in measurable ways in adult human subjects as part of aging. “Preservation” includes the arrest of such decline over a period of time during which decline is usually measurable. Subjects who experience an improvement of the parameter as a result of the intervention can be said to fall within the group of subjects that experience preservation of the parameter. A dose is considered effective to demonstrate “preservation” if the preservation effect is observable over a clinically meaningful time, e.g., 1, 2, 3, 4, 5, 6, 7, 8, 9, 10, 11, 12, 15, 18, 24, 30, 36, 42, 48, 60 or more months, or 1, 2, 3, 4, 5, 6, 7, 8, 9, 10, or more years, even though preservation effects cannot be expected to last in perpetuity.

The term “inhibition of deterioration” of a parameter refers to a slowing or retarding of the deterioration of a parameter, e.g., deterioration at a less than typical level, over a minimum period of time during which deterioration of the parameter is usually measurable in the absence of intervention. By way of example, cognitive functions and exercise capacity tend to decline in measurable ways in an adult human subject as part of aging. If one of these parameters typically deteriorates 20% over period of time “X” in a class of subjects, yet subjects who receive an intervention experience deterioration of only 10% during that period of time, then inhibition of deterioration has occurred in such subjects. Likewise, if the deterioration initially occurs more slowly, e.g., during the the first 25% or 50% of period X, then inhibition of deterioration is demonstrated, even if the subjects ultimately display 20% deterioration at time X. Subjects who experience an improvement of a parameter over a period of time, or who experience preservation, can be said to fall within the group of subjects that experience inhibition of deterioration.

As used herein the term “prophylaxis” means prevention of disease or other undesirable/adverse health event or process. The term “prevent” as well as words stemming therefrom, as used herein, does not necessarily imply 100% or complete prevention. Rather, there are varying degrees of prevention of which one of ordinary skill in the art recognizes as having a potential benefit or therapeutic effect. In this respect, the methods described herein can provide any amount of any level of prevention in a subject. Furthermore, the prevention can include prevention of one or more conditions or symptoms of the disease (e.g., cancer) being prevented. Also, for purposes herein, “prevention” can encompass delaying the onset of the disease, or a symptom or condition thereof. In exemplary aspects, the methods prevent the onset or recurrence of the cancer by 1 day, 2 days, 4 days, 6 days, 8 days, 10 days, 15 days, 30 days, two months, 4 months, 6 months, 1 year, 2 years, 4 years, or more. In exemplary aspects, the methods prevent by way increasing the survival of the subject.

Improvement, preservation, prophylaxis, inhibition-of-deterioration, and prevention are sometimes demonstrable on an individual basis by measuring an indicator, marker, or parameter in question over a minimum clinically meaningful amount of time, which will vary depending on the health assessment in question. Exemplary periods of time include, e.g., 1, 2, 3, 4, 5, 6, 7, 8, 9, 10, 11, 12, 15, 18, 24, 30, 36, 42, 48, 60 or more months, or 1, 2, 3, 4, 5, 6, 7, 8, 9, 10, or more years. Additionally or alternatively, improvement, preservation, prophylaxis, inhibition-of deterioration, and prevention are demonstrable in a population by measuring the parameter in question in the population over time. At the population level, improvement, preservation, prophylaxis, inhibition-of-deterioration, and prevention can be demonstrated statistically, by comparing measurements of a treated population over time with measurements of a control population that did not receive the treatment. While it may not be possible to prove an effect at the individual level for every type of health assessment (e.g., increased lifespan or healthspan), such effects often can be demonstrated on a population level through statistical analysis. A dose that is “effective to” improve, preserve, provide prophylaxis, inhibit-deterioration, or prevent can be estimated or demonstrated with a population study. At least for parameters that are difficult or hard to prove at the individual level, an individual who receives the effective dose, over the period required to demonstrate the effect at the population level, is scored as an individual in whom improvement, preservation, prophylaxis, or inhibition-of-deterioration of the healthspan parameter has been achieved.

Healthspan Parameters

As used herein, the term “healthspan” refers to the length of time that a subject, e.g., a mammalian subject, is healthy. In the context of a population, “healthspan” refers to an average length of time that subjects are healthy. The term “healthspan” is associated with, but a variation of, the term “lifespan,” which refers to the length of an individual's life or the average length of life of a population, healthy or unhealthy. The latter term disregards the health status of a subject, whereas the former term concerns only the time that a subject (or average time that a population) is healthy. The term “healthy” as used herein is meant free from serious disease (especially long term or progressive disease or diseases that more than transiently affect quality of life) or debilitating pain. Accordingly, the term “healthspan parameter” refers to a physical property which characterizes healthspan.

In exemplary aspects, the healthspan parameter relates to lifespan, survival, and/or risk of death. In exemplary aspects, the method of the present disclosure is a method of increasing a subject's lifespan, increasing a subject's survival or decreasing risk of death. In exemplary aspects, the method increases a subject's lifespan or survival by at least or about 5%, relative to untreated controls. In exemplary aspects, the method increases a subject's lifespan or survival by at least or about 10%, relative to untreated controls. In exemplary aspects, the method increases a subject's lifespan or survival by at least or about 15%, relative to untreated controls. In exemplary aspects, the method increases a subject's lifespan or survival by at least or at least or about 1 month, at least or about 2 months, at least or about 3 months, at least or about 4 months, at least or about 5 months, at least or about 6 months, if not longer, relative to untreated controls. Suitable assays for measuring survival and risk of death are known in the art and described here in Example 1.

In exemplary aspects, the healthspan parameter is a motor function, a cognitive function, a cardiac health or function, body fatness/leanness, muscle strength, exercise endurance, freedom from malignancy, or an inflammation.

In exemplary embodiments, the healthspan parameter comprises a motor function. Motor function is any activity or movement which is completed due to the use of motor neurons. In exemplary aspects, the motor function is an activity movement which is completed due to the use of lower motor neurons, or any motor neuron located in the cranial nerves or anterior horn on the spinal cord of humans. In exemplary aspects, the motor function is an activity movement which is completed due to the use of upper motor neurons, or any neuron of the primary motor cortex which aids in the corticospinal tract and impact or modulate the lower motor neurons.

In some exemplary aspects, the motor function comprises a fine motor function, or a coordination of small muscles in movements, such as, for instance, the synchronization of hands and fingers with eyes. Fine motor function is associated with dexterity.

In some exemplary aspects, the motor function comprises a gross motor function. In exemplary instances, the gross motor function comprises limb strength, balance, gait speed, exercise capacity, or coordination.

In exemplary aspects, the subject has been diagnosed with or has self-reported a deterioration in said motor function prior to said administering step. In exemplary instances, the method further comprises a step, prior to the administering step, of screening a motor function of the subject and identifying a motor function deficit, compared to a motor function index, or identifying a motor function deterioration, compared to a measurement of the motor function from prior screening of the subject.

In some aspects, the invention includes combination therapies for motor function. For instance, in some aspects the method further comprises administering to the subject a myostatin inhibitor.

In exemplary aspects, the healthspan parameter is a cognitive function.

In exemplary embodiments, the healthspan parameter comprises a cardiac health or function. For instance, the cardiac health parameter comprises, in some aspects, myocardial fibrosis.

In some aspects, the cardiac function is diastolic function. In exemplary aspects, the diastolic function is a lack of diastolic dysfunction. As used herein, the term “diastolic dysfunction” refers to a condition in which abnormalities in mechanical function are present during diastole. Diastolic dysfunction can occur in the presence or absence of heart failure and can co-exist with or without abnormalities in systolic function (Zile et al., JACC 41: 1519-1522 (2003)). Accordingly, in some embodiments, the diastolic dysfunction is diastolic dysfunction in the absence of systolic dysfunction, which is also known as, diastolic dysfunction with preserved ejection fraction, diastolic dysfunction with preserved systolic function, and diastolic dysfunction with preserved left ventricular function. As used herein, the term “preserved ejection fraction” refers to a left ventricular ejection fraction which is greater than or about 45%, e.g., greater than or about 50%. In some aspects, the preserved ejection fraction is one which is greater than or about 50%. In some embodiments, the diastolic dysfunction is an early diastolic dysfunction. As used herein, the term “early diastolic dysfunction” refers to a medical condition in which ventricle filling is impaired as evidenced by the ratio of the peak velocities of blood across the mitral valve in diastole in early filling, the E wave to that during atrial contraction, the A wave, (E/A ratio)<1 and peak early (E′) and late (A′) mitral annular velocities recorded by conventional pulsed wave Doppler method also <1 (Vasan et al., J Am Coll Cardiol 26:1565-1574 (1995); Xie et al., J Am Coll Cardiol 24:132-139 (1994); Moller et al., J Am Coll Cardiol 35:363-370 (2000)).

In some embodiments, the cardiac function is systolic function. In exemplary aspects, the systolic function is a lack of systolic dysfunction. In simple terms, systolic dysfunction is a condition in which the pump function or contraction of the heart (i.e., systole), fails. Systolic dysfunction may be characterized by a decreased or reduced ejection fraction, e.g., an ejection fraction which is less than 45%, and an increased ventricular end-diastolic pressure and volume. In some aspects, the strength of ventricular contraction is weakened and insufficient for creating an appropriate stroke volume, resulting in less cardiac output.

In some embodiments, the healthspan parameter comprises a cardiac muscle health or function. In some aspects, the subject is at risk of suffering from cardiotoxicity, a condition wherein the heart muscle is damaged and often leads to the heart's inability to pump blood throughout the body. In exemplary aspects, the subject is at risk for cardiotoxicity due to the subject being treated with a chemotherapy drug or other medication which causes cardiotoxicity. In some aspects, the method of the present disclosure protects the subject from developing cardiotoxicity.

In exemplary aspects, the subject has been diagnosed with or has a self-reported a deterioration in said cardiac health or function prior to said administering step. In some aspects, the method further comprises a step, prior to the administering step, of screening a cardiac function of the subject or in a sample from the subject, and identifying a cardiac function deficit, compared to a cardiac function index, or identifying a cardiac function deterioration, compared to a measurement of said cardiac function from prior screening of the subject. In some embodiments, the screening comprises an echocardiogram.

In exemplary aspects, the invention provides a combination therapy related to cardiac health or function. For instance, in some variations, the method further comprises administering to the subject a statin, a beta blocker, an ACE inhibitor, or an inotropic agent.

In exemplary aspects, the subject has been diagnosed with heart failure prior to said administering step.

Heart failure (HF) is defined as the ability of the heart to supply sufficient blood flow to meet the body's needs. In some embodiments, the signs and symptoms of heart failure include dyspnea (e.g., orthopnea, paroxysmal nocturnal dyspnea), coughing, cardiac asthma, wheezing, dizziness, confusion, cool extremities at rest, chronic venous congestion, ankle swelling, peripheral edema or anasarca, nocturia, ascites, heptomegaly, jaundice, coagulopathy, fatigue, exercise intolerance, jugular venous distension, pulmonary rales, peripheral edema, pulmonary vascular redistribution, interstitial edema, pleural effusions, or a combination thereof. In some embodiments, the signs and symptoms of heart failure include dyspnea (e.g., orthopnea, paroxysmal nocturnal dyspnea), fatigue, exercise intolerance, jugular venous distension, pulmonary rales, peripheral edema, pulmonary vascular redistribution, interstitial edema, pleural effusions, or a combination thereof. In some embodiments, the symptom of heart failure is one of the symptoms listed in the following table, which provides a basis for classification of heart failure according to the New York Heart Association (NYHA).

| NYHA | |
|---|---|
| Class | Symptoms |
| I | No symptoms and no limitation in ordinary physical |
| | activity, e.g. shortness of breath when walking, |
| | climbing stairs etc. |
| II | Mild symptoms (mild shortness of breath and/or angina) |
| | and slight limitation during ordinary activity. |
| III | Marked limitation in activity due to symptoms, even |
| | during less-than-ordinary activity, e.g. walking short |
| | distances (20-100 m). Comfortable only at rest. |
| IV | Severe limitations. Experiences symptoms even while at |
| | rest. Mostly bedbound patients. |

Patients presenting with signs and/or symptoms of heart failure may be suffering from systolic dysfunction, diastolic dysfunction, or a combination of the two. Heart failure with preserved ejection fraction, which is also known as, heart failure with preserved systolic function, heart failure without systolic dysfunction, and heart failure with preserved left ventricular function, is a clinical condition in which the subject exhibits a preserved ejection fraction (e.g., an ejection fraction which is greater than or about 45%, or greater than or about 50%) along with signs and/or symptoms of heart failure. In some embodiments, the heart failure is acute heart failure with preserved ejection fraction. In some embodiments, the heart failure is chronic heart failure with preserved ejection fraction. In some embodiments, the heart failure is acute and chronic heart failure with preserved ejection fraction. In some embodiments, the heart failure which is diagnosed is a Class I, Class II, Class III, or Class IV heart failure as defined by the New York Heart Association (NYHA). See, for example, The Criteria Committee of the New York Heart Association. Nomenclature and Criteria for Diagnosis of Diseases of the Heart and Great Vessels. 9th ed. Boston, Mass.: Little, Brown & Co; 1994:253-256, and the table above. In some embodiments, the heart failure is an NYHA Class I or Class II heart failure.

In exemplary embodiments the healthspan parameter comprises an inflammation. For instance, the inflammation may comprise or be manifested or measurable as elevated C-reactive protein (CRP), interleukin-6 (IL-6), interleukin-1 (IL-1), interleukin-4 (IL-4), interleukin-5 (IL-5), CXCL-1, interleukin-12p40 (IL-12p40), macrophage inflammatory protein 1α (MIP1α), macrophage inflammatory protein 2 (MIP-2), and/or tumor necrosis factor-alpha (TNFα), in some aspects.

In exemplary embodiments the healthspan parameter comprises an inflammation. For instance, the inflammation may comprise or be manifested or measurable as elevated C-reactive protein (CRP), interleukin-6 (IL-6), interleukin-1 (IL-1), and/or tumor necrosis factor-alpha (TNFα), in some aspects. In exemplary aspects, the inflammation is characterized by an increased erythrocyte sedimentation rate (ESR) and/or an increased plasma viscosity.

Chronic inflammation is reported to contribute to numerous diseases including allergy, arthritis, asthma, atherosclerosis, autoimmune diseases, diabetes, and cancer, and to conditions of aging.

In exemplary aspects, the subject has been diagnosed with or has a self-reported elevated marker of said inflammation prior to said administering step. In exemplary instances, the method further comprises a step, prior to the administering step, of screening a sample from the subject and identifying an elevated inflammatory marker, compared to an index for said marker, or identifying an increase in said inflammatory marker, compared to a measurement from prior screening of the subject. Exemplary samples include tissue samples and fluid samples, such as blood or serum or plasma or cerebrospinal fluid or synovial fluid. Exemplary assays for measuring markers of inflammation include immunoassays.

Combination anti-inflammatory therapies are contemplated. In exemplary instances, the method further comprises administering to the subject a cyclooxygenase inhibitor, a platelet aggregation inhibitor, a statin, a beta-adrenoreceptor antagonist, an immunomodulating drug, or an angiotensin converting enzyme (ACE) inhibitor.

In exemplary embodiments, the healthspan parameter comprises body weight and/or body fatness/leanness. The composition is administered in an amount effective to reduce body weight, decrease body fat, or increase leanness of the subject. In exemplary aspects, the composition is administered in an amount effective to prevent or reduce weight gain, induce or increase weight loss, reduce appetite, decrease food intake, lower the levels of fat in the patient, or decrease the rate of movement of food through the gastrointestinal system. In exemplary aspects, the composition is administered in an amount effective to improve muscle quality.

In exemplary aspects, the subject has been diagnosed with or has a self-reported elevated weight or body mass index or percentage of body fat prior to said administering step.

In exemplary instances, the method comprises a step, prior to the administering step, of screening the subject and identifying an elevated weight or body mass index of body fat, compared to an index for said marker, or identifying an increase in weight, body mass index, or body fat, compared to measurement from a prior screening of the subject.

In exemplary aspects, the method is a combination therapy for this healthspan parameter. For example, the method comprises administering to the subject an appetite suppressant or an anti-obesity agent or metformin. Anti-obesity agents known in the art or under investigation include appetite suppressants, including phenethylamine type stimulants, phentermine (optionally with fenfluramine or dexfenfluramine), diethylpropion (Tenuate®), phendimetrazine (Prelu-2®, Bontril®), benzphetamine (Didrex®), sibutramine (Meridia®, Reductil®); rimonabant (Acomplia®), other cannabinoid receptor antagonists; oxyntomodulin; fluoxetine hydrochloride (Prozac); Qnexa (topiramate and phentermine), Excalia (bupropion and zonisamide) or Contrave (bupropion and naltrexone); or lipase inhibitors, similar to XENICAL (Orlistat) or Cetilistat (also known as ATL-962), or GT 389-255. At least two anti-diabetic agents have reportedly caused weight loss in humans with type 2 diabetes. Thus, in exemplary aspects, the method comprises administering to the subject an anti-diabetic agent. Anti-diabetic agents known in the art or under investigation include insulin, leptin, Peptide YY (PYY), Pancreatic Peptide (PP), fibroblast growth factor 21 (FGF21), Y2Y4 receptor agonists, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARγ inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; glucokinase activators (GKA); glucagon receptor antagonists (GRA); or FBPase (fructose 1,6-bisphosphatase) inhibitors.

Patient Population

In exemplary embodiments, the subject is a mammal, i.e., a mammalian subject. As used herein, the term “mammal” refers to any vertebrate animal of the mammalia class, including, but not limited to, any of the monotreme, marsupial, and placental taxa. In some embodiments, the mammal is one of the mammals of the order Rodentia, such as mice and hamsters, and mammals of the order Logomorpha, such as rabbits. In exemplary embodiments, the mammals are from the order Carnivora, including Felines (cats) and Canines (dogs). In exemplary embodiments, the mammals are from the order Artiodactyla, including Bovines (cows) and S wines (pigs) or of the order Perssodactyla, including Equines (horses). In some instances, the mammals are of the order Primates, Ceboids, or Simoids (monkeys) or of the order Anthropoids (humans and apes). In certain aspects, the subject is a bovine, an equine, a feline, a canine, or a primate.

In particular embodiments, the mammal is a human. In exemplary aspects, the subject is female, e.g., a human female. In exemplary aspects, the subject is a menopausal or post-menopausal female. In alternative aspects, the subject is a male. In exemplary aspects, the human subject (e.g., male or female human) is at least 30 years old, at least 35 years old, at least 40 years old, at least 45 years old, at least 50 years old, at least 55 years old, at least 60 years old, at least 65 years old, at least 70 years old, at least 75 years old, or at least 80 years old. In exemplary aspects, the subject is a human female at least about 55 years old.

In exemplary instances, the subject has experienced an age-related decline of a growth hormone, growth hormone releasing hormone (GHRH), or IGF-1. In exemplary aspects, the age-related decline is a decline of IGF-1 below normal serum levels of IGF-1. In exemplary aspects, the age-related decline is a decline of IGF-1 to about 86 μg/L or lower. Methods of measuring such hormones (e.g., IGF-1) in serum obtained from subjects are known in the art. See, e.g., Rosario, Arq Bras Endocrinol Metab 54(5): 477-481 (2010) and Glynn and Agha, International Journal of Endocrinology Aritice ID 972617, (2012). In exemplary aspects, GH is measured via a radioimmunoassay. In exemplary instances, IGF-1 is measured via a immunochemiluminescent assay, such as the Immulite 2000 (Diagnostic Products Corp., Los Angeles, Calif.). In exemplary aspects, GH may be measured via an GHRH+arginine test. Such assays are briefly described here in Example 2.

In exemplary instances, the subject is experiencing or has experienced somatopause. In exemplary instances, the subject is experiencing or has experienced somatopause as determined by an age-related decline of a growth hormone, growth hormone releasing hormone (GHRH), or IGF-1, as described above. In exemplary instances, the subject is about 50 years old.

In exemplary instances, the method comprises a step, prior to the administering step, of diagnosing somatopause through hormone pulsatility measurement. See, e.g., Veldhuis et al., “Motivations and Methods for Analyzing Pulsatile Hormone Secretion, Endocr. Rev. 2008 December; 29(7): 823-864, incorporated herein by reference in its entirety and specifically for its descriptions of pulsatility measurement. Hormone pulsatility measurements may be determined by methods known in the art. See, e.g., Glynn and Agha (2012), supra.

In exemplary aspects, the subject is an adult free of diagnosed or self-reported malignancy (cancer). In exemplary instances, the method further comprises a step, prior to the administering step, of screening the subject or a medical record of the subject for malignancies, and failing to detect or diagnose any malignancies. In exemplary aspects, the subject is a menopausal or post-menopausal female free of diagnosed or self-reported malignancy (cancer). For purposes herein, the malignancy (cancer) of the methods disclosed herein can be any cancer, e.g., any malignant growth or tumor caused by abnormal and uncontrolled cell division that may spread to other parts of the body through the lymphatic system or the blood stream. The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In exemplary aspects, the subject is free of any one or more of: a neoplasm, metastases, or a solid tumor, e.g., a refractory solid tumor, an advanced solid tumor, e.g., PIK3CA mutated advanced solid tumor, PIK3CA amplified advanced solid tumor, advanced refractory solid tumors, lung cancer, small cell lung cancer, non-small cell lung cancer (NSCLC), e.g., advanced squamous NSCLC, extensive stage small cell lung cancer (SCLC), Ewing sarcoma, e.g., metastatic Ewing sarcoma, Ewing's family tumor, estraosseous Ewing's tumor, desmoplastic small round cell tumor, Askin's tumor, primitive neuroectodermal tumor (PNET), metastatic malignant neoplasm in the bone or bone marrow or lung, metastatic peripheral primitive neuroectodermal tumor of bone, peripheral primitive neuroectodermal tumor of soft tissue, rhabdomyosarcoma, e.g., (relapsed or refractory) alveolar or embryonal rhabdomyosarcoma, pancreatic carcinoma, e.g., metastatic pancreatic adenocarcinoma, melanoma, e.g., BRAF mutated melanoma, colorectal cancer, e.g., carcinoma, e.g., Kirsten Rat Sarcoma Virus Oncology Homolog (KRAS) metastaic colorectal cancer, colon cancer, colorectal cancer, gastrointestinal cancer, metastatic colorectal cancer, rectal cancer, KRAS-mutant metastatic colorectal carcinoma, advanced carcinoid tumor, pancreatic neuroendocrine tumors, advanced pancreatic cancer, locally advanced unresectable adenocarcinoma of the pancreas, lymphoma, e.g., non-Hodgkin lymphoma, sarcoma, bone metastases, endocrine cancer, ovarian cancer, e.g., recurrent platinum-sensitive ovarian cancer, ovarian neoplasm, epithelial ovarian cancer, optimally debulked epithelial ovarian cancer, breast cancer, e.g., metastatic breast cancer (MBC), HER-2 overexpressing MBC, hormone-receptor positive locally advanced or metastatic breast cancer, prostate cancer, and small intestine cancer.

In exemplary aspects, the subject is a menopausal or post-menopausal female free of any one or more of: a neoplasm, metastases, or a solid tumor, e.g., a refractory solid tumor, an advanced solid tumor, e.g., PIK3CA mutated advanced solid tumor, PIK3CA amplified advanced solid tumor, advanced refractory solid tumors, lung cancer, small cell lung cancer, non-small cell lung cancer (NSCLC), e.g., advanced squamous NSCLC, extensive stage small cell lung cancer (SCLC), Ewing sarcoma, e.g., metastaic Ewing sarcoma, Ewing's family tumor, estraosseous Ewing's tumor, desmoplastic small round cell tumor, Askin's tumor, primitive neuroectodermal tumor (PNET), metastatic malignant neoplasm in the bone or bone marrow or lung, metastatic peripheral primitive neuroectodermal tumor of bone, peripheral primitive neuroectodermal tumor of soft tissue, rhabdomyosarcoma, e.g., (relapsed or refractory) alveolar or embryonal rhabdomyosarcoma, pancreatic carcinoma, e.g., metastatic pancreatic adenocarcinoma, melanoma, e.g., BRAF mutated melanoma, colorectal cancer, e.g., carcinoma, e.g., Kirsten Rat Sarcoma Virus Oncology Homolog (KRAS) metastaic colorectal cancer, colon cancer, colorectal cancer, gastrointestinal cancer, metastatic colorectal cancer, rectal cancer, KRAS-mutant metastatic colorectal carcinoma, advanced carcinoid tumor, pancreatic neuroendocrine tumors, advanced pancreatic cancer, locally advanced unresectable adenocarcinoma of the pancreas, lymphoma, e.g., non-Hodgkin lymphoma, sarcoma, bone metastases, endocrine cancer, ovarian cancer, e.g., recurrent platinum-sensitive ovarian cancer, ovarian neoplasm, epithelial ovarian cancer, optimally debulked epithelial ovarian cancer, breast cancer, e.g., metastatic breast cancer (MBC), HER-2 overexpressing MBC, hormone-receptor positive locally advanced or metastatic breast cancer, prostate cancer, and small intestine cancer.

In exemplary aspects, the subject is not taking any anti-cancer treatments. In exemplary aspects, the subject is not taking any one or more of: everolimus (RAD001), dasatinib, MEK162, bevacizumab, sorafenib, panitumumab, erlotinib, gemcitabine, BYL719, radiation therapy, e.g., 3-dimensional conformal radiation therapy, external beam radiation therapy, capecitabine, rilotumumab, cyclophosphamide, doxorubicin, etoposide, ifosfamide, vincristine sulfate, conatumumab, FOLFOX6, bevacizumab, fluorouracil, irinotecan hydrochloride, leucovorin calcium, oxaliplatin, trastuzumab, AMG655, paclitaxel, carboplatin, AMG102, etoposide, cisplatin, FOLFIRI, fulvestrant, exemestane, AMG386, metformin, MK-2206, T-DM1, ganetespib, ABT-888, PLX3397, pembrolizumab, talazoparib, patritumab.

In some embodiments, the subject has never been diagnosed with any of the foregoing cancers.

IGF-1R

IGF-1R is a transmembrane receptor tyrosine kinase (Blume-Jensen et al., 2001, Nature 411:355-65). The human IGF-1R is synthesized as a 1367 amino acid precursor polypeptide that includes a 30 amino acid signal peptide removed during translocation into the endoplasmic reticulum (Swiss-Prot: P08069). The IGF-1R proreceptor is glycosylated and cleaved by a protease at positions 708-711 (counting from the first amino acid following the signal peptide sequence) during maturation in the ER-golgi resulting in the formation of an α-chain (1-707) and a β-chain (712-1337) that remain linked by disulfide bonds (Bhaumick et al., 1981, Proc Natl Acad Sci USA 78:4279-83, Chernausek et al., 1981, Biochemistry 20:7345-50, Jacobs et al., 1983, Proc Natl Acad Sci USA 80:1228-31, LeBon et al., 1986, J Biol Chem 261:7685-89, Elleman, et al., 2000, Biochem J 347:771-79). The predominant form of the IGF-1R (and INSR) that exists on the cell-surface is a proteolytically processed and glycosylated (αβ)₂ dimer joined covalently by one or more disulfide bonds.

The extracellular portion of the IGF-1R consists of the α-chain and 191 amino acids of the β-chain (712-905). The receptor contains a single transmembrane spanning sequence (906-929) and a 408-residue cytoplasmic domain that includes a functional tyrosine kinase (Rubin et al., 1983, Nature 305:438-440). Comparative sequence analysis has revealed that the IGF-1R is composed of 11 distinct structural motifs (reviewed by Adams et al., 2000, Cell Mol Life Sci 57:1050-93, Marino-Buslje et al., 1998, FEBS Ltrs 441:331-36, Ward et al., 2001, BMC Bioinformatics 2:4). The N-terminal half of the extracellular domain contains two homologous domains referred to as L1 (1-151) and L2 (299-461) (Ward et al., 2001, supra) separated by a cysteine-rich (CR) region (152-298) consisting of several structural modules with disulfide linkages that align with repeating units present in the TNF receptor and laminin (Ward et al., 1995, Proteins 22:141-53). The crystal structure of the L1-CR-L2 domain has been solved (Garrett et al., 1998, Nature 394:395-99). The L2 domain is followed by three fibronectin type III domains (Marino-Buslje et al., 1998, supra, Mulhern et al., 1998, Trends Biochem Sci 23:465-66, Ward et al., 1999, Growth Factors 16:315-22). The first FnIII domain (FnIII-1, 461-579) is 118 amino acids in length. The second FnIII domain (FnIII-2, 580-798) is disrupted by a major insert sequence (ID) of about 120 amino acids in length. The ID domain includes a furin protease cleavage site that separates the α and β chains of the mature receptor. The third FnIII domain (FnIII-3) is located entirely in the β-chain (799-901) terminating several residues before the transmembrane sequence. The catalytic domain of the IGF-1R tyrosine kinase is located between amino acids positions 973-1229, and its structure has been solved (Favelyukis et al., 2001, Nature Structural Biol 8:1058-63, Pautsch et al., 2001, Structure 9:955-65). The kinase is flanked by two regulatory regions, the juxtamembrane region (930-972) and a 108 amino acid C-terminal tail (1220-1337) (Surmacz et al., 1995, Experimental Cell Res 218:370-80, Hongo et al., 1996, Oncogene 12:1231-38). The two regulatory regions contain tyrosine residues that serve as docking sites for signal transducing proteins when phosphorylated by the activated IGF-1R tyrosine kinase (reviewed by Baserga (ed.), 1998 The IGF-1Receptor in Normal and Abnormal Growth, Hormones and Growth Factors in Development and Neoplasia, Wiley-Liss, Inc., Adams et al., 2000, Cell Mol Life Sci 57:1050-93).

The IGF-1R amino acid sequence is about 70% identical to the insulin receptor (INSR; Swiss-Prot: P06213). The highest homology between the receptors is located in the tyrosine kinase domain (84%); the lowest identity is in the CR region and the C-terminus. The IGF-1R is also highly related (“55% identical) to the insulin related receptor (IRR; Swiss-Prot: P14616).

Human IGF-1R can be activated by the insulin-like growth factors, IGF-1 and IGF-2 and insulin (INS) (Hill et al., 1985, Pediatric Research 19:879-86). IGF-1 and IGF-2 are encoded nonallelic genes (Brissenden et al., 1984, Nature 310: 781-8, Bell et al., 1985, PNAS USA 82: 6450-54), and both genes express alternative proteins related by differential RNA splicing and protein processing. The most common and well-studied mature forms of IGF-1 and IGF-2 are respectively 70 and 67 amino acids in length (Jansen et al., 1983, Nature 306:609-11, Dull et al., 1984, Nature 310: 777-81). These proteins (and their isoforms) are identical at 11/21 positions to the insulin A-peptide, and identical at 12/30 positions with the insulin B-peptide.

IGF-1R is expressed in all cells types in the normal adult animal except for liver hepatocytes and mature B-cells. Human blood plasma contains high concentrations of IGF-1 and IGF-2, and IGF-1 can be detected in most tissues. The receptor is an integral component of the physiological mechanism controlling organ size and homeostasis. Without being bound to a particular theory, the “Somatomedin Hypothesis” states that Growth Hormone (GH) mediated somatic growth that occurs during childhood and adolescence is dependent on the endocrine form of IGF-1 that is mainly produced and secreted by the liver (Daughaday, 2000, Pediatric Nephrology 14: 537-40). The synthesis of hepatic IGF-1 is stimulated by GH release in the pituitary in response to hypothalamic GHRH (GH releasing hormone). The serum concentration of IGF-1 increases over 100 fold between ages 5-15 in humans. The bioavailability of IGF-1 is regulated by IGF binding protein 3 (IGFBP3) with approximately 99% of the growth factor compartmentalized in the bound state. Primary IGF-1 deficiency arising from partial gene deletions, and secondary IGF-1 deficiency resulting from defects in GH production or signaling are not lethal (Woods, 1999, IGF Deficiency in Contemporary Endocrinology The IGF System, R. a. R. Rosenfeld, C. Jr. Totowa, eds, Humana Press, NJ: 651-74). The affected individuals exhibit growth retardation at birth, grow slowly and can face certain CNS abnormalities.

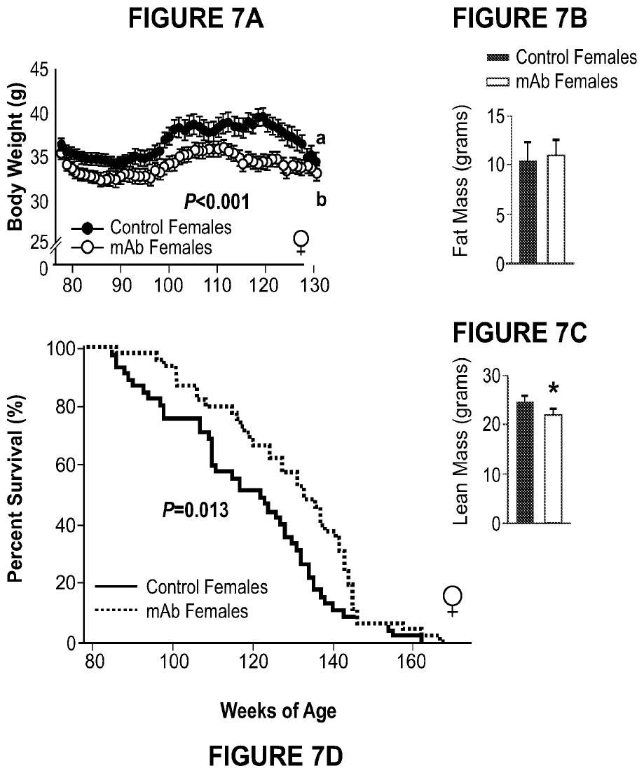

IGF-1R signaling promotes cell growth and survival through the IRS adapter protein-dependent activation of the PI3Kinase/Akt pathway. IGF-1R transmits a signal to its major substrates, IRS-1 through IRS-4 and the Shc proteins (Blakesley et al., 1999, IGF-1 receptor function: transducing the IGF-1 signal into intracellular events in The IGF System, R. G. a. R. Rosenfeld, Jr. C. T. Totowa, ed.s, Humana Press, NJ: 143-63). This results in activation of the Ras/Raf/MAP kinase and PI3 Kinase/Akt signaling pathways. However, induction of Akt-mediated cell survival via IRS is the dominant pathway response upon IGF stimulation of most cells. See FIG. 10 of U.S. Pat. No. 7,871,611.

IGF-1R Inhibitor

In exemplary embodiments, the IGF-1R inhibitor comprises an antigen binding protein that binds to an epitope of IGF-1 or IGF-1R and inhibits IGF-1 binding to IGF-1R. As used herein, the term “IGF-1R inhibitor” is any agent or compound that inhibits or reduces the action of IGF-1R. In some instances, the IGF-1R inhibitor is an IGF-1R antagonist. The terms “IGF-1R inhibitor” and “IGF-1R antagonist” are used interchangeably. Each is a molecule that detectably inhibits at least one function of IGF-1R. Conversely, an “IGF-1R agonist” is a molecule that detectably increases at least one function of IGF-1R. The inhibition caused by an IGF-1R inhibitor need not be complete so long as it is detectable using an assay. Any assay of a function of IGF-1R can be used, examples of which are provided herein and in U.S. Pat. No. 7,871,611, which is incorporated by reference in its entirety. Examples of functions of IGF-1R that can be inhibited by an IGF-1R inhibitor, or increased by an IGF-1R agonist, include binding to IGF-1, IGF-12, and/or another IGF-1R-activating molecule, kinase activity, downstream signaling, and so on. Functions of IGF-1R are known in the art, some of which are described herein. See section entitled “IGF-1R”. Examples of types of IGF-1R inhibitors and IGF-1R agonists include, but are not limited to, IGF-1R binding polypeptides such as antigen binding proteins (e.g., IGF-1R inhibiting antigen binding proteins), antibodies, antibody fragments, and antibody derivatives.

In some aspects, the IGF-1R inhibitor comprises a small molecule that inhibits IGF-1 binding to IGF-1R. Exemplary small molecules that inhibit IGF-1 binding to IGF-1R include, but are not limited to, the compounds set forth in Table 1.

TABLE 1
Small molecule tyrosine kinase inhibitors (TKI) against IGF-1R.

| | | | IC₅₀ (μmol/L) against | | | |
|---|---|---|---|---|---|---|
| Agent | Disclosed in | Class (route) | IGF-1R | IR | Others | Phase |
| Linsitinib | Ji et al., | TKI (oral) ATP- | 0.018 | 0.054 | None | Phase 3 |
| (OSI-906) | AACR Meeting | competitive | | | | |
| | Abstracts. | | | | | |
| | 2007 April;: 2373 | | | | | |
| BMS-754807 | Carboni et al., | TKI (oral) ATP- | <2 | <2 | 11 other kinases | Phase 2 |
| | Mol Cancer Ther. | competitive | nmol/L | nmol/L | <100 nmol/L | |
| | 2009; 8: 3341-3349 | | | | | |
| BVP 51004 | Girnita et al., | Small molecule | 0.038 | No | None | Phase 1 |
| | Cancer Res. | (oral) Not | μmol/L | effect | | |
| | 2004; 64: 236-242 | ATP-competitive | | | | |
| XL 228 | Cortes et al., | TKI (IV) | 1.6 nmol/L | NA | Bcr-Abl: 5 | Phase 1 |
| | American Society | ATP-competitive | (cellular) | | nmol/L | |
| | of Hematology | | | | Bcr-Abl T315I: | |
| | Annual Meeting, | | | | 1.4 nmol/L | |
| | December 2008: | | | | Src: 6.1 | |
| | A3232. | | | | nmol/L | |
| | | | | | Aurora A: 3.1 | |
| | | | | | nmol/L | |
| | | | | | LYN: 2 nmol/L | |
| | | | | | (all cellular) | |
| INSM-18 | Insmed | Phenolic | 31 μmol/L | NA | HER-2: 15 | Phase 1 |
| (NDGA) | | compound | (cellular) | | μmol/L | |
| | | isolated from | | | (cellular | |
| | | creosote bush | | | | |
| | | Larrea | | | | |
| | | divaricate | | | | |
| BMS 536924 | BMS | TKI (oral) ATP- | | | | |
| | | competitive | | | | |

Other exemplary IGF-1R inhibitors include the IGF-1R inhibitors disclosed in U.S. Pat. Nos. 6,804,085; 8,168,410; 7,241,444; 7,914,784; 7,037,498, 7,371,378; 7,378,503; 7,217,796; U.S. Patent Application Publication Nos.: US 2009/0285824, US2004/0086503, US2004/0202651, US2008/0063639, US 2010/0158920, US2004/0202655, US2009/0068110, US2005/0186203, US2005/0136063 and International Publication No. WO 2007/012614, the disclosures of which are incorporated herein by reference in their entireties and for their respective specific teachings of inhibitor compositions and how to make and use them.

As used herein, the term “inhibit” or “reduce” and words stemming therefrom may not be a 100% or complete inhibition or reduction. Rather, there are varying degrees of inhibition or reduction of which one of ordinary skill in the art recognizes as having a potential benefit or therapeutic effect. In this respect, the IGF-1R inhibitor of the present disclosure may inhibit or reduce the action(s) of IGF-1R to any amount or level. In exemplary embodiments, the inhibition provided by the IGF-1R inhibitors is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition). In exemplary embodiments, the reduction provided by the inhibitors of the present disclosure is at least or about a 10% reduction (e.g., at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, at least or about a 95% reduction, at least or about a 98% reduction).

Antigen Binding Proteins

In exemplary aspects, the IGF-1R inhibitor is an antigen-binding protein that binds to IGF-1R, e.g., human IGF-1R. An “antigen binding protein” is a protein comprising a portion that binds to an antigen and, optionally, a scaffold or framework portion that allows the antigen binding portion to adopt a conformation that promotes binding of the antigen binding protein to the antigen. Antigen binding proteins in accordance with the present invention include antigen binding proteins that inhibit a biological activity of IGF-1R. Examples of such biological activities include binding a signaling molecule (e.g., IGF-1 and/or IGF-2), and transducing a signal in response to binding a signaling molecule.

In exemplary aspects, the antigen-binding protein is an antibody or immunoglobulin, or an antigen binding antibody fragment thereof, or an antibody protein product.

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4. In exemplary aspects, the antibody of the present disclosure comprises a non-human constant region. In exemplary aspects, the antibody is a murinized antibody as further described herein.

The antibody can be a monoclonal antibody or a polyclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antibody is a human antibody. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects of the present disclosure, the fusion protein of the present disclosure comprises an antigen-binding fragment of an antibody (a.k.a., antigen-binding antibody fragment, antigen-binding fragment, antigen-binding portion). In exemplary instances, the antigen-binding antibody fragment is a Fab fragment or a F(ab′)₂ fragment. A Fab fragment is a monovalent fragment having the V_{L}, V_{H}, C_{L} and C_{H}1 domains. A F(ab′)₂ fragment is a bivalent fragment having two Fab fragments linked by a disulfide bridge at the hinge region.

The architecture of antibodies has been exploited to create a growing range of alternative formats that span a molecular-weight range of at least about 12-150 kDa and has a valency (n) range from monomeric (n=1), to dimeric (n=2), to trimeric (n=3), to tetrameric (n=4), and potentially higher; such alternative formats are referred to herein as “antibody protein products”. Antibody protein products include those based on the full antibody structure and those that mimic antibody fragments which retain full antigen-binding capacity, e.g., scFvs, Fabs and VHH/VH (discussed below). The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment (fragment, antigen-binding). Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the fusion protein of the present disclosure comprises any one of these antibody protein products. In exemplary aspects, the fusion protein of the present disclosure comprises any one of an scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, and BsAb conjugate.

In exemplary aspects, the antigen binding protein comprises, consists essentially of, or consists of an antibody protein product. In exemplary aspects, the antibody protein product comprises any one of an scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, diabody; a triabody; a tetrabody; a bispecific or trispecific antibody, BsIgG, appended IgG, BsAb fragment, bispecific fusion protein, and BsAb conjugate. In exemplary aspects, the antibody protein product is a Fab′, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83).

A Fd fragment has the V_{H} and C_{H}1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, US App. Pub. No. 05/0202512, 04/0202995, 04/0038291, 04/0009507, 03/0039958, Ward et al., Nature 341:544-546, 1989).

Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. A “CDR grafted antibody” is an antibody comprising one or more CDRs derived from an antibody of a particular species or isotype and the framework of another antibody of the same or different species or isotype.

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

An antigen binding protein may have one or more binding sites. If there is more than one binding site, the binding sites may be identical to one another or may be different. For example, a naturally occurring human immunoglobulin typically has two identical binding sites, while a “bispecific” or “bifunctional” antibody has two different binding sites. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody. In exemplary instances, the antibody protein product is in monomeric form, or polymeric, oligomeric, or multimeric form. A “multi-specific antibody” is an antibody that recognizes more than one epitope on one or more antigens. A subclass of this type of antibody is a “bi-specific antibody” which recognizes two distinct epitopes on the same or different antigens.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes. In exemplary aspects, the antibody is not a human antibody.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293. In exemplary aspects, the antibody is not a humanized antibody.

A murinized antibody has a sequence that differs from the sequence of an antibody derived from a human species by one or more amino acid substitutions, deletions, and/or additions, such that the murinized antibody is less likely to induce an immune response, and/or induces a less severe immune response, as compared to the human species antibody, when it is administered to a murine (e.g., mouse) subject. In one embodiment, certain amino acids in the framework and constant domains of the heavy and/or light chains of the human species antibody are mutated to produce the murinized antibody. In another embodiment, the constant domain(s) from a mouse antibody are fused to the variable domain(s) of a human species. In another embodiment, one or more amino acid residues in one or more CDR sequences of a human antibody are changed to reduce the likely immunogenicity of the human antibody when it is administered to a murine subject, wherein the changed amino acid residues either are not critical for immunospecific binding of the antibody to its antigen, or the changes to the amino acid sequence that are made are conservative changes, such that the binding of the murinized antibody to the antigen is not significantly worse than the binding of the human antibody to the antigen. In exemplary aspects, the antibody is a murinized antibody derived from a human antibody. In exemplary aspects, the antibody is a murinized antibody derived from ganitumab.

In exemplary aspects, the antigen binding protein is a chimeric antibody. The term “chimeric antibody” refers to an antibody that contains one or more regions from one antibody and one or more regions from one or more other antibodies. In one embodiment, one or more of the CDRs are derived from a human anti-IGF-1R antibody. In another embodiment, all of the CDRs are derived from a human anti-IGF-1R antibody. In another embodiment, the CDRs from more than one human anti-IGF-1R antibodies are mixed and matched in a chimeric antibody. For instance, a chimeric antibody may comprise a CDR1 from the light chain of a first human anti-IGF-1R antibody, a CDR2 and a CDR3 from the light chain of a second human anti-IGF-1R antibody, and the CDRs from the heavy chain from a third anti-IGF-1R antibody. Further, the framework regions may be derived from one of the same anti-IGF-1R antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody(-ies) from another species or belonging to another antibody class or subclass. Also included are fragments of such antibodies that exhibit the desired biological activity (i.e., the ability to specifically bind IGF-1R). See, e.g., U.S. Pat. No. 4,816,567 and Morrison, 1985, Science 229:1202-07.

In exemplary aspects, the antigen binding protein is a neutralizing antibody. A “neutralizing antibody” or “an inhibitory antibody” is an antibody that inhibits the binding of IGF-1R to IGF-1 and/or IGF-2 when an excess of the anti-IGF-1R antibody reduces the amount of IGF-1 and/or IGF-2 bound to IGF-1R by at least about 20% using the assay described in Example 9. In various embodiments, the antibody reduces the amount of IGF-1 and/or IGF-2 bound to IGF-1R by at least 30%, 40%, 50%, 60%, 70%, 75%, 80%, 85%, 90%, 95%, 97%, 99%, and 99.9%.

An “activating antibody” is an antibody that activates IGF-1R by at least about 20% when added to a cell, tissue or organism expressing IGF-1R, where “100% activation” is the level of activation achieved under physiological conditions by the same molar amount of IGF-1 and/or IGF-2. In various embodiments, the antibody activates IGF-1R activity by at least 30%, 40%, 50%, 60%, 70%, 80%, 90%, 100%, 125%, 150%, 175%, 200%, 250%, 300%, 350%, 400%, 450%, 500%, 750%, or 1000%. In exemplary aspects, the antigen binding protein is not an activating antibody.

Epitope

In exemplary aspects, the antigen binding protein binds to an epitope within IGF-1R. By “epitope” as used herein is meant the region of or within IGF-1R which is bound by the antigen-binding protein. In some embodiments, the epitope is a linear epitope. By “linear epitope” as used herein refers to the region of or within the IGF-1R which is bound by the binding construct and which region is composed of contiguous amino acids of the amino acid sequence of IGF-1R. The amino acids of a linear epitope are adjacent to each other in the primary structure of IGF-1R. Accordingly, a linear epitope is a fragment or portion of the amino acid sequence of the antigen, i.e., IGF-1R. In other exemplary embodiments, the epitope is a conformational or structural epitope. By “conformational epitope” or “structural epitope” is meant an epitope which is composed of amino acids which are located in close proximity to one another only when the IGF-1R is in its properly folded state. Unlike linear epitopes, the amino acids of a conformational or structural epitope are not adjacent to each other in the primary structure (i.e., amino acid sequence) of the IGF-1R. A conformational or structural epitope is not made of contiguous amino acids of the amino acid sequence of the antigen (IGF-1R).

In exemplary instances, the antigen binding protein binds to an epitope within the extracellular domain of IGF-1R, e.g., human IGF-1R. In exemplary instances, the antigen binding protein binds to an epitope within the L2 domain of the alpha subunit of human IGF-1R, and optionally cross-reacts with an epitope within the L2 domain of the alpha subunit of the mouse IGF-1R. In exemplary aspects, the epitope is within an L2 domain that comprises amino acids 300-460 or amino acids 329-491 of the amino acid sequence of the alpha subunit of IGF-1R. The amino acid sequence of the alpha subunit of human IGF-1R is provided here as SEQ ID NO: 11. In exemplary aspects, the antigen binding protein binds to an epitope within the L2 domain of IGF-1R comprising the sequence of SEQ ID NO: 12. In exemplary aspects, the antigen binding protein binds to an epitope within the L2 domain of IGF-1R comprising the sequence of SEQ ID NO: 13. In exemplary aspects, the antigen binding protein binds to an epitope within the L2 domain of IGF-1R comprising the sequence of SEQ ID NO: 14. In exemplary aspects, the antigen binding protein binds to an epitope within the L2 domain of IGF-1R comprising the sequence of any one of SEQ ID NOs: 12-14 and the antigen binding protein cross-reacts with an epitope within the L2 domain of the alpha subunit of the mouse IGF-1R.

Affinity and Avidity

In exemplary embodiments, the binding strength of the antigen-binding protein to IGF-1R may be described in terms of K_{D}. In exemplary aspects, the K_{D} of the antigen-binding protein provided herein is about 10⁻¹ M, about 10⁻² M, about 10⁻³ M, about 10⁻⁴ M, about 10⁻⁵ M, about 10⁻⁶ M, about 10⁻⁷ M, about 10⁻⁸ M, about 10⁻⁹ M, or less. In exemplary aspects, the K_{D} of the antigen-binding protein provided herein is micromolar, nanomolar, picomolar or femtomolar. In exemplary aspects, the K_{D} of the antigen-binding protein provided herein is within a range of about 10⁻⁴ to 10⁻⁶ M, or 10⁻⁷ to 10⁻⁹ M, or 10⁻¹⁰ to 10⁻¹² M, or 10⁻¹³ to 10⁻¹⁵ M. In exemplary aspects, the antigen-binding protein has high affinity for human IGF-1R, murine IGF-1R, or both. In exemplary aspects, the antibody antigen-binding protein has a K_{D} for human IGF-1R of less than 100 pM, optionally, about 1 pM to about 50 pM. In exemplary aspects, the antigen-binding protein has a K_{D} for human IGF-1R within about 1 pM to about 20 pM or less than about 10 pM. In exemplary aspects, the antibody antigen-binding protein has a K_{D} for murine IGF-1R of less than 100 pM, optionally, about 1 pM to about 75 pM. In exemplary aspects, the antigen-binding protein has a K_{D} for murine IGF-1R within about 1 pM to about 20 pM or less than 10 pM. In exemplary aspects, the antigen-binding protein has a K_{D} for the L2 of human IGF-1R that is less than about 0.5 nM or less than about 0.4 nM. In exemplary aspects, the antigen-binding protein has a K_{D} for the L2 of human IGF-1R that is about 0.05 nM to about 0.5 nM or about 0.05 nM to about 0.4 nM or about 0.05 nM to about 0.35 nM. In exemplary aspects, the antigen-binding protein has a K_{D} for the L2 of human IGF-1R that is about 0.25 nM to about 0.4 nM.

Avidity gives a measure of the overall strength of an antibody-antigen complex. It is dependent on three major parameters: affinity of the antigen-binding protein for the epitope, valency of both the antigen-binding protein and IGF-1R, and structural arrangement of the parts that interact. The greater an antigen-binding protein's valency (number of antigen binding sites), the greater the amount of antigen (IGF-1R) it can bind. In exemplary aspects, the antigen-binding proteins have a strong avidity for IGF-1R. In exemplary aspects, the antigen-binding proteins are multivalent. In exemplary aspects, the antigen-binding proteins are bivalent.

Different antigen binding proteins may bind to different domains or epitopes of IGF-1R or act by different mechanisms of action. Examples include but are not limited to antigen binding proteins that interfere with binding of IGF-1 and/or IGF-2 to IGF-1R or that inhibit signal transduction. The site of action may be, for example, intracellular (e.g., by interfering with an intracellular signaling cascade) or extracellular. An antigen binding protein need not completely inhibit an IGF-1 and/or IGF-2 induced activity to find use in the present invention; rather, antigen binding proteins that reduce a particular activity of IGF-1 and/or IGF-2 are contemplated for use as well.

It has been observed that IGF-1 and IGF-2 each exhibits biphasic binding to IGF-1R. High affinity binding has been reported to have a K_{D} in the range of 0.2 nM; high affinity binding, about ten-fold higher. Thus, in one embodiment, the present disclosure provides an IGF-1R inhibitor that inhibits both the high and low affinity binding of IGF-1 and/or IGF-2 to IGF-1R. In exemplary aspects, the IGF-1R inhibitor inhibits binding of IGF-1 to IGF-1R with an IC50 of about 1 nM to about 10 nM or about 1 to about 5 nM. It has been suggested that the high affinity binding, rather than the low affinity binding, of IGF-1 and/or IGF-2 to IGF-1R is required for the conformation change that activates the tyrosine kinase activity of IGF-1R. Thus, in another embodiment, the IGF-1R inhibitor preferentially inhibits the high affinity binding of IGF-1 and/or IGF-2 to IGF-1R as compared to the low affinity binding.

Structure

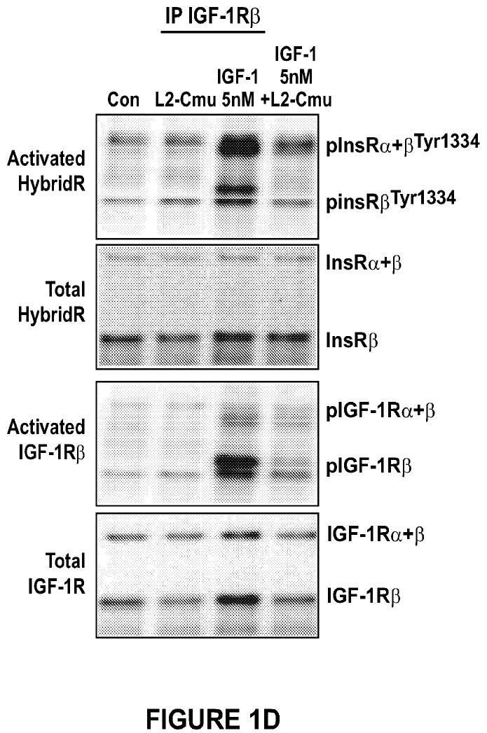

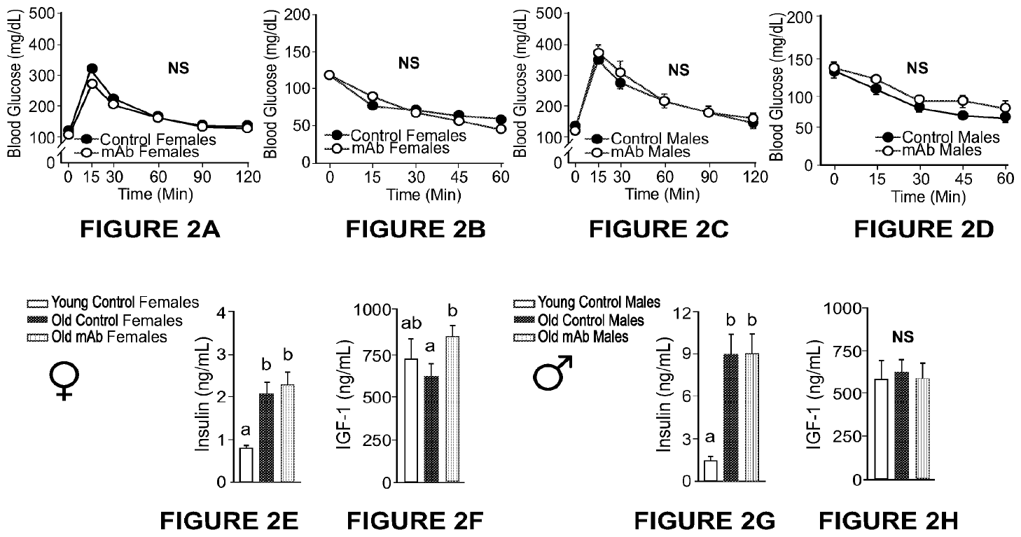

In exemplary instances, the antigen-binding protein comprises a structure (primary structure, amino acid sequence) as described in U.S. Pat. No. 7,871,611, which is incorporated herein by reference. In exemplary aspects, the antigen-binding protein comprises a sequence described in any one of FIGS. 2A, 2B, 3A, and 3B of U.S. Pat. No. 7,871,611. In exemplary aspects, the antigen-binding protein comprises a heavy chain (HC) complementarity-determining region (CDR) 1 amino acid sequence set forth in any one of FIGS. 3A and 3B of U.S. Pat. No. 7,871,611, or a variant sequence thereof which differs by only one or two amino acids or which has at least or about 70% sequence identity; (b) an HC CDR2 amino acid sequence set forth in any one of FIGS. 3A and 3B of U.S. Pat. No. 7,871,611, or a variant sequence thereof which differs by only one or two amino acids or which has at least or about 70% sequence identity; (c) an HC CDR3 amino acid sequence set forth in any one of FIGS. 3A and 3B of U.S. Pat. No. 7,871,611, or a variant sequence thereof which differs by only one or two amino acids or which has at least or about 70% sequence identity; (d) a light chain (LC) CDR1 amino acid sequence set forth in any one of FIGS. 2A and 2B of U.S. Pat. No. 7,871,611, or a variant sequence thereof which differs by only one or two amino acids or which has at least or about 70% sequence identity; (e) an LC CDR2 amino acid sequence set forth in any one of FIGS. 2A and 2B of U.S. Pat. No. 7,871,611, or a variant sequence thereof which differs by only one or two amino acids or which has at least or about 70% sequence identity; (f) an LC CDR3 amino acid sequence set forth in any one of FIGS. 2A and 2B of U.S. Pat. No. 7,871,611, or a variant sequence thereof which differs by only one or two amino acids or which has at least or about 70% sequence identity; or (g) a combination of any two or more of (a)-(f).

In exemplary instances, the antigen-binding proteins comprising (a) a HC CDR1 amino acid sequence set forth in Table A or a sequence selected from the group consisting of: SEQ ID NOs: 121-133, or a variant sequence thereof which differs by only one or two amino acids or which has at least or about 70% sequence identity; (b) an HC CDR2 amino acid sequence set forth in Table A or a sequence selected from the group consisting of: SEQ ID NOs: 134-151, or a variant sequence thereof which differs by only one or two amino acids or which has at least or about 70% sequence identity; (c) an HC CDR3 amino acid sequence set forth in Table A or a sequence selected from the group consisting of: SEQ ID NOs: 152-201, or a variant sequence thereof which differs by only one or two amino acids or which has at least or about 70% sequence identity; (d) a LC CDR1 amino acid sequence set forth in Table A or a sequence selected from the group consisting of: 15, 19, and 55-74, or a variant sequence thereof which differs by only one or two amino acids or which has at least or about 70% sequence identity; (e) an LC CDR2 amino acid sequence set forth in Table A or a sequence selected from the group consisting of: 75-90, or a variant sequence thereof which differs by only one or two amino acids or which has at least or about 70% sequence identity; (f) an LC CDR3 amino acid sequence set forth in Table A or a sequence selected from the group consisting of: 91-120, or a variant sequence thereof which differs by only one or two amino acids or which has at least or about 70% sequence identity; or (g) a combination of any two or more of (a)-(f).

TABLE A

| AB | LC | LC | LC | HC | HC | HC |
|---|---|---|---|---|---|---|
| No. | CDR1 | CDR2 | CDR3 | CDR1 | CDR2 | CDR3 |
| 1 | 55 | 75 | 93 | 121 | 134 | 197 |
| 2 | 15 | 75 | 93 | 121 | 134 | 180 |
| 3 | 15 | 75 | 91 | 121 | 134 | 181 |
| 4 | 15 | 75 | 96 | 124 | 138 | 182 |
| 5 | 15 | 75 | 91 | 121 | 134 | 152 |
| 6 | 15 | 75 | 91 | 121 | 134 | 153 |
| 7 | 15 | 75 | 91 | 121 | 134 | 167 |
| 8 | 15 | 75 | 91 | 121 | 135 | 169 |
| 9 | 19 | 75 | 95 | 121 | 139 | 154 |
| 10 | 15 | 75 | 98 | 121 | 134 | 170 |
| 11 | 56 | 75 | 93 | 121 | 134 | 155 |
| 12 | 63 | 84 | 113 | 128 | 145 | 183 |
| 13 | 15 | 75 | 91 | 121 | 134 | 156 |
| 14 | 15 | 75 | 91 | 121 | 134 | 198 |
| 15 | 15 | 76 | 93 | 121 | 134 | 198 |
| 16 | 15 | 75 | 103 | 121 | 134 | 157 |
| 17 | 15 | 75 | 91 | 121 | 134 | 158 |
| 18 | 67 | 82 | 112 | 128 | 144 | 171 |
| 19 | 15 | 75 | 94 | 121 | 134 | 172 |
| 20 | 15 | 75 | 95 | 121 | 134 | 159 |
| 21 | 57 | 76 | 104 | 123 | 137 | 199 |
| 22 | 74 | 87 | 120 | 127 | 143 | 194 |
| 23 | 15 | 75 | 91 | 121 | 134 | 184 |
| 24 | 15 | 75 | 97 | 133 | 141 | 173 |
| 25 | 15 | 75 | 93 | 121 | 134 | 160 |
| 26 | 58 | 75 | 100 | 121 | 134 | 185 |
| 27 | 68 | 81 | 110 | 132 | 147 | 186 |
| 28 | 73 | 86 | 118 | 126 | 142 | 195 |
| 29 | 15 | 75 | 91 | 121 | 134 | 187 |
| 30 | 15 | 75 | 101 | 121 | 134 | 175 |
| 31 | 71 | 81 | 111 | 130 | 147 | 176 |
| 32 | 15 | 75 | 91 | 121 | 134 | 161 |
| 33 | 15 | 77 | 102 | 121 | 134 | 198 |
| 34 | 15 | 75 | 91 | 121 | 134 | 188 |
| 35 | 64 | 85 | 116 | 130 | 148 | 189 |
| 36 | 63 | 84 | 115 | 124 | 136 | 190 |
| 37 | 15 | 78 | 106 | 121 | 134 | 177 |
| 38 | 59 | 75 | 91 | 121 | 134 | 191 |
| 39 | 15 | 75 | 91 | 121 | 134 | 162 |
| 40 | 69 | 88 | 107 | 129 | 149 | 178 |
| 41 | 72 | 83 | 109 | 131 | 146 | 163 |
| 42 | 15 | 75 | 91 | 122 | 134 | 179 |
| 43 | 70 | 89 | 108 | 129 | 149 | 196 |
| 44 | 15 | 75 | 91 | 121 | 134 | 192 |
| 45 | 15 | 79 | 94 | 122 | 134 | 164 |
| 46 | 15 | 75 | 91 | 121 | 134 | 165 |
| 47 | 60 | 80 | 99 | 121 | 134 | 193 |
| 48 | 15 | 75 | 105 | 131 | 150 | 174 |
| 49 | 65 | 84 | 117 | 124 | 136 | 200 |
| 50 | 66 | 90 | 119 | 125 | 140 | 201 |
| 51 | 62 | 85 | 114 | 129 | 151 | 166 |
| 52 | 61 | 79 | 92 | 121 | 134 | 168 |
| 101 | 306 | 307 | 308 | 309 | 310 | 311 |
| 102 | 306 | 307 | 312 | 313 | 314 | 315 |
| 103 | 316 | 317 | 318 | 319 | 320 | 321 |
| 104 | 322 | 323 | 324 | 325 | 326 | 327 |

In exemplary aspects, the antigen-binding protein comprises a LC CDR1 amino acid sequence, a LC CDR2 amino acid sequence, and a LC CDR3 amino acid sequence set forth in Table A and at least 1 or 2 of the HC CDR amino acid sequences set forth in Table A. In exemplary aspects, the antigen-binding protein comprises a HC CDR1 amino acid sequence, a HC CDR2 amino acid sequence, and a HC CDR3 amino acid sequence set forth in Table A and at least 1 or 2 of the LC CDR amino acid sequences set forth in Table A.

In exemplary embodiments, the antigen-binding protein comprises at least 3, 4, or 5 of the amino acid sequences designated by the SEQ ID NOs: in a single row of Table A. In exemplary embodiments, the antigen-binding protein comprises each of the LC CDR amino acid sequences designated by the SEQ ID NOs: of a single row of Table A and at least 1 or 2 of the HC CDR amino acid sequences designated by the SEQ ID NOs: in of a single row of Table A. In exemplary embodiments, the antigen-binding protein comprises each of the HC CDR amino acid sequences designated by the SEQ ID NOs: of a single row of Table A and at least 1 or 2 of the LC CDR amino acid sequences designated by the SEQ ID NOs: of a single row of Table A. In exemplary embodiments, the antigen-binding protein comprises all 6 of the CDR amino acid sequences designated by the SEQ ID NOs: of a single row of Table A.

In exemplary embodiments, the antigen-binding protein comprises six CDR amino acid sequences of AB1 (SEQ ID NOs: 55, 75, 93, 121, 134, and 197). In exemplary embodiments, the antigen-binding protein comprises six CDR amino acid sequences of AB2 (SEQ ID NOs: 15, 75, 93, 121, 134, and 180). In exemplary embodiments, the antigen-binding protein comprises six CDR amino acid sequences of AB3 (SEQ ID NOs: 15, 75, 91, 121, 134, and 181). In exemplary embodiments, the antigen-binding protein comprises six CDR amino acid sequences of AB4 (SEQ ID NOs: 15, 75, 96, 124, 138, and 182). In exemplary embodiments, the antigen-binding protein comprises six CDR amino acid sequences of AB5 (SEQ ID NOs: 15, 75, 91, 121, 134, and 152). In exemplary embodiments, the antigen-binding protein comprises six CDR amino acid sequences of AB6 (SEQ ID NOs: 15, 75, 91, 121, 134, and 153). In exemplary embodiments, the antigen-binding protein comprises six CDR amino acid sequences of AB7 (SEQ ID NOs: 15, 75, 91, 121, 134, and 167). In exemplary embodiments, the antigen-binding protein comprises six CDR amino acid sequences of AB8 (SEQ ID NOs: 15, 75, 91, 121, 135, and 169). In exemplary embodiments, the antigen-binding protein comprises six CDR amino acid sequences of AB8 (SEQ ID NOs: 15, 75, 91, 121, 135, and 169). In exemplary embodiments, the antigen-binding protein comprises six CDR amino acid sequences of AB9 (SEQ ID NOs: 19, 75, 95, 121, 139, and 154). In exemplary embodiments, the antigen-binding protein comprises six CDR amino acid sequences of AB10 (SEQ ID NOs: 15, 75, 98, 121, 134, and 170). In exemplary embodiments, the antigen-binding protein comprises six CDR amino acid sequences of AB11 (SEQ ID NOs: 56, 75, 93, 121, 134, and 155). In exemplary embodiments, the antigen-binding protein comprises six CDR amino acid sequences of AB12 (SEQ ID NOs: 63, 84, 113, 128, 145, and 183). In exemplary embodiments, the antigen-binding protein comprises six CDR amino acid sequences of AB13 (SEQ ID NOs: 15, 75, 91, 121, 134, and 156). In exemplary embodiments, the antigen-binding protein comprises six CDR amino acid sequences of AB14 (SEQ ID NOs: 15, 75, 91, 121, 134, and 198). In exemplary embodiments, the antigen-binding protein comprises six CDR amino acid sequences of AB15 (SEQ ID NOs: 15, 76, 93, 121, 134, and 198). In exemplary embodiments, the antigen-binding protein comprises six CDR amino acid sequences of AB16 (SEQ ID NOs: 15, 75, 103, 121, 134, and 157). In exemplary embodiments, the antigen-binding protein comprises six CDR amino acid sequences of AB17 (SEQ ID NOs: 15, 75, 91, 121, 134, and 158). In exemplary embodiments, the antigen-binding protein comprises six CDR amino acid sequences of AB18 (SEQ ID NOs: 67, 82, 112, 128, 144, and 171). In exemplary embodiments, the antigen-binding protein comprises six CDR amino acid sequences of AB19 (SEQ ID NOs: 15, 75, 94, 121, 134, and 172). In exemplary embodiments, the antigen-binding protein comprises six CDR amino acid sequences of AB20 (SEQ ID NOs: 15, 75, 95, 121, 134, and 159). In exemplary embodiments, the antigen-binding protein comprises six CDR amino acid sequences of AB21 (SEQ ID NOs: 57, 76, 104, 123, 137, and 199). In exemplary embodiments, the antigen-binding protein comprises six CDR amino acid sequences of AB22 (SEQ ID NOs: 74, 87, 120, 127, 143, and 194). In exemplary embodiments, the antigen-binding protein comprises six CDR amino acid sequences of AB23 (SEQ ID NOs: 15, 75, 91, 121, 134, 184). In exemplary embodiments, the antigen-binding protein comprises six CDR amino acid sequences of AB24 (SEQ ID NOs: 15, 75, 97, 133, 141, and 173). In exemplary embodiments, the antigen-binding protein comprises six CDR amino acid sequences of AB25 (SEQ ID NOs: 15, 75, 93, 121, 134, and 160). In exemplary embodiments, the antigen-binding protein comprises six CDR amino acid sequences of AB8 (SEQ ID NOs: 15, 75, 93, 121, 135, and 169). In exemplary embodiments, the antigen-binding protein comprises six CDR amino acid sequences of AB26 (SEQ ID NOs: 58, 75, 100, 121, 134, and 185). In exemplary embodiments, the antigen-binding protein comprises six CDR amino acid sequences of AB27 (SEQ ID NOs: 68, 81, 110, 132, 147, and 186). In exemplary embodiments, the antigen-binding protein comprises six CDR amino acid sequences of AB28 (SEQ ID NOs: 73, 86, 118, 126, 142, and 195). In exemplary embodiments, the antigen-binding protein comprises six CDR amino acid sequences of AB29 (SEQ ID NOs: 15, 75, 91, 121, 134, and 187). In exemplary embodiments, the antigen-binding protein comprises six CDR amino acid sequences of AB30 (SEQ ID NOs: 15, 75, 101, 121, 134, and 175). In exemplary embodiments, the antigen-binding protein comprises six CDR amino acid sequences of AB31 (SEQ ID NOs: 71, 81, 111, 130, 147, and 176). In exemplary embodiments, the antigen-binding protein comprises six CDR amino acid sequences of AB32 (SEQ ID NOs: 15, 75, 91, 121, 134, and 161). In exemplary embodiments, the antigen-binding protein comprises six CDR amino acid sequences of AB33 (SEQ ID NOs: 15, 77, 102, 121, 134, and 198). In exemplary embodiments, the antigen-binding protein comprises six CDR amino acid sequences of AB34 (SEQ ID NOs: 15, 75, 91, 121, 134, and 188). In exemplary embodiments, the antigen-binding protein comprises six CDR amino acid sequences of AB35 (SEQ ID NOs: 64, 85, 116, 130, 148, and 189). In exemplary embodiments, the antigen-binding protein comprises six CDR amino acid sequences of AB36 (SEQ ID NOs: 63, 84, 115, 124, 136, and 190). In exemplary embodiments, the antigen-binding protein comprises six CDR amino acid sequences of AB37 (SEQ ID NOs: 15, 78, 106, 121, 134, and 177). In exemplary embodiments, the antigen-binding protein comprises six CDR amino acid sequences of AB38 (SEQ ID NOs: 59, 75, 91, 121, 134, and 191). In exemplary embodiments, the antigen-binding protein comprises six CDR amino acid sequences of AB39 (SEQ ID NOs: 15, 75, 91, 121, 134, and 162). In exemplary embodiments, the antigen-binding protein comprises six CDR amino acid sequences of AB40 (SEQ ID NOs: 69, 88, 107, 129, 149, and 178). In exemplary embodiments, the antigen-binding protein comprises six CDR amino acid sequences of AB41 (SEQ ID NOs: 72, 83, 109, 131, 146, and 163). In exemplary embodiments, the antigen-binding protein comprises six CDR amino acid sequences of AB42 (SEQ ID NOs: 15, 75, 91, 122, 134, and 179). In exemplary embodiments, the antigen-binding protein comprises six CDR amino acid sequences of AB43 (SEQ ID NOs: 70, 89, 108, 129, 149, and 196). In exemplary embodiments, the antigen-binding protein comprises six CDR amino acid sequences of AB44 (SEQ ID NOs: 15, 75, 91, 121, 134, and 192). In exemplary embodiments, the antigen-binding protein comprises six CDR amino acid sequences of AB45 (SEQ ID NOs: 15, 79, 94, 122, 134, and 164). In exemplary embodiments, the antigen-binding protein comprises six CDR amino acid sequences of AB46 (SEQ ID NOs: 15, 75, 91, 121, 134, and 165). In exemplary embodiments, the antigen-binding protein comprises six CDR amino acid sequences of AB47 (SEQ ID NOs: 60, 80, 99, 121, 134, and 193). In exemplary embodiments, the antigen-binding protein comprises six CDR amino acid sequences of AB48 (SEQ ID NOs: 15, 75, 105, 131, 150, and 174). In exemplary embodiments, the antigen-binding protein comprises six CDR amino acid sequences of AB49 (SEQ ID NOs: 65, 84, 117, 124, 136, and 200). In exemplary embodiments, the antigen-binding protein comprises six CDR amino acid sequences of AB50 (SEQ ID NOs: 66, 90, 119, 125, 140, and 201). In exemplary embodiments, the antigen-binding protein comprises six CDR amino acid sequences of AB51 (SEQ ID NOs: 62, 85, 114, 129, 151, and 166). In exemplary embodiments, the antigen-binding protein comprises six CDR amino acid sequences of AB52 (SEQ ID NOs: 61, 79, 92, 121, 134, and 168). In exemplary embodiments, the antigen-binding protein comprises six CDR amino acid sequences of AB101 (SEQ ID NOs: 306, 307, 308, 309, 310, and 311). In exemplary embodiments, the antigen-binding protein comprises six CDR amino acid sequences of AB102 (SEQ ID NOs: 306, 307, 312, 313, 314, and 315). In exemplary embodiments, the antigen-binding protein comprises six CDR amino acid sequences of AB103 (SEQ ID NOs: 316, 317, 318, 319, 320, and 321). In exemplary embodiments, the antigen-binding protein comprises six CDR amino acid sequences of AB104 (SEQ ID NOs: 322, 323, 324, 325, 326, and 327).

In exemplary instances, the amino acid sequences of Table A are separated by at least one or more (e.g., at least 2, 3, 4, 5, 6, 7, 8, 9, 10, or more) intervening amino acid(s). In exemplary instances, there are about 10 to about 20 amino acids between the sequences of the LC CDR1 and the LC CDR2 and about 25 to about 40 amino acids between the sequences of the LC CDR2 and the LC CDR3. In exemplary instances, there are about 14 to about 16 amino acids between the sequences of the LC CDR1 and the LC CDR2 and about 30 to about 35 amino acids between the sequences of LC CDR2 and the LC CDR3. In exemplary instances, there are about 10 to about 20 amino acids between the sequences of the HC CDR1 and HC CDR2 and about 25 to about 40 amino acids between the sequences of the HC CDR2 and the HC CDR3. In exemplary instances, there are about 14 to about 16 amino acids between the sequences of the HC CDR1 and HC CDR2 and about 30 to about 35 amino acids between the sequences of the HC CDR2 and HC CDR3.

In exemplary embodiments, the antigen-binding protein comprises (a) a heavy chain variable region amino acid sequence set forth in in Table B or a sequence selected from the group consisting of: 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 301, 303, and 305, or a variant sequence thereof which differs by only one or two amino acids or which has at least or about 70% sequence identity; or (b) a light chain variable region amino acid sequence set forth in Table B or a sequence selected from the group consisting of: 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274 276, 278, 280, 282, 284, 286, 288, 290, 292, 294, 296, 298, 300, 302, and 304, or a variant sequence thereof which differs by only one or two amino acids or which has at least or about 70% sequence identity; or (c) both (a) and (b).

TABLE B

| AB # | LC VAR | HC VAR |
|---|---|---|
| 1 | 202 | 203 |
| 2 | 204 | 205 |
| 3 | 206 | 207 |
| 4 | 208 | 209 |
| 5 | 210 | 211 |
| 6 | 212 | 213 |
| 7 | 214 | 215 |
| 8 | 216 | 217 |
| 9 | 218 | 219 |
| 10 | 220 | 221 |
| 11 | 222 | 223 |
| 12 | 224 | 225 |
| 13 | 226 | 227 |
| 14 | 228 | 229 |
| 15 | 230 | 231 |
| 16 | 232 | 233 |
| 17 | 234 | 235 |
| 18 | 236 | 237 |
| 19 | 238 | 239 |
| 20 | 240 | 241 |
| 21 | 242 | 243 |
| 22 | 244 | 245 |
| 23 | 246 | 247 |
| 24 | 248 | 249 |
| 25 | 250 | 251 |
| 26 | 252 | 253 |
| 27 | 254 | 255 |
| 28 | 256 | 257 |
| 29 | 258 | 259 |
| 30 | 260 | 261 |
| 31 | 262 | 263 |
| 32 | 264 | 265 |
| 33 | 266 | 267 |
| 34 | 268 | 269 |
| 35 | 270 | 271 |
| 36 | 272 | 273 |
| 37 | 274 | 275 |
| 38 | 276 | 277 |
| 39 | 278 | 279 |
| 40 | 280 | 281 |
| 41 | 282 | 283 |
| 42 | 284 | 285 |
| 43 | 286 | 287 |
| 44 | 288 | 289 |
| 45 | 290 | 291 |
| 46 | 292 | 293 |
| 47 | 294 | 295 |
| 48 | 296 | 297 |
| 49 | 298 | 299 |
| 50 | 300 | 301 |
| 51 | 302 | 303 |
| 52 | 304 | 305 |

In exemplary embodiments, the antigen-binding protein comprises a pair of amino acid sequences selected from the group consisting of: (a) SEQ ID NOs: 202 and 203; (b) SEQ ID NOs: 204 and 205; (c) SEQ ID NOs: 206 and 207; (d) SEQ ID NOs: 208 and 209; (e) SEQ ID NOs: 210 and 211; (f) SEQ ID NOs: 212 and 213; (g) SEQ ID NOs: 214 and 215; (h) SEQ ID NOs: 216 and 217; (i) SEQ ID NOs: 218 and 219; (j) SEQ ID NOs: 220 and 221; (k) SEQ ID NOs: 222 and 223; (I) SEQ ID NOs: 224 and 225; (m) SEQ ID NOs: 226 and 227; (n) SEQ ID NOs: 228 and 229; (o) SEQ ID NOs: 230 and 231; (p) SEQ ID NOs: 232 and 233; (q) SEQ ID NOs: 234 and 235; (r) SEQ ID NOs: 236 and 237; (s) SEQ ID NOs: 238 and 239; (t) SEQ ID NOs: 240 and 241; (u) SEQ ID NOs: 242 and 243 (v) SEQ ID NOs: 244 and 245; (w) SEQ ID NOs: 246 and 247; (x) SEQ ID NOs: 248 and 249; (y) SEQ ID NOs: 250 and 251; (z) SEQ ID NOs: 252 and 253; (aa) SEQ ID NOs: 254 and 255; (bb) SEQ ID NOs: 256 and 257; (cc) SEQ ID NOs: 258 and 259; (dd) SEQ ID NOs: 260 and 261; (ee) SEQ ID NOs: 262 and 263; (ff) SEQ ID NOs: 264 and 265; (gg) SEQ ID NOs: 266 and 267; (hh) SEQ ID NOs: 268 and 269; (ii) SEQ ID NOs: 270 and 271; (jj) SEQ ID NOs: 272 and 273; (kk) SEQ ID NOs: 274 and 275; (II) SEQ ID NOs: 276 and 277; (mm) SEQ ID NOs: 278 and 279; (nn) SEQ ID NOs: 280 and 281; (oo) SEQ ID NOs: 282 and 283; (pp) SEQ ID NOs: 284 and 285; (qq) SEQ ID NOs: 286 and 287; (rr) SEQ ID NOs: 288 and 289; (ss) SEQ ID NOs: 290 and 291; (tt) SEQ ID NOs: 292 and 293; (uu) SEQ ID NOs: 294 and 295; (vv) SEQ ID NOs: 296 and 297; (ww) SEQ ID NOs: 298 and 299; (xx) SEQ ID NOs: 300 and 301; (yy) SEQ ID NOs: 302 and 303; and (zz) SEQ ID NOs: 304 and 305.

In exemplary aspects, the antigen-binding protein comprises an amino acid sequence which is similar to an above-referenced amino acid sequence, yet the antigen-binding protein substantially retains its biological function, e.g., its ability to bind to human IGF-1R and inhibit signal transduction through IGF-1R.

In exemplary aspects, the antigen-binding protein comprises an amino acid sequence which differs by only 1, 2, 3, 4, 5, 6, or more amino acids, relative to the above-referenced amino acid sequence(s). In exemplary aspects, the antigen-binding protein comprises a variant sequence of the referenced sequence, which variant sequence differs by only one or two amino acids, relative to the referenced sequence. In exemplary aspects, the antigen-binding protein comprising one or more amino acid substitutions that occur outside of the CDRs, e.g., the one or more amino acid substitutions occur within the framework region(s) of the heavy or light chain. In exemplary aspects, the antigen-binding protein comprising one or more amino acid substitutions yet the antigen-binding protein retains the amino acid sequences of the six CDRs. In exemplary aspects, the antigen-binding protein comprises an amino acid sequence having only 1, 2, 3, 4, 5, 6, or more conservative amino acid substitutions, relative to the above-referenced amino acid sequence(s). As used herein, the term “conservative amino acid substitution” is defined herein as the substitution of one amino acid with another amino acid having similar properties, e.g., size, charge, hydrophobicity, hydrophilicity, and/or aromaticity, and includes exchanges within one of the following five groups:
I. Small aliphatic, nonpolar or slightly polar residues:
   Ala, Ser, Thr, Pro, Gly;
II. Polar, negatively charged residues and their amides and esters:
   Asp, Asn, Glu, Gln, cysteic acid and homocysteic acid;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine
V. Large, aromatic residues:
   Phe, Tyr, Trp, acetyl phenylalanine

In exemplary aspects, the conservative amino acid substitution is an exchange within one of the following groups of amino acids:
I. aliphatic amino acids: Gly, Ala, Val, Leu, Ile
II. non-aromatic amino acids comprising a side chain hydroxyl: Serc Thr
III. amino acids comprising a sulfur side chain: Cys, Met
IV: amino acids comprising a side chain aromatic ring: Phe, Tyr, Trp
V: acidic amino acid: Glu; Asp
VI: basic amino acid: Arg; Lys
VII: amino acid comprising a side chain amide: Gln, Asn
VIII: amino acid comprising a side chain imidazole: His, alpha-dimethyl imidiazole acetic acid (DMIA)
IX: imino acid: Pro, 4-hydroxy-Pro, 4-amino-Pro

In exemplary aspects, the antigen-binding protein comprises an amino acid sequence which has greater than or about 30%, greater than or about 50%, or greater than or about 70% sequence identity to the above-referenced amino acid sequence. In exemplary aspects, the antigen-binding protein comprises an amino acid sequence which has at least 30%, at least 40%, at least 50%, at least 60%, at least 70%, at least 80%, at least 85%, at least 90% or has greater than 90% sequence identity to the above-referenced amino acid sequence. In exemplary aspects, the antigen-binding protein comprises an amino acid sequence that has at least 70%, at least 80%, at least 85%, at least 90% or has greater than 90% sequence identity along the full-length of the above-referenced amino acid sequence.

In exemplary aspects, the antigen-binding protein comprises a variant sequence of the referenced sequence, which variant sequence has at least or about 70% sequence identity, relative to the above-referenced sequence. In exemplary aspects, the antigen-binding protein comprises a variant sequence of the referenced sequence, which variant sequence has at least or about 80% sequence identity, relative to the above-referenced sequence. In exemplary aspects, the antigen-binding protein comprises a variant sequence of the referenced sequence, which variant sequence has at least or about 90% sequence identity, relative to the above-referenced sequence. In exemplary aspects, the antigen-binding protein comprises a variant sequence of the referenced sequence, which variant sequence has at least or about 95% sequence identity, relative to the above-referenced sequence.

In exemplary instances, the antigen-binding protein (e.g., antibody) comprises a light chain (LC) CDR1 comprising the amino acid sequence of SEQ ID NO: 1 or an amino acid sequence comprising at least about 75% (at least about 80%, at least about 85%, at least about 90%, at least about 95% or more) sequence identity to SEQ ID NO: 1; a LC CDR2 comprising the amino acid sequence of SEQ ID NO: 2 or an amino acid sequence comprising at least about 75% (at least about 80%, at least about 85%, at least about 90%, at least about 95% or more) sequence identity to SEQ ID NO: 2; and a LC CDR3 comprising the amino acid sequence of SEQ ID NO: 3 or an amino acid sequence comprising at least about 75% (at least about 80%, at least about 85%, at least about 90%, at least about 95% or more) sequence identity to SEQ ID NO: 3.

In exemplary aspects, the antigen-binding protein (e.g., antibody) comprises a heavy chain (HC) CDR1 comprising the amino acid sequence of SEQ ID NO: 4 or an amino acid sequence comprising at least about 75% (at least about 80%, at least about 85%, at least about 90%, at least about 95% or more) sequence identity to SEQ ID NO: 4; a HC CDR2 comprising the amino acid sequence of SEQ ID NO: 5 or an amino acid sequence comprising at least about 75% (at least about 80%, at least about 85%, at least about 90%, at least about 95% or more) sequence identity to SEQ ID NO: 5; and a HC CDR3 comprising the amino acid sequence of SEQ ID NO: 6 or an amino acid sequence comprising at least about 75% (at least about 80%, at least about 85%, at least about 90%, at least about 95% or more) sequence identity to SEQ ID NO: 6.

In exemplary aspects, the antigen-binding protein (e.g., antibody) comprises a LC that comprises the amino acid sequence of SEQ ID NO: 7, or an amino acid sequence comprising at least about 75% (at least about 80%, at least about 85%, at least about 90%, at least about 95% or more) sequence identity to SEQ ID NO: 7.

In exemplary aspects, the antigen-binding protein (e.g., antibody) comprises a HC that comprises the amino acid sequence of SEQ ID NO: 8, or an amino acid sequence comprising at least about 75% (at least about 80%, at least about 85%, at least about 90%, at least about 95% or more) sequence identity to SEQ ID NO: 8.

In exemplary instances, the antigen-binding protein (e.g., antibody) comprises a light chain (LC) CDR1 comprising the amino acid sequence of SEQ ID NO: 322 or an amino acid sequence comprising at least about 75% (at least about 80%, at least about 85%, at least about 90%, at least about 95% or more) sequence identity to SEQ ID NO: 322; a LC CDR2 comprising the amino acid sequence of SEQ ID NO: 323 or an amino acid sequence comprising at least about 75% (at least about 80%, at least about 85%, at least about 90%, at least about 95% or more) sequence identity to SEQ ID NO: 323; and a LC CDR3 comprising the amino acid sequence of SEQ ID NO: 324 or an amino acid sequence comprising at least about 75% (at least about 80%, at least about 85%, at least about 90%, at least about 95% or more) sequence identity to SEQ ID NO: 324. In exemplary aspects, the antigen-binding protein (e.g., antibody) comprises a heavy chain (HC) CDR1 comprising the amino acid sequence of SEQ ID NO: 325 or an amino acid sequence comprising at least about 75% (at least about 80%, at least about 85%, at least about 90%, at least about 95% or more) sequence identity to SEQ ID NO: 325; a HC CDR2 comprising the amino acid sequence of SEQ ID NO: 326 or an amino acid sequence comprising at least about 75% (at least about 80%, at least about 85%, at least about 90%, at least about 95% or more) sequence identity to SEQ ID NO: 326; and a HC CDR3 comprising the amino acid sequence of SEQ ID NO: 327 or an amino acid sequence comprising at least about 75% (at least about 80%, at least about 85%, at least about 90%, at least about 95% or more) sequence identity to SEQ ID NO: 327.

In exemplary aspects, the antigen-binding protein (e.g., antibody) comprises a LC that comprises the amino acid sequence of SEQ ID NO: 304, or an amino acid sequence comprising at least about 75% (at least about 80%, at least about 85%, at least about 90%, at least about 95% or more) sequence identity to SEQ ID NO: 304.

In exemplary aspects, the antigen-binding protein (e.g., antibody) comprises a HC that comprises the amino acid sequence of SEQ ID NO: 305, or an amino acid sequence comprising at least about 75% (at least about 80%, at least about 85%, at least about 90%, at least about 95% or more) sequence identity to SEQ ID NO: 305.

In exemplary aspects, the antigen-binding protein (e.g., antibody) comprises a heavy chain constant region and a light chain constant region. In exemplary aspects, neither the HC constant region nor the LC constant region are human sequences. In exemplary aspects, the antigen-binding protein (e.g., antibody) comprises a non-human heavy chain constant region and a non-human light chain constant region. In exemplary aspects, the antigen-binding protein (e.g., antibody) comprises a murinized heavy chain constant region and a murinized light chain constant region. In exemplary aspects, the antigen-binding protein (e.g., antibody) comprises a murinized light chain constant region derived from the human light chain constant region sequence of SEQ ID NO: 9. In exemplary aspects, the antigen-binding protein (e.g., antibody) comprises a murinized heavy chain constant region derived from the human heavy chain constant region sequence of SEQ ID NO: 10. In exemplary aspects, the antigen-binding protein (e.g., antibody) comprises a LC constant region that comprises an amino acid sequence comprising at least about 75% (at least about 80%, at least about 85%, at least about 90%, at least about 95% or more) sequence identity to SEQ ID NO: 9 but is not 100% identical to SEQ ID NO: 9. In exemplary aspects, the antigen-binding protein (e.g., antibody) comprises a HC constant region that comprises an amino acid sequence comprising at least about 75% (at least about 80%, at least about 85%, at least about 90%, at least about 95% or more) sequence identity to SEQ ID NO: 10 but is not 100% identical to SEQ ID NO: 10.

Other exemplary IGF-1R antibodies include, but are not limited to the anti-IGF-1R antibodies set forth in Table 2.

TABLE 2
Monoclonal antibodies that target the IGF-1R pathway.

| | | | | Phase 2 |
|---|---|---|---|---|
| Target | Agent name | Disclosed in | Class | Dose |
| IGF-1R | Cixutumumab | Higano et al., | IgG1 | 6 mg/kg qw, |
| | (IMC-A12) | J Clin Oncol. | | 10 mg/kg q2w |
| | | 2009; 27(suppl): | | |
| | | abstr 5142 | | |
| IGF-1R | Figitumumab | Haluska et al., | IgG2 | 20 mg/kg q3w |
| | (CP-751, 871) | Clin Cancer Res. | | |
| | | 2007; 13: | | |
| | | 5834-5840 | | |
| IGF-1R | Dalotuzumab | Atzori et al., | IgG1 | 10 mg/kg q2w |
| | (MK-0646; | Clin Cancer Res. | | |
| | h7C10) | 2011; 17: | | |
| | | 6304-6312 | | |
| IGF-1R | Ganitumab | Tolcher et al., | IgG1 | 18 mg/kg q3w |
| | (AMG749) | J Clin Oncol. | | |
| | | 2009; 27: | | |
| | | 5800-5807 | | |
| IGF-1R | R1507 | Rodon et al., | IgG1 | 9 mg/kg qw |
| | | J Clin Oncol. | | |
| | | 2007; 25(suppl): | | |
| | | abstr 3590 | | |
| IGF-1R | SCH717454 | Seraj et al., | IgG1 | N/A |
| | (19D12) | AACR Meeting | | |
| | | Abstracts. 2009 | | |
| | | April;: 3615 | | |
| IGF-1R | AVE1642 | Tolcher et al., | IgG1 | 8 mg/kg q4w, |
| | (EM164) | J Clin Oncol. | | 12 mg/kg q3w |
| | | 2008; 26(suppl): | | |
| | | abstr 3582 | | |
| IGF-1R | BIIB022 | Dong et al., | IgG4 | N/A |
| | | AACR Meeting | | |
| | | Abstracts. 2008 | | |
| | | April;: 4002 | | |
| EGFR/IGFR | Diabody | Gao et al., | | N/A |
| | (IMC-11F8 to | Cancer Res. | | |
| | EGFR and | 2011; 71: | | |
| | IMC-A12 to | 1029-1040 | | |
| | IGFR.) | | | |

Methods of Making Antibodies

Suitable methods of making antibodies, antigen-binding antibody fragments, and antibody protein products are known in the art. For instance, standard hybridoma methods for producing antibodies are described in, e.g., Harlow and Lane (eds.), Antibodies: A Laboratory Manual, CSH Press (1988), and CA. Janeway et al. (eds.), Immunobiology, 5^{th} Ed., Garland Publishing, New York, N.Y. (2001)). An exemplary method of preparing anti-IGF-1R monoclonal antibodies is provided in U.S. Pat. No. 7,871,611.

Depending on the host species, various adjuvants can be used to increase the immunological response leading to greater antibody production by the host. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Other methods of antibody production are summarized in Table 3.

TABLE 3

| Technique | Exemplary references |
|---|---|
| EBV-hybridoma methods and | Haskard and Archer, J. Immunol. Methods, 74(2), 361-67 |
| Bacteriophage vector expression | (1984), Roder et al., Methods Enzymol., 121, 140-67 |
| systems | (1986), and Huse et al., Science, 246, 1275-81 (1989)). |
| methods of producing antibodies in non- | U.S. Pat. Nos. 5,545,806, 5,569,825, and 5,714,352, and |
| human animals | U.S. Patent Application Publication No. 2002/0197266 |
| inducing in vivo production in the | Orlandi et al (Proc Natl Acad Sci 86: 3833-3837; 1989), |
| lymphocyte population or by screening | and Winter G and Milstein C (Nature 349: 293-299, 1991). |
| recombinant immunoglobulin libraries or | |
| panels of highly specific binding reagents | |
| methods of producing recombinant | Protein production and purification” Nat Methods 5(2): |
| proteins | 135-146 (2008). |
| Phage display | Janeway et al., supra, Huse et al., supra, and U.S. Pat. |
| | No. 6,265,150). Related methods also are described in U.S. |
| | Pat. No. 5,403,484; U.S. Pat. No. 5,571,698; U.S. Pat. |
| | No. 5,837,500; U.S. Pat. No. 5,702,892. The techniques |
| | described in U.S. Pat. No. 5,780,279; U.S. Pat. |
| | No. 5,821,047; U.S. Pat. No. 5,824,520; U.S. Pat. |
| | No. 5,855,885; U.S. Pat. No. 5,858,657; U.S. Pat. |
| | No. 5,871,907; U.S. Pat. No. 5,969,108; U.S. Pat. |
| | No. 6,057,098; and U.S. Pat. No. 6,225,447 |
| Antibodies can be produced by | U.S. Pat. Nos. 5,545,806 and 5,569,825, and Janeway |
| transgenic mice | et al., supra. |

Methods of testing antibodies for the ability to bind to IGF-1 regardless of how the antibodies are produced are known in the art and include any antibody-antigen binding assay, such as, for example, radioimmunoassay (RIA), ELISA, Western blot, immunoprecipitation, SPR, and competitive inhibition assays (see, e.g., Janeway et al., infra, and U.S. Patent Application Publication No. 2002/0197266, and the above section relating to competition assays). Other binding assays, e.g., competitive binding assays or competition assays, which test the ability of an antibody to compete with a second antibody for binding to an antigen, or to an epitope thereof, are known in the art and can be used to test the ability of an antibody to bind to IGF-1. See, e.g., U.S. Patent Application Publication No. US20140178905, Chand et al., Biologicals 46: 168-171 (2017); Liu et al., Anal Biochem 525: 89-91 (2017); and Goolia et al., J Vet Diagn Invest 29(2): 250-253 (2017). Also, other methods of comparing two antibodies are known in the art, and include, for example, surface plasmon resonance (SPR). SPR can be used to determine the binding constants of the antibody and second antibody and the two binding constants can be compared.

Pharmaceutical Compositions

Compositions comprising an IGF-1R inhibitor of the present disclosure are provided herein. The compositions in some aspects comprise an IGF-1R inhibitor of the present disclosure in isolated and/or purified form. In some aspects, the composition comprises a single type (e.g., structure) of an IGF-1R inhibitor of the present disclosure, or comprises a combination of two or more different types (e.g., different structures) of IGF-1R inhibitors described herein.

In exemplary aspects, the composition comprises agents which enhance the chemico-physico features of the IGF-1R inhibitor, e.g., via stabilizing, for example, the IGF-1R inhibitor at certain temperatures (e.g., room temperature), increasing shelf life, reducing degradation, e.g., oxidation protease mediated degradation, increasing half-life of, for example, the IGF-1R inhibitor, etc.

In exemplary aspects of the present disclosure, the composition additionally comprises a pharmaceutically acceptable carrier, diluents, or excipient. In some embodiments, the IGF-1R inhibitor as presently disclosed (hereinafter referred to as “active agents”) is formulated into a pharmaceutical composition comprising the active agent, along with a pharmaceutically acceptable carrier, diluent, or excipient. In this regard, the present disclosure further provides pharmaceutical compositions comprising an active agent (i.e., any of the IGF-1R inhibitors of the present disclosure), which pharmaceutical composition is intended for administration to a subject, e.g., a mammal.

In some embodiments, the active agent is present in the pharmaceutical composition at a purity level suitable for administration to a patient. In some embodiments, the active agent has a purity level of at least about 90%, about 91%, about 92%, about 93%, about 94%, about 95%, about 96%, about 97%, about 98% or about 99%, and a pharmaceutically acceptable diluent, carrier or excipient. In some embodiments, the compositions contain an active agent at a concentration of about 0.001 to about 30.0 mg/ml.

In exemplary aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In exemplary aspects, the pharmaceutical composition comprises formulation materials that are nontoxic to recipients at the dosages and concentrations employed. In specific embodiments, pharmaceutical compositions comprising an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as bcnzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbatc, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18” Edition, (A. R. Genrmo, ed.), 1990, Mack Publishing Company.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In exemplary embodiments, the pH of the pharmaceutical composition is between 5.5 and 7.5.

Routes of Administration

With regard to the present disclosure, the active agent, or pharmaceutical composition comprising the same, can be administered to the subject via any suitable route of administration. For example, the active agent can be administered to a subject via parenteral, nasal, oral, pulmonary, topical, vaginal, or rectal administration. The following discussion on routes of administration is merely provided to illustrate exemplary embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The active agent of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-I53-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-3-aminopropionates, and 2-alkyl-imidazoline quaternary ammonium salts, and (e) mixtures thereof.

The parenteral formulations in some embodiments contain from about 0.5% to about 25% by weight of the active agent of the present disclosure in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Dose and Dosing

Some aspects of the invention involve dose and or dosing schedules, including duration of administration specifically selected for healthspan indications. It will be appreciated that methods targeted to healthspan indications may involve different dosing parameters than methods of using the same agents for acute therapy indications, such as cancer therapy.

In some variations, the dose for the healthspan indication is lower than the dose for a cancer indication, e.g., because different acceptable tolerances for side-effect and/or different levels of targeted inhibition. Similarly, in some variations, the dose period for a healthspan indication is longer term than for a cancer indication, and may involve more administrations, optionally with periods of drug holiday.

In some variations, the dose contemplated is expressed numerically as a function of the subject/patient's mass or estimated surface area. For example, in some variations, the IGF-1R antigen binding protein is administered to the subject at a dose less than or equal to about 12 mg of IGF-1R antigen binding protein per kg of the subject's body weight; or less than or equal to about 10 mg/kg of body weight. For example, in some variations, the dose is about 3-6 mg/kg of body weight.

In some variations, the dose for administration can be expressed in terms of avoidance of undesirable side effects, which can be measured in vivo in dose-response studies, or evaluated in samples isolated from a subject, or estimated from animal or in vitro assays. For instance, in some variations of the invention, the IGF-1R inhibitor is administered at a dose that is no more than 75% of maximum tolerated dose, or no more than 65% of maximum tolerated dose, or no more than 50% of maximum tolerated dose, or no more than 40% of maximum tolerated dose, or no more than 35% of maximum tolerated dose, or no more than 25% of maximum tolerated dose. For example, in some instances, the IGF-1R inhibitor is administered at a dose that is 25-50% of maximum tolerated dose.

In some variations, the dose for administration can be expressed in terms of desired biological effect, which can be measured in vivo, or in samples isolated from a subject, or estimated from animal or in vitro assay.

In some variations, the method or use of the invention comprises repeat administering of the IGF-1R inhibitor to the subject. In some variations, the repeat administering of the IGF-1R inhibitor is performed over a period of at least 2, 3, 4, 5, or 6 months.

For example, the repeated administering of the IGF-1R inhibitor is performed over a period of at least 1, 2, 3, 4, 5, 6, 7, 8, 9, 10, 15, or 20 years. In some variations, the dosing is repeated over as long a period as a beneficial effect on the healthspan parameter is achieved or achievable.

In some variations, the IGF-1R inhibitor is administered for a first period of time, and has a durable effect, and the method includes a drug holiday period during which no IGF-1R inhibitor is administered. For example, in some variations, the method includes periods of administering separated by periods of drug holiday of at least 2 months, for example, periods of 2-24 months; or periods of treatment separated by periods of drug holiday of 3-12 months; or periods of treatment separated by periods of drug holiday of 3-6 months. In exemplary aspects, the method comprises about 1-month to about 2-month periods of administering the IGF-1R inhibitor, separated by periods of drug holiday of at least 3 or 4 months. In exemplary aspects, the method comprises at least one cycle or two cycles of a period of about X weeks of administering the IGF-1R inhibitor followed by a period of about Y weeks of a drug holiday, wherein X is 4, 5, 6, 7, 8, 9, or 10 and Y is 8, 9, 10, 11, 12, 13, 14, 15, or 16. In related aspects, X and/or Y are defined as ranges using these values, e.g, 4-10 or 5-9 for X and 8-16 or 9-15 for Y, with all subranges contemplated. In exemplary aspects, the method comprises at least one cycle or at least 2 cycles of a period of about X weeks of administering the IGF-1R inhibitor followed by a period of about 2X weeks of a drug holiday. In some aspects, the method comprises at least 1 cycle or at least 2 cycles comprising a 1-month period of administering the IGF-1R inhibitor followed by a 3-month period of drug holiday. In some aspects, the method comprises at least 1 cycle or at least 2 cycles comprising a 2-month period of administering the IGF-1R inhibitor followed by a 4 month period of drug holiday. In some aspects, the method comprises at least 2, 3, 4, 5, or 6 cycles comprising a 4-month period of administering the IGF-1R inhibitor followed by an 8-month period of drug holiday. In exemplary aspects, during the periods of administering the IGF-1R inhibitor, the IGF-1R inhibitor is administered to the patient once a day, once a week, once every 2 weeks, once every 3 weeks, or once every 4 weeks. In exemplary instances, during the periods of administering the IGF-1R inhibitor, the IGF-1R inhibitor is administered to the patient once every two weeks.

In some variations, one or more biomarkers are used to guide a period of IGF-1R inhibitor administration and/or guide a period of drug holiday.

For example, in some variations, the composition comprising the IGF-1R inhibitor is administered in an amount and for a duration effective to alter transcription in muscle or adipose tissue, and wherein the period of the drug holiday is commensurate with the period of altered transcription. For example, in some variations, the composition comprising the IGF-1R inhibitor is administered in an amount and for a duration effective to reduce transcription in muscle or adipose tissue, and wherein the period of the drug holiday is commensurate with the period of reduced transcription. In exemplary aspects, the transcription is the transcription of genes involved in inflammation or oxidative stress.

In some variations, serum IGF-1 is used as a biomarker for modulating dose and dosing, where the IGF-1R inhibitor is administered in an amount and at a frequency to cause a measurable increase in serum IGF-1 in a subject, compared to a pre-administration baseline measurement. For example, a dose or dosing of IGF-1R inhibitor is selected to cause an increase of at least 5%, 10%, 15%, 20%, or 25% compared to the baseline. In some variations, the target increase is 5%-50% or any integer subrange thereof (e.g., 5%-45%, 10%-35%, and so on). In some variations, serum IGF-1 is measured periodically, and dose or dosing schedule of the IGF-1R inhibitor is adjusted to maintain the elevated IGF-1. In some variations, the IGF-1 is measured by immunoassay.

In some variations, the IGF-1R inhibitor is administered to the subject for 1 or 2 treatment periods per year, wherein each of the treatment periods is less than about 6 months. For instance, in some variations, the treatment periods is about 1 month to about 4 months. In some variations, the IGF-1R inhibitor is administered to the subject about every 2 weeks during the treatment period.

All routes of administration are contemplated. In some variations, the IGF-1R inhibitor is an IGF-1R antigen binding protein that is administered by subcutaneous injection.

In some variations, intravenous injection is contemplated. In some variations where an effect is desired in a particular tissue, such as cardiac tissue, then site-specific injection is contemplated. In variations of the invention for achieving a neurological effect, direct injection to the brain or cerebrospinal fluid is contemplated.

In some variations, it may be possible to titrate down the effective or maintenance dose for a subject, or it may be necessary to titrate up the dose as a subject ages. Dose adjustment is contemplated as an aspect of the methods and uses herein. For example, in some variations of the methods or uses of the invention, the method or use further comprises reducing dose of the IGF-1R inhibitor for subsequent administrations to eliminate adverse side effects, and administering the reduced dose. In some variations, the IGF-1R is administered at a dose that does not elevate blood glucose more than 10%.

Combination Therapies

Additional variations of the invention include combination therapies with one or more agents to have an additive or more than additive (e.g., a synergistic) beneficial effect on a healthspan parameter.

In some variations, the method or use of the invention further comprises administering to the subject an m-Tor inhibitor such as rapamycin and other rapalogues. Other mTor inhibitors include sirolimus, temsirolimus, everolimus, ridaforolimus, and mTor kinase inhibitors.

In some variations, the method or use of the invention further comprises administering metformin to the subject. In some variations, the method or use of the invention further comprises administering acarbose to the subject.

In some variations, the method or use of the invention further comprises administering to the subject an agent which inhibits BCL-2, BCL-XL, PI3K, or Mdm-2. In some variations, the method or use of the invention further comprises administering to the subject one or more of: venetoclax, navitoclax, ABT-737, AMG511, AMG232, BM-1197, NVP-BKM120, NVP-BEZ235, RG7112, R05503781, SAR405838, DS-3032b, CGM-097, HDM201, MK4828, RG7388ALRN-6924, and combinations thereof.

In exemplary aspects, especially when the subject is female, the method further comprises administering a hormone replacement drug, rapamycin, or rapalogue. In some variations, especially wherein the subject is female, the method further comprises administering an estrogen replacement therapy to the subject.

In exemplary aspects, especially when the subject is female, the method further comprises administering metformin, acarbose, rapamycin, or an anti-inflammatory medication. In some variations, especially wherein the subject is male, the method further comprises administering acarbose to the subject.

For combination therapies, simultaneous and sequential administration, in any order, are contemplated.

The following examples are given merely to illustrate the present disclosure and not in any way to limit its scope.

### EXAMPLES

### Example 1

This example demonstrates that late-life targeting of the IGF-1 receptor improves healthspan and lifespan or survival in female mice.

Summary

Diminished growth factor signaling can improve longevity in laboratory models, while a similar reduction in the somatotropic axis is favorably linked to human aging and exceptional longevity. Given the conserved role of this pathway on lifespan or survival, therapeutic strategies, such as insulin-like growth factor-1 receptor (IGF-1R) monoclonal antibodies (mAbs), represent a promising translational tool to target human aging. To this end, we performed a preclinical study in 18 mo old male and female mice treated with vehicle or an IGF-1R mAb (L2-Cmu, Amgen Inc), and determined effects on aging outcomes. L2-Cmu preferentially improved female healthspan and lifespan or survival (P=0.029), along with a reduction in neoplasms and systemic inflammation (P≤0.05). Thus, consistent with other models, pharmacologic targeting of IGF-1R signaling is most beneficial to female health and survival. Importantly, these effects could be achieved at advanced ages, providing pre-clinical evidence that IGF-1R mAbs or other IGF-1R inhibitors have efficacy as agents to improve healthspan or counteract deleterious effects of aging and serving as a potential

Results

L2-Cmu is a Selective Antagonist to the Murine IGF-1R and InsR/IGF-1R Hybrids.

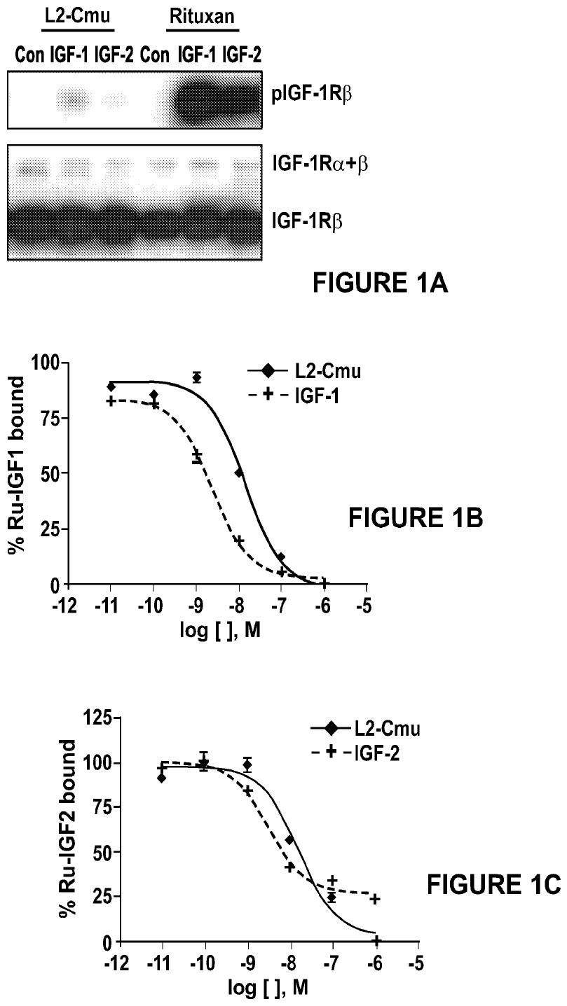

L2-Cmu was developed as a “murinized” version of the antibody L2-C monoclonal antibody at Amgen Inc. (Thousand Oaks, Calif.)²¹. Western blotting and Biacore analysis confirmed that L2-Cmu binds to and inhibits IGF-IR activation by IGF-1 (Ki=3.3 nM) and IGF-2 (Ki=3.3 nM) (FIG. 1a; Table E1), which was verified in the IGEN format (FIG. 1b-c). Selective inhibition of IGF-1R and InsR/IGF-1R hybrid receptors was further confirmed in NIH-3T3 mouse fibroblasts cells (FIG. 1d).

TABLE E1
Biacore measurement of L2-Cmu monoclonal antibody binding

| | Kd | IGF-1 Ki | IGF-1 | IGF-2 Ki | IGF-2 |
|---|---|---|---|---|---|
| mAb | (nM) | (nM) | Max (%) | (nM) | Max (%) |
| L2-Cmu | 0.30 | 3.3 | 99 | 3.3 | 99 |
| αIR3 | 0.33 | >1000 | 31 | >1000 | NI |

Biacore measurement of antibody binding to murine IGF-1R(ECD)-C3-mFc was measured in parallel by the kinetic method. A precise value was not obtained due to limitations in the amount of mu IGF-1R(ECD)-muFc. Ligand blocking measured in the IGEN format with murine IGF-1R(ECD)-C3-mFc and human Ru labeled IGF-1 and IGF-2. NI=No inhibition.

Chronic L2-Cmu Treatment is Well Tolerated in Older Mice

We next performed a 6 mo feasibility study with weekly L2-Cmu intraperitoneal (i.p.) injections (20 mg/kg) in 18 mo old male and female mice, to carefully characterize the safety and efficacy of chronic IGF-1R modulation in aging animals. In females, L2-Cmu mAb treatment led to a slight, albeit significant reduction in red blood cells (RBCs), hemoglobin (Hb), hematocrit (Hct), white blood cells (WBCs), lymphocytes (Table E2) and serum phosphorus (Table E3), though most values, with the exception of WBCs, remained within the ‘normal range’, and were less severe than previously reported with ganitumab in young CD1 mice22. In males, no significant effects of L2-Cmu were observed on WBCs (Table E4) or RBCs (Table E4), but total protein, globulin and ALT were slightly increased, while serum creatinine was reduced (Table E5).

TABLE E2
Female red and white blood cell counts following 6 mo mAb Treatment.

| | Con | mAb | | |
|---|---|---|---|---|
| | Females | Females | p- | Reference |
| Parameter | (n = 5-7) | (n = 8) | value | Range |
| RBC 10\hat{ }3/uL | 9.8 ± 0.3 | 8.1 ± 0.2 | 0.005 | 5.5-10.5 |
| Hemoglobin g/dL | 15.7 ± 0.3 | 13.8 ± 0.3 | 0.008 | 13.0-15.0 |
| Hematocrit % | 49.8 ± 1.7 | 42.4 ± 1.9 | 0.048 | 33-50 |
| MCV Mean Corpuscular | 50.3 ± 0.4 | 52.1 ± 1.5 | 0.39 | |
| Volume fL | | | | |
| MCH mean corpuscular | 16.0 ± 0.5 | 17.0 ± 0.3 | 0.13 | |
| hemoglobin pg | | | | |
| MCHC mean corpuscular | 31.9 ± 1.1 | 32.8 ± 1.4 | 0.66 | |
| hemoglobin concentration | | | | |
| g/dL | | | | |
| Platelet Count 10\hat{ }3/uL | 849.3 ± 56.7 | 901.6 ± 139.2 | 0.82 | |
| WBC | 6.0 ± 0.4‡ | 3.6 ± 0.7 | 0.02 | 5.5-10.5 |
| Neutrophils/uL | 1081 ± 69.7‡ | 635.8 ± 106.4 | 0.06 | |
| Neutrophils % | 19.8 ± 7.9%‡ | 20.4 ± 4.3 | 0.21 | |
| Bands | 0 | 0 | | |
| Lymphocytes/uL | 4166 ± 358‡ | 2781 ± 610 | 0.04 | |
| Lymphocytes % | 75 ± 2 | 73 ± 6 | 0.82 | |
| Monocytes/uL | 464 ± 248 | 123 ± 24 | 0.17 | |
| Monocytes % | 3.6 ± 0.8 | 4.7 ± 1.6 | 0.56 | |
| Eosinophils/uL | 79.4 ± 32.7 | 52.9 ± 29.7 | 0.55 | |
| Eosinophils % | 1.6 ± 0.7 | 2.2 ± 1.3 | 0.68 | |
| Basophils/uL | 0 | 8.1 ± 7.6 | 0.34 | |
| Basophils % | 0 | 0.13 ± 0.13 | 0.37 | |

Data are means ± SE.
‡Two statistical outliers were removed from this group.

TABLE E3
Female blood chemistries following 6 mo mAb Treatment.

| | Con | mAb | | |
|---|---|---|---|---|
| | Females | Females | p- | Reference |
| Parameter | (n = 7) | (n = 8) | value | Range |
| Total Protein | 5.2 ± 0.1 | 5.3 ± 0.1 | 0.13 | 4.5-6.5 |
| g/dL | | | | |
| Albumin g/dL | 2.8 ± 0.1 | 2.8 ± 0.1 | 0.84 | 2.4-4.4 |
| Globulin g/dL | 2.4 ± 0.1 | 2.6 ± 0.1 | 0.12 | 2.4-4.4 |
| AST U/L | 163.4 ± 23.1 | 142.0 ± 18.2 | 0.46 | 10-45 |
| ALT U/L | 48.6 ± 10.9 | 31.0 ± 3.4 | 0.13 | 10-35 |
| Alk Phosphatase | | | | |
| U/L | 86.1 ± 8.4 | 101.5 ± 16.9 | 0.43 | 15-45 |
| Total Bilirubin | | | | |
| mg/dL | 0.17 ± 0.18 | 0.20 ± 0.04 | 0.34 | 0-1 |
| Urea Nitrogen | | | | |
| mg/dL | 17.0 ± 0.9 | 15.8 ± 1.2 | 0.44 | 9-30 |
| Creatinine mg/dL | 0.19 ± 0.01 | 0.20 ± 0.01 | 0.30 | 0.4-1.0 |
| Phosphorus | 7.5 ± 0.3 | 6.4 ± 0.3 | 0.02 | 4.2-8.5 |
| mg/dL | | | | |
| Glucose mg/dL | 150.6 ± 10.5 | 160.0 ± 6.2 | 0.48 | 60-125 |
| Calcium mg/dL | 9.2 ± 0.2 | 8.2 ± 0.6 | 0.16 | 8-12 |
| Sodium mEq/L | 148.7 ± 0.9 | 146.9 ± 0.6 | 0.10 | 140-160 |
| Potassium mEq/L | 5.6 ± 0.3 | 5.6 ± 0.4 | 0.97 | 4.3-5.8 |
| Na/K Ratio | 26.7 ± 1.2 | 26.8 ± 1.6 | 0.99 | |
| Chloride mEq/L | 112.1 ± 1.2 | 111.8 ± 0.6 | 0.76 | 90-110 |
| Cholesterol | 109.6 ± 6.0 | 106.9 ± 4.2 | 0.71 | 50-250 |
| jMg/dL | | | | |
| CPK U/L | 680.3 ± 243.8 | 890.9 ± 178.4 | 0.48 | |

Data are means ± SE.

TABLE E4
Male red and white blood cell counts followina 6 mo mAb Treatment.

| | Con | mAb | | |
|---|---|---|---|---|
| | Males | Males | p- | Reference |
| Parameter | (n = 7) | (n = 8) | value | Range |
| RBC 10\hat{ }3/uL | 8.0 ± 0.8 | 7.67 ± 0.2 | 0.79 | 5.5-10.5 |
| Hemoglobin g/dL | 13.1 ± 1.3 | 12.89 ± 0.3 | 0.93 | 13.0-15.0 |
| Hematocrit % | 41.0 ± 4.3 | 40.14 ± 1.9 | 0.90 | 33-50 |
| MCV Mean Corpuscular | 50.9 ± 0.9 | 51.43 ± 1.5 | 0.67 | |
| Volume fL | | | | |
| MCH mean corpuscular | 16.3 ± 0.5 | 17.15 ± 0.3 | 0.25 | |
| hemoglobin pg | | | | |
| MCHC mean corpuscular | 32.0 ± 1.0 | 33.4 ± 1.4 | 0.34 | |
| hemoglobin concentration | | | | |
| g/dL | | | | |
| Platelet Count 10\hat{ }3/uL | 1040.7 ± 182.9 | 1011 ± 166.8 | 0.91 | |
| WBC | 5.1 ± 0.8‡ | 5.3 ± 1.0 | 0.91 | 5.5-10.5 |
| Neutrophils/uL | 1207 ± 303‡ | 1340 ± 357 | 0.79 | |
| Neutrophils % | 21.8 ± 3.2‡ | 28.6 ± 5.3 | 0.41 | |
| Bands | 0 | 0 | | |
| Lymphocytes/uL | 3676 ± 484‡ | 3726 ± 837 | 0.96 | |
| Lymphocytes % | 73.5 ± 6.2‡ | 66.8 ± 5.2 | 0.42 | |
| Monocytes/uL | 164.7 ± 55.5 | 122.5 ± 45.1 | 0.55 | |
| Monocytes % | 2.1 ± 0.3 | 2.3 ± 0.5 | 0.86 | |
| Eosinophils/uL | 183.1 ± 76.7 | 93.0 ± 42.2 | 0.30 | |
| Eosinophils % | 2.4 ± 1.0 | 3.2 ± 1.2 | 0.65 | |
| Basophils/uL | 0 | 0 | | |
| Basophils % | 0 | 0 | | |

Data are means ± SE.
‡One statistical outlier was removed from this group.

TABLE E5
Male blood chemistries following 6 mo mAb Treatment.

| | Con | mAb | | |
|---|---|---|---|---|
| | Males | Males | p- | Reference |
| Parameter | (n = 7) | (n = 8) | value | Range |
| Total Protein | 4.7 ± 0.2 | 5.8 ± 0.1 | 0.002 | 4.5-6.5 |
| g/dL | | | | |
| Albumin g/dL | 2.4 ± 0.1 | 2.6 ± 0.1 | 0.34 | 2.4-4.4 |
| Globulin g/dL | 2.3 ± 0.1 | 3.1 ± 0.1 | <0.001 | 2.4-4.4 |
| AST U/L | 108.3 ± 21.5 | 83.2 ± 8.4 | 0.34 | 10-45 |
| ALT U/L | 20.7 ± 3.6 | 33.9 ± 3.4 | 0.02 | 10-35 |
| Alk Phosphatase | | | | |
| U/L | 42.6 ± 5.5 | 47.7 ± 5.4 | 0.53 | 15-45 |
| Total Bilirubin | | | | |
| mg/dL | 0.17 ± 0.03 | 0.11 ± 0.01 | 0.10 | 0-1 |
| Urea Nitrogen | | | | |
| mg/dL | 20.3 ± 1.4 | 18.3 ± 1.2 | 0.31 | 9-30 |
| Creatinine mg/dL | 0.21 ± 0.01 | 0.16 ± 0.02 | 0.04 | 0.4-1.0 |
| Phosphorus | 7.3 ± 0.5 | 7.5 ± 0.3 | 0.74 | 4.2-8.5 |
| mg/dL | | | | |
| Glucose mg/dL | 154.1 ± 21.5 | 136.4 ± 6.9 | 0.45 | 60-125 |
| Calcium mg/dL | 9.3 ± 0.1 | 9.1 ± 0.2 | 0.47 | 8-12 |
| Sodium mEq/L | 152.3 ± 0.7 | 150.7 ± 0.8 | 0.19 | 140-160 |
| Potassium mEq/L | 5.6 ± 0.3 | 5.9 ± 0.1 | 0.47 | 4.3-5.8 |
| Na/K Ratio | 27.6 ± 1.5 | 25.7 ± 0.8 | 0.31 | |
| Chloride mEq/L | 112.0 ± 1.1 | 109.8 ± 0.5 | 0.11 | 90-110 |
| Cholesterol | 123.6 ± 15.3 | 139.7 ± 19.5 | 0.56 | 50-250 |
| Mg/dL | | | | |
| CPK U/L | 827.2 ± 244.0 | 652.5 ± 95.6 | 0.55 | |

Data are means ± SE.

Chronic L2-Cmu Treatment Leads to Sex-Specific Metabolic Effects without Perturbing Glucose Homeostasis in Older Mice

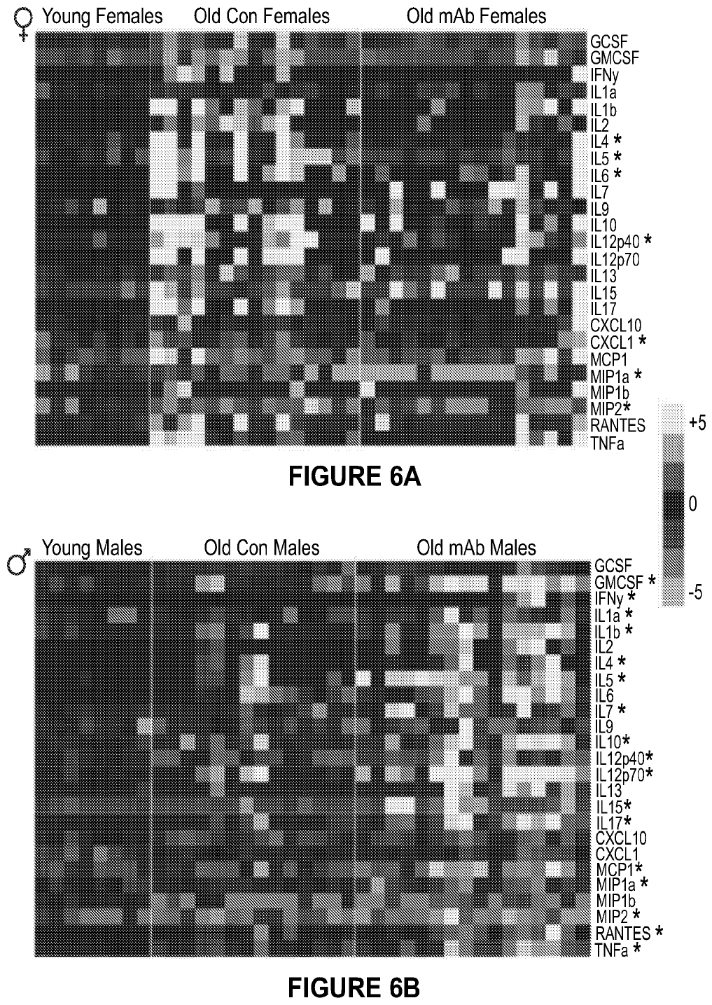

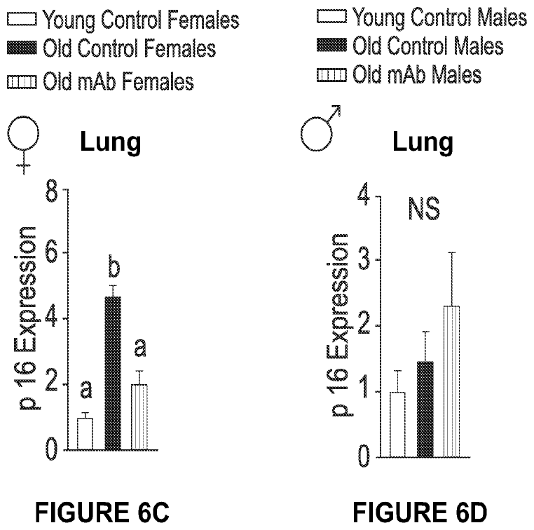

We next carefully evaluated effects of chronic IGF-1R mAb treatment on energy and glucose homeostasis in an initial cohort of older mice. In females, no significant effect on body weight, composition or energy balance was observed following 6 mo L2-Cmu treatment (FIG. 8a-g). In contrast, body weight was numerically decreased while lean mass was significantly reduced in mAb-treated males (FIG. 8g-h; P≤0.05), without effects on adiposity or energy balance (FIG. 8i-k), though an increase in the respiratory exchange ratio (RER), indicative of increased carbohydrate utilization, was detected in mAb-treated males (FIG. 8l). However, mAb treatment did not perturb glucose homeostasis (FIG. 2a-d) or insulin levels (FIG. 2e,g) in males or females. L2-Cmu led to a modest numerical increase in circulating IGF-1 levels in older females (FIG. 2f; Main effect P=0.066), consistent with inhibition of pituitary feedback in these mice²², while IGF-1 levels in males were unaffected by treatment or age in this strain (FIG. 2h). Interestingly, mAb treatment prevented the age-related rise in hypothalamic IGF-1R levels in both sexes, while resulting in reduced cortical IGF-1R levels in males (P≤0.05), without significant effects on lung IGF-1R levels (FIG. 2i-j). Meanwhile, InsR levels were unaffected by mAb treatment in both sexes (FIG. 2k-l). Alterations in downstream components of the Insulin/IGF-1 signaling pathway were observed with mAb treatment, including increased total Akt levels in female lung (FIG. 9a; P≤0.05), and attenuated S6 activation in male hypothalamus (FIG. 10c; P≤0.05).

Therapeutic Modulation of IGF-1 Action Preferentially Benefits Female Healthspan

We next evaluated effects on functional healthspan domains related to neuromuscular and physical performance following 5-6 mo of mAb treatment. With aging, females (FIG. 3a-c) and males (FIG. 3d-f) demonstrated a characteristic decline in endurance, strength and motor coordination. However, L2-Cmu treatment in females mitigated the age-related decline in exercise tolerance (FIG. 3a; P≤0.05), grip strength (FIG. 3b; P≤0.05), and balance, by reducing the number of slips on a medium and hard difficulty balance beam (FIG. 3c; P≤0.05). In males, declining exercise tolerance was modestly mitigated with mAb treatment (˜28%; FIG. 3d; P≤0.05), but no effect was observed on strength (FIG. 3e), and coordination was only marginally improved on a medium difficulty beam (FIG. 3f; P≤0.05).

IGF-1R Modulation Improves Female Cardiac Function

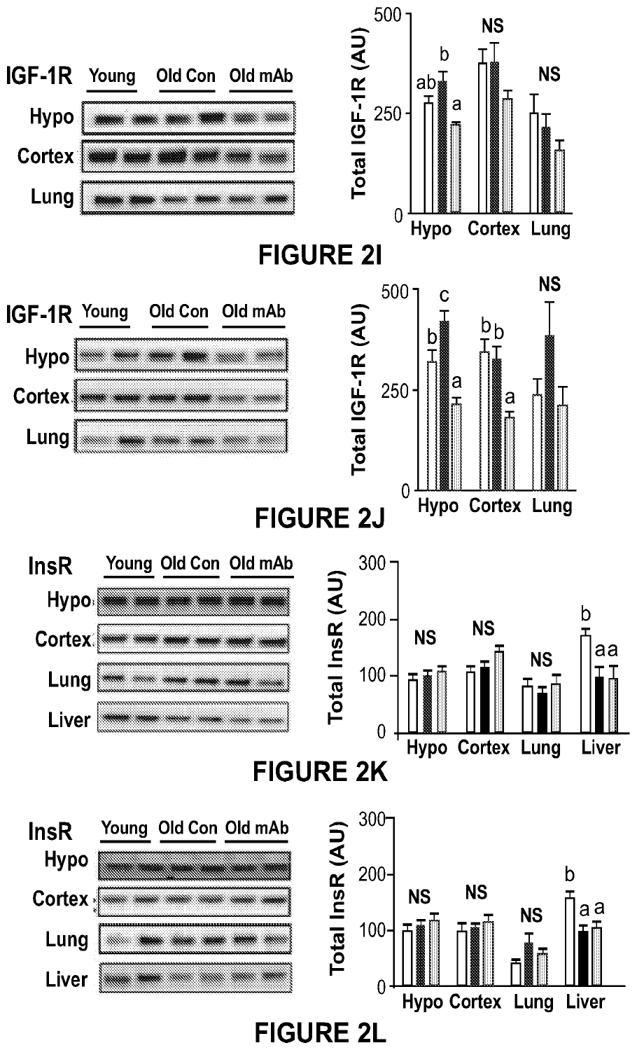

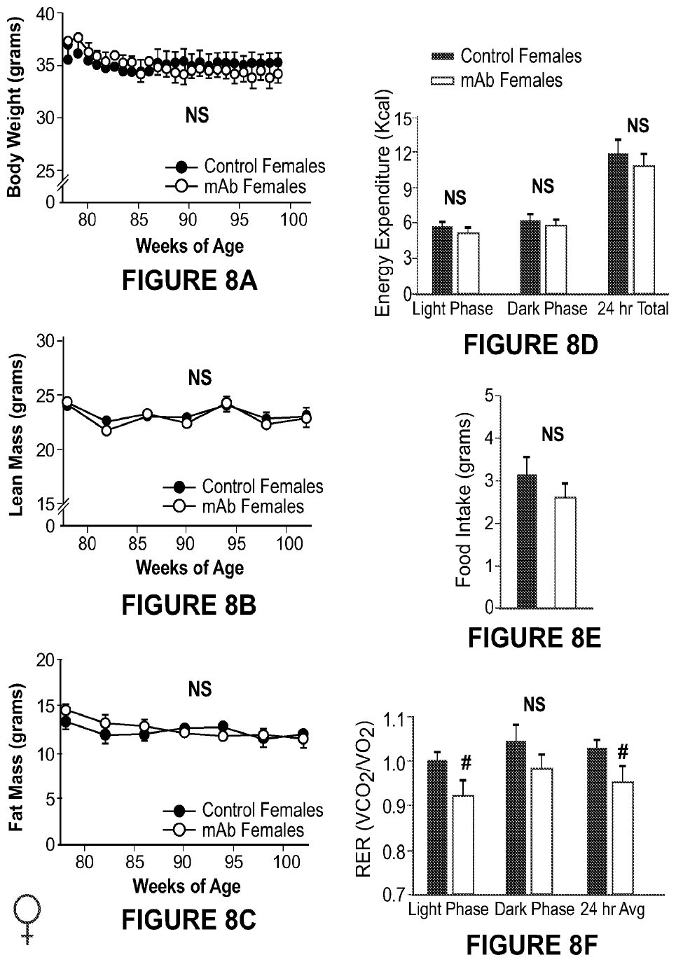

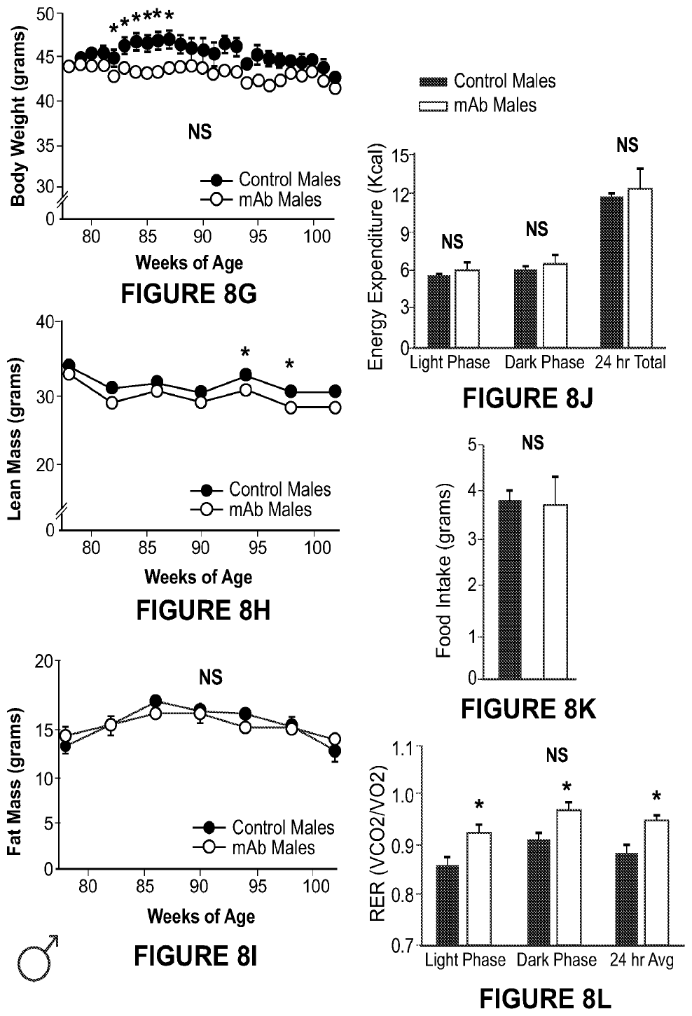

Given the reported importance of IGF-1 signaling to the myocardium^{23,24}, we next assessed the effects of IGF-1R antagonism on cardiovascular function by echocardiography (FIG. 4). Cardiac aging in mice is characterized by a decline in diastolic function²⁵, which we confirmed by a reduction in the E/A ratio in both sexes (FIG. 4a,e; P<0.05). Importantly, L2-Cmu treatment in females did not adversely affect cardiac function, but instead restored diastolic function to more youthful levels (FIG. 4a), and this was associated with a reduction in measures of left ventricular posterior wall end diastole (LVPWd; FIG. 4b) and cardiac fibrosis (FIG. 4c; P<0.05). However, unlike a recent report where constitutive loss of IGF-1Rs specifically in the myocardium of male mice prevented age-related alterations to the myocardium²⁶, late-life mAb treatment failed to preserve or restore these same parameters in male animals (FIG. 4e-g). Because rapamycin, which extends mouse lifespan²⁷, can reverse age-related diastolic dysfunction and restore a more youthful metabolome in the aged myocardium²⁸, we next evaluated metabolomic changes in Young, Old Con and Old mAb female treated hearts. We observed that the aging heart was predominantly characterized by a rise in the level of glycerophospholids, and to lesser extent, acylcarnitines (Table E6; P<0.05). However, mAb treatment led to a significant alteration in 40 out of 184 measured metabolites (27 after FDR correction), including a reduction in the age-related rise of several glycerophospholipids, resulting in a more youthful metabolomic signature in heart (FIG. 4d; FIG. 12; P<0.05). In contrast, age-related increases in LV mass, heart weight, along with reductions in ejection fraction (EF) and fractional shortening (FS), were unaffected by mAb treatment in either sex (FIG. 11).

TABLE E6
Biocrates Targeted Metabolomics in Female Heart.

| Metabolite | | | | |
|---|---|---|---|---|
| (uM) | Class | Young | Old Control | Old mAb |
| Ala | aminoacids | 2039.4 ± 82.3 | 2144.5 ± 106.7 | 1870.0 ± 101.9 |
| Arg | aminoacids | 136.2 ± 8.4^{a} | 91.0 ± 4.6^{b} | 95.6 ± 8.3^{b} |
| Asn | aminoacids | 174.3 ± 8.1 | 188.6 ± 10.4 | 175.7 ± 6.5 |
| Asp | aminoacids | 1194.9 ± 197.7 | 995.0 ± 132.8 | 1089.0 ± 155.3 |
| Cit | aminoacids | 4.6 ± 4.3 | 3.2 ± 2.7 | 0.0 ± 0.0 |
| Gln | aminoacids | 5968.6 ± 256.0 | 5960.0 ± 368.2 | 5697.5 ± 418.7 |
| Glu | aminoacids | 3522.9 ± 230.2 | 3334.5 ± 219.1 | 3506.0 ± 259.4 |
| Gly | aminoacids | 397.6 ± 42.2^{a} | 492.9 ± 67.0 | 339.8 ± 21.6 |
| His | aminoacids | 212.8 ± 7.1 | 223.0 ± 8.6 | 200.3 ± 14.0 |
| Ile | aminoacids | 27.3 ± 19.8 | 27.8 ± 11.8 | 48.3 ± 19.3 |
| Leu | aminoacids | 4.1 ± 3.8 | 0.0 ± 0.0 | 24.0 ± 17.6 |
| Lys | aminoacids | 544.0 ± 27.5 | 533.5 ± 39.6 | 490.0 ± 21.2 |
| Met | aminoacids | 76.7 ± 5.9 | 84.0 ± 9.3 | 71.1 ± 6.4 |
| Orn | aminoacids | 10.5 ± 3.6 | 4.9 ± 3.4 | 0.0 ± 0.0 |
| Phe | aminoacids | 57.2 ± 2.3 | 67.0 ± 5.2 | 61.5 ± 4.5 |
| Pro | aminoacids | 88.3 ± 2.7 | 100.6 ± 9.0 | 83.5 ± 6.7 |
| Ser | aminoacids | 355.3 ± 14.8 | 382.2 ± 25.2 | 292.5 ± 16.2 |
| Thr | aminoacids | 353.7 ± 12.4 | 469.9 ± 40.9 | 422.8 ± 31.3 |
| Trp | aminoacids | 36.8 ± 2.3 | 34.7 ± 3.8 | 37.8 ± 3.0 |
| Tyr | aminoacids | 65.1 ± 3.9 | 71.4 ± 6.2 | 65.2 ± 5.6 |
| Val | aminoacids | 110.5 ± 4.3^{a} | 130.1 ± 4.9^{b} | 116.5 ± 6.1^{ab} |
| Ac-Orn | biogenic amines | ND | ND | ND |
| ADMA | biogenic amines | ND | ND | ND |
| alpha-AAA | biogenic amines | ND | ND | ND |
| c4-OH-Pro | biogenic amines | ND | ND | ND |
| Carnosine | biogenic amines | 7.3 ± 0.4 | 4.0 ± 0.5 | 3.9 ± 0.4 |
| Creatinine | biogenic amines | 153.1 ± 7.6 | 170.6 ± 8.3 | 152.2 ± 5.9 |
| DOPA | biogenic amines | ND | ND | ND |
| Dopamine | biogenic amines | ND | ND | ND |
| Histamine | biogenic amines | 11.7 ± 1.3 | 12.6 ± 0.7 | 12.4 ± 0.9 |
| Kynurenine | biogenic amines | 0.0 ± 0.0 | 0.1 ± 0.1 | 0.0 ± 0.0 |
| Met-SO | biogenic amines | ND | ND | ND |
| Nitro-Tyr | biogenic amines | ND | ND | ND |
| PEA | biogenic amines | ND | ND | ND |
| Putrescine | biogenic amines | 3.8 ± 0.9 | 5.7 ± 2.1 | 4.6 ± 1.2 |
| Sarcosine | biogenic amines | 0.0 ± 0.0 | 0.0 ± 0.0 | 0.0 ± 0.0 |
| Serotonin | biogenic amines | 9.1 ± 3.2 | 5.9 ± 3.6 | 6.3 ± 2.7 |
| Spermidine | biogenic amines | 4.6 ± 0.3 | 5.1 ± 0.4 | 4.6 ± 0.2 |
| Spermine | biogenic amines | 1.5 ± 0.2 | 2.1 ± 0.3 | 1.6 ± 0.2 |
| t4-OH-Pro | biogenic amines | 15.3 ± 1.2^{a} | 10.6 ± 0.6^{b} | 10.2 ± 0.6^{b} |
| Taurine | biogenic amines | 4363.4 ± 22.6 | 4425.5 ± 36.7 | 4326.0 ± 19.3 |
| SDMA | biogenic amines | ND | ND | ND |
| C0 | acylcarnitines | 277.1 ± 11.2 | 283.0 ± 15.5 | 237.2 ± 20.8 |
| C10 | acylcarnitines | 0.8 ± 0.1 | 0.8 ± 0.1 | 0.9 ± 0.1 |
| C10:1 | acylcarnitines | 0.8 ± 0.0 | 0.7 ± 0.1 | 0.7 ± 0.0 |
| C10:2 | acylcarnitines | 0.0 ± 0.0 | 0.1 ± 0.0 | 0.0 ± 0.0 |
| C12 | acylcarnitines | 1.7 ± 0.2 | 1.8 ± 0.2 | 2.2 ± 0.4 |
| C12-DC | acylcarnitines | 0.118 ± 0.019 | 0.116 ± 0.004 | 0.127 ± 0.012 |
| C12:1 | acylcarnitines | 1.0 ± 0.0 | 0.9 ± 0.1 | 1.0 ± 0.1 |
| C14 | acylcarnitines | 3.8 ± 0.5 | 4.7 ± 0.7 | 5.5 ± 1.1 |
| C14:1 | acylcarnitines | 0.7 ± 0.1 | 1.0 ± 0.1 | 1.3 ± 0.3 |
| C14:1-OH | acylcarnitines | 0.3 ± 0.0 | 0.4 ± 0.0 | 0.6 ± 0.1 |
| C14:2 | acylcarnitines | 0.2 ± 0.0 | 0.3 ± 0.0 | 0.4 ± 0.1 |
| C14:2-OH | acylcarnitines | 0.1 ± 0.0 | 0.1 ± 0.0 | 0.1 ± 0.0 |
| C16 | acylcarnitines | 9.3 ± 1.2 | 12.5 ± 2.1 | 14.1 ± 3.0 |
| C16-OH | acylcarnitines | 0.9 ± 0.1 | 1.2 ± 0.2 | 1.3 ± 0.3 |
| C16:1 | acylcarnitines | 4.4 ± 0.5 | 6.9 ± 1.0 | 8.4 ± 1.8 |
| C16:1-OH | acylcarnitines | 0.7 ± 0.1 | 1.0 ± 0.1 | 1.2 ± 0.2 |
| C16:2 | acylcarnitines | 0.9 ± 0.1 | 1.3 ± 0.2 | 1.8 ± 0.4 |
| C16:2-OH | acylcarnitines | 0.2 ± 0.0 | 0.3 ± 0.0 | 0.3 ± 0.1 |
| C18 | acylcarnitines | 7.3 ± 0.9 | 9.6 ± 1.3 | 10.0 ± 2.1 |
| C18:1 | acylcarnitines | 12.1 ± 1.3^{a} | 20.9 ± 3.3^{b} | 26.6 ± 5.9^{b} |
| C18:1-OH | acylcarnitines | 1.8 ± 0.2^{a} | 3.0 ± 0.5^{b} | 3.6 ± 0.7^{b} |
| C18:2 | acylcarnitines | 3.1 ± 0.3^{a} | 5.0 ± 0.8^{ab} | 6.8 ± 1.5^{b} |
| C2 | acylcarnitines | 226.7 ± 8.3 | 221.3 ± 10.5 | 214.5 ± 11.3 |
| C3 | acylcarnitines | 6.5 ± 0.4 | 5.8 ± 0.8 | 4.8 ± 0.5 |
| C3-DC | acylcarnitines | 12.5 ± 0.9 | 12.3 ± 1.2 | 11.1 ± 0.7 |
| (C4-OH) | | | | |
| C3-OH | acylcarnitines | ND | ND | ND |
| C3:1 | acylcarnitines | ND | ND | ND |
| C4 | acylcarnitines | 16.7 ± 0.9 | 16.4 ± 1.1 | 15.0 ± 0.7 |
| C4:1 | acylcarnitines | 0.2 ± 0.0 | 0.2 ± 0.0 | 0.2 ± 0.0 |
| C6 (C4:1-DC) | acylcarnitines | 2.9 ± 0.2 | 2.9 ± 0.2 | 2.8 ± 0.2 |
| C5 | acylcarnitines | 1.1 ± 0.1 | 1.0 ± 0.1 | 1.0 ± 0.1 |
| C5-M-DC | acylcarnitines | 0.1 ± 0.0 | 0.1 ± 0.0 | 0.1 ± 0.0 |
| C5-OH | acylcarnitines | 2.2 ± 0.1 | 2.2 ± 0.1 | 1.9 ± 0.1 |
| (C3-DC-M) | | | | |
| C5:1 | acylcarnitines | 0.2 ± 0.0 | 0.1 ± 0.0 | 0.1 ± 0.0 |
| C5:1-DC | acylcarnitines | 0.2 ± 0.0 | 0.2 ± 0.0 | 0.1 ± 0.0 |
| C5-DC | acylcarnitines | 0.8 ± 0.0 | 0.7 ± 0.1 | 0.6 ± 0.1 |
| (C6-OH) | | | | |
| C6:1 | acylcarnitines | 0.1 ± 0.0 | 0.1 ± 0.0 | 0.1 ± 0.0 |
| C7-DC | acylcarnitines | 0.2 ± 0.0 | 0.2 ± 0.0 | 0.2 ± 0.0 |
| C8 | acylcarnitines | 0.8 ± 0.1 | 0.8 ± 0.1 | 0.9 ± 0.1 |
| C9 | acylcarnitines | ND | ND | ND |
| lysoPC a | glycerophospholipids | 13.7 ± 0.1 | 13.6 ± 0.1 | 13.6 ± 0.1 |
| C14:0 | | | | |
| lysoPC a | glycerophospholipids | 64.7 ± 2.3^{a} | 81.5 ± 2.0^{c} | 72.2 ± 2.2^{b} |
| C16:0 | | | | |
| lysoPC a | glycerophospholipids | 1.7 ± 0.1^{a} | 2.4 ± 0.3^{b} | 2.0 ± 0.1^{b} |
| C16:1 | | | | |
| lysoPC a | glycerophospholipids | 1.1 ± 0.0^{a} | 1.5 ± 0.1^{b} | 1.4 ± 0.1^{b} |
| C17:0 | | | | |
| lysoPC a | glycerophospholipids | 45.0 ± 1.8^{a} | 57.8 ± 1.7^{b} | 54.7 ± 2.9^{b} |
| C18:0 | | | | |
| lysoPC a | glycerophospholipids | 22.5 ± 0.7^{a} | 32.1 ± 3.2^{b} | 27.3 ± 1.5^{b} |
| C18:1 | | | | |
| lysoPC a | glycerophospholipids | 26.1 ± 0.9 | 29.1 ± 2.4 | 26.2 ± 1.1 |
| C18:2 | | | | |
| lysoPC a | glycerophospholipids | 3.3 ± 0.1 | 3.6 ± 0.3 | 3.0 ± 0.2 |
| C20:3 | | | | |
| lysoPC a | glycerophospholipids | 11.0 ± 0.6 | 13.4 ± 1.1 | 14.3 ± 0.4 |
| C20:4 | | | | |
| lysoPC a | glycerophospholipids | 2.3 ± 0.1 | 2.3 ± 0.1 | 2.3 ± 0.1 |
| C24:0 | | | | |
| lysoPC a | glycerophospholipids | 1.2 ± 0.0^{a} | 1.5 ± 0.1^{b} | 1.2 ± 0.1^{a} |
| C26:0 | | | | |
| lysoPC a | glycerophospholipids | 0.5 ± 0.0^{a} | 0.7 ± 0.0^{b} | 0.5 ± 0.0^{a} |
| C26:1 | | | | |
| lysoPC a | glycerophospholipids | 1.0 ± 0.1^{a} | 1.5 ± 0.1^{b} | 1.2 ± 0.1^{a} |
| C28:0 | | | | |
| lysoPC a | glycerophospholipids | 0.7 ± 0.0^{a} | 1.0 ± 0.1^{b} | 0.8 ± 0.1^{a} |
| C28:1 | | | | |
| PC aa C24:0 | glycerophospholipids | 0.45 ± 0.03^{a} | 0.69 ± 0.04^{b} | 0.66 ± 0.05^{b} |
| PC aa C26:0 | glycerophospholipids | 1.5 ± 0.0^{a} | 1.8 ± 0.0^{b} | 1.6 ± 0.1^{a} |
| PC aa C28:1 | glycerophospholipids | 1.2 ± 0.1^{a} | 1.4 ± 0.1^{b} | 1.2 ± 0.1^{a} |
| PC aa C30:0 | glycerophospholipids | 10.2 ± 0.4^{a} | 12.4 ± 0.8^{b} | 9.5 ± 0.4^{a} |
| PC aa C32:0 | glycerophospholipids | 295.5 ± 7.1^{ab} | 324.6 ± 11.7^{b} | 288.9 ± 11.2^{a} |
| PC aa C32:1 | glycerophospholipids | 41.7 ± 1.8^{a} | 58.4 ± 5.3^{b} | 44.2 ± 3.2^{a} |
| PC aa C32:2 | glycerophospholipids | 4.2 ± 0.2^{a} | 5.4 ± 0.5^{b} | 3.9 ± 0.3^{a} |
| PC aa C32:3 | glycerophospholipids | 0.6 ± 0.0^{a} | 0.9 ± 0.0^{b} | 0.7 ± 0.0^{a} |
| PC aa C34:1 | glycerophospholipids | 846.3 ± 26.5^{a} | 1002.0 ± 66.0^{b} | 777.5 ± 63.9^{b} |
| PC aa C34:2 | glycerophospholipids | 524.6 ± 25.0^{a} | 525.1 ± 60.3^{a} | 371.9 ± 29.6^{b} |
| PC aa C34:3 | glycerophospholipids | 15.3 ± 0.7 | 17.9 ± 2.0 | 13.6 ± 1.0 |
| PC aa C34:4 | glycerophospholipids | 1.3 ± 0.0^{a} | 1.7 ± 0.1^{b} | 1.4 ± 0.1^{a} |
| PC aa C36:0 | glycerophospholipids | 230.3 ± 10.5 | 213.4 ± 7.4 | 200.2 ± 8.6 |
| PC aa C36:1 | glycerophospholipids | 229.8 ± 9.4^{a} | 301.6 ± 18.1^{b} | 240.0 ± 17.0^{a} |
| PC aa C36:2 | glycerophospholipids | 373.8 ± 13.7 | 442.6 ± 38.1 | 328.0 ± 27.4 |
| PC aa C36:3 | glycerophospholipids | 180.2 ± 6.4 | 211.1 ± 21.9 | 155.3 ± 14.2 |
| PC aa C36:4 | glycerophospholipids | 598.3 ± 22.9^{a} | 758.0 ± 22.9^{b} | 692.0 ± 38.3^{ab} |
| PC aa C36:5 | glycerophospholipids | 11.7 ± 0.3 | 13.9 ± 1.0 | 12.5 ± 0.8 |
| PC aa C36:6 | glycerophospholipids | 3.6 ± 0.1 | 4.0 ± 0.3 | 3.6 ± 0.3 |
| PC aa C38:0 | glycerophospholipids | 50.7 ± 2.3 | 48.2 ± 3.2 | 49.3 ± 2.0 |
| PC aa C38:3 | glycerophospholipids | 53.6 ± 1.7 | 58.8 ± 3.5 | 46.9 ± 2.9 |
| PC aa C38:4 | glycerophospholipids | 700.0 ± 31.8^{a} | 1053.0 ± 27.2^{b} | 967.5 ± 59.4^{b} |
| PC aa C38:5 | glycerophospholipids | 270.2 ± 11.7 | 321.1 ± 9.2 | 296.7 ± 16.3 |
| PC aa C38:6 | glycerophospholipids | 1914.3 ± 84.6 | 1963.0 ± 102.5 | 1949.5 ± 84.9 |
| PC aa C40:1 | glycerophospholipids | 2.0 ± 0.0 | 2.1 ± 0.1 | 2.1 ± 0.1 |
| PC aa C40:2 | glycerophospholipids | 2.4 ± 0.1^{a} | 3.1 ± 0.1^{b} | 2.7 ± 0.2^{a} |
| PC aa C40:3 | glycerophospholipids | 3.9 ± 0.1^{a} | 5.4 ± 0.4^{b} | 4.4 ± 0.3^{a} |
| PC aa C40:4 | glycerophospholipids | 51.9 ± 2.2^{a} | 64.9 ± 1.8^{b} | 58.3 ± 3.0^{a} |
| PC aa C40:5 | glycerophospholipids | 226.3 ± 13.1 | 235.1 ± 9.5 | 212.7 ± 12.8 |
| PC aa C40:6 | glycerophospholipids | 1772.0 ± 76.4 | 1841.5 ± 85.8 | 1797.0 ± 97.8 |
| PC aa C42:0 | glycerophospholipids | 1.61 ± 0.06^{b} | 1.35 ± 0.10^{a} | 1.30 ± 0.04^{a} |
| PC aa C42:1 | glycerophospholipids | 0.8 ± 0.0 | 0.8 ± 0.0 | 0.8 ± 0.0 |
| PC aa C42:2 | glycerophospholipids | 1.12 ± 0.04^{a} | 1.30 ± 0.04^{b} | 1.15 ± 0.05^{a} |
| PC aa C42:4 | glycerophospholipids | 3.0 ± 0.1^{a} | 3.9 ± 0.2^{b} | 3.2 ± 0.2^{a} |
| PC aa C42:5 | glycerophospholipids | 4.4 ± 0.1 | 4.4 ± 0.2 | 4.1 ± 0.2 |
| PC aa C42:6 | glycerophospholipids | 18.0 ± 0.6 | 18.1 ± 1.5 | 18.2 ± 1.0 |
| PC ae C30:0 | glycerophospholipids | 0.77 ± 0.02^{a} | 0.96 ± 0.03^{b} | 0.81 ± 0.05^{a} |
| PC ae C30:1 | glycerophospholipids | 0.21 ± 0.02^{a} | 0.35 ± 0.16^{b} | 0.26 ± 0.02^{a} |
| PC ae C30:2 | glycerophospholipids | 0.4 ± 0.0 | 0.5 ± 0.0 | 0.4 ± 0.0 |
| PC ae C32:1 | glycerophospholipids | 6.8 ± 0.3^{a} | 9.4 ± 0.5^{c} | 7.9 ± 0.3^{b} |
| PC ae C32:2 | glycerophospholipids | 1.2 ± 0.0^{a} | 1.8 ± 0.1^{b} | 1.4 ± 0.1^{a} |
| PC ae C34:0 | glycerophospholipids | 7.0 ± 0.1 | 7.5 ± 0.3 | 6.9 ± 0.2 |
| PC ae C34:1 | glycerophospholipids | 27.2 ± 0.5^{a} | 34.2 ± 1.5^{b} | 29.1 ± 1.3^{a} |
| PC ae C34:2 | glycerophospholipids | 18.8 ± 0.7^{a} | 22.2 ± 0.8^{b} | 18.2 ± 1.0^{a} |
| PC ae C34:3 | glycerophospholipids | 10.3 ± 0.4^{a} | 10.2 ± 0.9^{a} | 7.9 ± 0.6^{b} |
| PC ae C36:0 | glycerophospholipids | 3.2 ± 0.1^{a} | 3.8 ± 0.2^{b} | 2.9 ± 0.3^{a} |
| PC ae C36:1 | glycerophospholipids | 60.5 ± 2.1^{ab} | 67.8 ± 4.1^{b} | 56.7 ± 3.0^{a} |
| PC ae C36:2 | glycerophospholipids | 15.2 ± 0.6 | 17.6 ± 1.5 | 14.3 ± 0.8 |
| PC ae C36:3 | glycerophospholipids | 7.3 ± 0.3 | 7.9 ± 0.4 | 6.7 ± 0.3 |
| PC ae C36:4 | glycerophospholipids | 14.7 ± 0.3 | 16.8 ± 0.4 | 15.4 ± 0.6 |
| PC ae C36:5 | glycerophospholipids | 33.4 ± 1.3^{a} | 39.6 ± 1.0^{b} | 35.8 ± 1.4^{a} |
| PC ae C38:0 | glycerophospholipids | 20.8 ± 0.9^{a} | 25.7 ± 1.1^{b} | 26.1 ± 1.6^{b} |
| PC ae C38:1 | glycerophospholipids | 15.1 ± 0.4^{a} | 19.2 ± 1.2^{b} | 15.3 ± 0.9^{a} |
| PC ae C38:2 | glycerophospholipids | 8.4 ± 0.4^{a} | 10.8 ± 1.0^{b} | 8.1 ± 0.6^{a} |
| PC ae C38:3 | glycerophospholipids | 7.7 ± 0.3^{a} | 10.1 ± 0.6^{b} | 7.5 ± 0.5^{a} |
| PC ae C38:4 | glycerophospholipids | 17.4 ± 0.6^{a} | 23.6 ± 0.7^{b} | 22.2 ± 1.1^{b} |
| PC ae C38:5 | glycerophospholipids | 25.3 ± 0.9 | 27.9 ± 1.2 | 26.9 ± 1.2 |
| PC ae C38:6 | glycerophospholipids | 79.8 ± 4.0 | 77.1 ± 2.4 | 77.6 ± 3.7 |
| PC ae C40:1 | glycerophospholipids | 270.8 ± 10.8 | 239.2 ± 19.0 | 228.7 ± 18.3 |
| PC ae C40:2 | glycerophospholipids | 6.5 ± 0.2 | 6.6 ± 0.3 | 6.4 ± 0.5 |
| PC ae C40:3 | glycerophospholipids | 3.7 ± 0.1^{a} | 6.0 ± 0.3^{b} | 4.1 ± 0.4^{a} |
| PC ae C40:4 | glycerophospholipids | 8.8 ± 0.3^{a} | 12.6 ± 0.6^{b} | 11.9 ± 0.5^{b} |
| PC ae C40:5 | glycerophospholipids | 14.5 ± 0.7^{a} | 17.9 ± 0.8^{b} | 14.9 ± 0.7^{a} |
| PC ae C40:6 | glycerophospholipids | 37.2 ± 1.6 | 40.2 ± 2.2 | 40.3 ± 1.9 |
| PC ae C42:0 | glycerophospholipids | 12.0 ± 0.4 | 12.6 ± 0.8 | 13.6 ± 0.7 |
| PC ae C42:1 | glycerophospholipids | 9.7 ± 0.5 | 8.8 ± 0.4 | 8.8 ± 0.5 |
| PC ae C42:2 | glycerophospholipids | 6.4 ± 0.3 | 6.5 ± 0.3 | 6.7 ± 0.4 |
| PC ae C42:3 | glycerophospholipids | 7.8 ± 0.3^{a} | 10.4 ± 0.5^{b} | 10.6 ± 0.9^{b} |
| PC ae C42:4 | glycerophospholipids | 1.2 ± 0.1^{a} | 1.9 ± 0.1^{c} | 1.5 ± 0.1^{b} |
| PC ae C42:5 | glycerophospholipids | 9.4 ± 0.5^{a} | 11.6 ± 0.4^{b} | 9.5 ± 0.5^{a} |
| PC ae C44:3 | glycerophospholipids | 2.3 ± 0.1 | 2.2 ± 0.1 | 2.2 ± 0.1 |
| PC ae C44:4 | glycerophospholipids | 2.2 ± 0.1 | 2.2 ± 0.1 | 2.1 ± 0.2 |
| PC ae C44:5 | glycerophospholipids | 4.2 ± 0.2 | 4.5 ± 0.3 | 4.9 ± 0.4 |
| PC ae C44:6 | glycerophospholipids | 0.7 ± 0.0 | 0.8 ± 0.1 | 0.7 ± 0.0 |
| SM (OH) | sphingolipids | 3.7 ± 0.2 | 3.3 ± 0.3 | 3.1 ± 0.1 |
| C14:1 | | | | |
| SM (OH) | sphingolipids | 4.2 ± 0.2 | 4.2 ± 0.2 | 3.8 ± 0.1 |
| C16:1 | | | | |
| SM (OH) | sphingolipids | 16.2 ± 0.6 | 14.6 ± 0.8 | 14.0 ± 0.3 |
| C22:1 | | | | |
| SM (OH) | sphingolipids | 7.2 ± 0.3 | 6.8 ± 0.4 | 6.5 ± 0.2 |
| C22:2 | | | | |
| SM (OH) | sphingolipids | 1.1 ± 0.1 | 1.0 ± 0.1 | 0.9 ± 0.0 |
| C24:1 | | | | |
| SM C16:0 | sphingolipids | 88.8 ± 4.7^{a} | 119.6 ± 9.1^{b} | 102.4 ± 3.8^{b} |
| SM C16:1 | sphingolipids | 3.6 ± 0.2 | 4.2 ± 0.2 | 3.8 ± 0.2 |
| SM C18:0 | sphingolipids | 63.3 ± 3.0^{a} | 78.0 ± 3.9^{b} | 66.2 ± 2.9^{a} |
| SM C18:1 | sphingolipids | 9.1 ± 0.5 | 10.6 ± 0.6 | 9.0 ± 0.4 |
| SM C20:2 | sphingolipids | 0.3 ± 0.0 | 0.3 ± 0.0 | 0.3 ± 0.0 |
| SM C24:0 | sphingolipids | 28.2 ± 1.2 | 25.2 ± 0.9 | 23.4 ± 0.7 |
| SM C24:1 | sphingolipids | 42.4 ± 2.6^{a} | 54.7 ± 2.7^{b} | 50.3 ± 1.8^{b} |
| SM C26:0 | sphingolipids | 0.20 ± 0.02 | 0.22 ± 0.03 | 0.15 ± 0.02 |
| SM C26:1 | sphingolipids | 0.36 ± 0.04 | 0.44 ± 0.03 | 0.38 ± 0.03 |

Data are means ± SE. Different letters denote a significant difference between groups, P < 0.05. Corresponding data Heatmap is shown in FIG. 4d.

IGF-1R Modulation Promotes Resilience to Chemotherapy in Males and Females

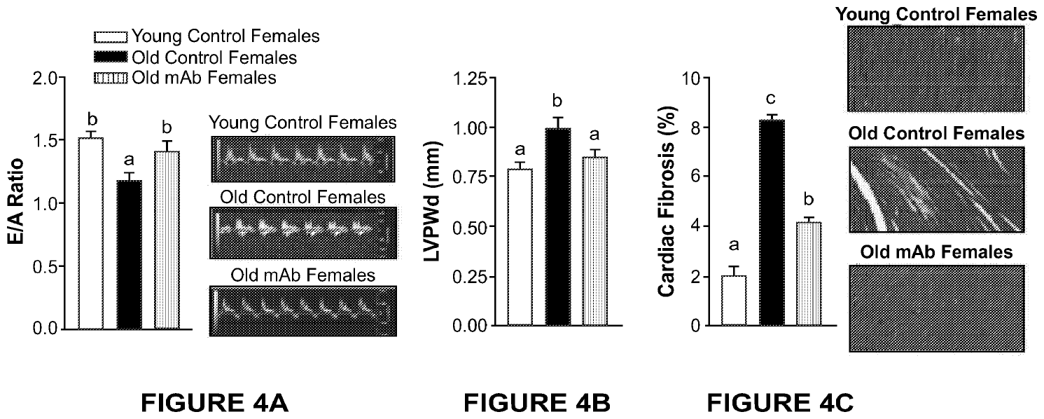

Given the therapeutic benefit of IGF-1R mAb treatment on female cardiac function in normal aging, coupled with reported improvements in stress resistance from reduced IGF-1 signaling^{6,7}, we next determined if IGF-1R modulation could mitigate the well-known cardiotoxic effects of doxorubicin (DOX). Beginning at 15 mo of age, male and female C57BL/6 mice were preemptively treated with vehicle or L2-Cmu for 3 mo. Mice were then evaluated by echocardiogram prior to a low-dose, DOX challenge (4 mg/kg/wk; i.p) for 3 (males) or 4 weeks (females), and then re-assessed for effects on cardiac function. In females, EF and FS were reduced while left ventricular posterior wall end systole (LVPWd) was increased following DOX treatment, but these changes were all mitigated by mAb treatment (FIG. 5a-c; P<0.05). Similarly, EF, FS, and LVPWd were all reduced by DOX in male Controls (FIG. 5i-j,l; P<0.05) and mAb effectively prevented these adverse changes. Furthermore, when frailty symptoms were evaluated after DOX treatment, both mAb-treated females and males were less frail than Controls, suggesting improved whole organismal resilience to DOX (FIG. 5f, n; P<0.05). Thus, while the impact of mAb treatment on several parameters of normal aging are sex dependent, its ability to improve resilience to a chemotherapeutic stressor was similarly effective in both sexes.

Sexually-Dimorphic Effects of IGF-1R Modulation on Inflammatory and Stress and/or Senescence Markers

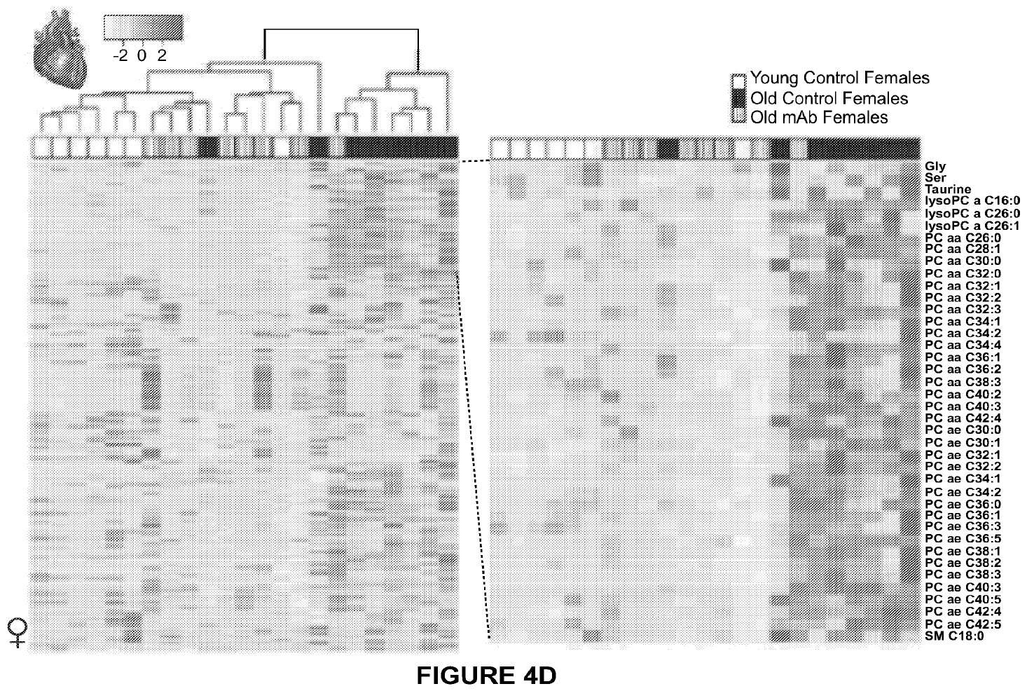

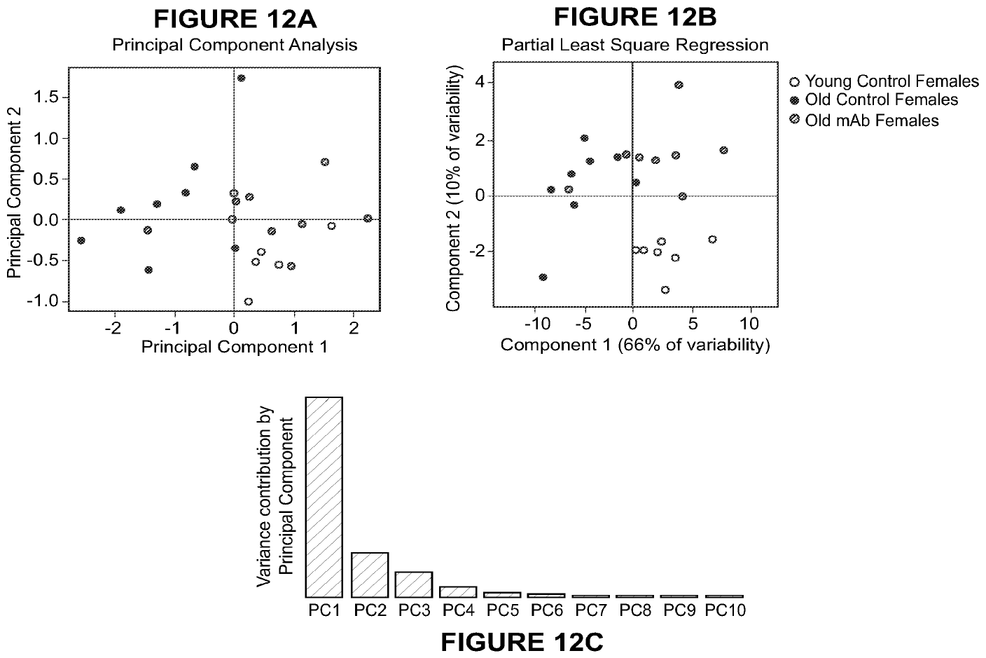

A rise in pro-inflammatory mediators is a hallmark of aging, thus we evaluated plasma inflammatory markers using a 25-plex immunoassay to determine if these parameters were affected by mAb treatment in male and female mice. Aging in females was characterized by a significant rise in IL-1β, IL-4, IL-5, IL-6, IL-10, IL-12(p40), IL-12(p70), IL-17, CXCL-10, CXCL-1, MIP-1α, MIP-2, and TNFα, and several of these cytokines and chemokines were restored to a more youthful level with mAb treatment (Table E7; P<0.05). In contrast, only G-CSF, IL-6 and RANTES were elevated in old male plasma, but mAb treatment led to a marked increase in markers shown in Table E8, demonstrating a clear exacerbation of systemic inflammatory status in male mice. Furthermore, mAb treatment led to less p16 expression in female lung (FIG. 6c), but not in male lung (FIG. 6d), though no significant effect of mAb treatment was observed on NF-κB activation in several tissues (FIG. 13).

TABLE E7
Inflammatory cytokines and chemokines in female mice.

| | Young | Old Con | Old mAb |
|---|---|---|---|
| Analyte | (n = 8) | (n = 15) | (n = 16) |
| G-CSF | 283.2 ± 31.5^{a} | 917.9 ± 437.7^{ab} | 265.4 ± 117.5^{b} |
| GM-CSF | 5.5 ± 0.0 | 25.6 ± 6.8 | 16.5 ± 7.8 |
| IFNγ | 0.6 ± 0.0 | 6.3 ± 2.4 | 7.0 ± 6.5 |
| IL-1 a | 84.7 ± 14.4 | 107.3 ± 12.7 | 138.4 ± 45.5 |
| IL-1β | 2.7 ± 0.0^{a} | 707.5 ± 636.4^{b} | 69.6 ± 60.9^{ab} |
| IL-2 | 0.5 ± 0.0 | 6.1 ± 2.4 | 1.3 ± 0.8 |
| IL-4 | 0.2 ± 0.0^{a} | 213.1 ± 95.6^{b} | 24.5 ± 24.3^{a} |
| IL-5 | 4.4 ± 2.4^{a} | 345.1 ± 200.6^{b} | 57.1 ± 53.5^{a} |
| IL-6 | 0.6 ± 0.0^{a} | 1134.8 ± 560.0^{b} | 38.3 ± 36.0^{a} |
| IL-7 | 0.7 ± 0.0^{a} | 46.2 ± 39.4^{ab} | 151.0 ± 57.9^{b} |
| IL-9 | ND | ND | ND |
| IL-10 | 1.0 ± 0.0^{a} | 2284.4 ± 1191.0^{b} | 81.6 ± 52.1^{b} |
| IL-12(p40) | 2.5 ± 0.6^{a} | 41.9 ± 20.0^{b} | 5.8 ± 2.1^{a} |
| IL-12(p70) | 2.4 ± 0.0^{a} | 2485.2 ± 1190.0^{b} | 139.8 ± 107.0^{ab} |
| IL-13 | 62.1 ± 8.6 | 181.3 ± 67.5 | 69.2 ± 19.0 |
| IL-15 | 3.7 ± 0.0^{a} | 300.2 ± 282.9^{ab} | 1035.5 ± 413.5^{b} |
| IL-17 | 0.25 ± 0.0^{a} | 549.1 ± 251.6^{b} | 15.0 ± 13.0^{ab} |
| CXCL-10 | 99.3 ± 8.9^{a} | 209.6 ± 78.2^{b} | 327.1 ± 202.3^{ab} |
| CXCL-1 | 27.5 ± 5.8^{a} | 67.8 ± 14.3^{b} | 51.4 ± 19.8^{a} |
| MCP-1 | 3.4 ± 0.0^{a} | 248.3 ± 187.0^{b} | 69.4 ± 38.6^{ab} |
| MIP-1α | 28.2 ± 10.2 | 63.8 ± 13.5^{b} | 14.5 ± 7.5^{a} |
| MIP-1β | 6.0 ± 0.0 | 20.5 ± 8.9 | 48.6 ± 27.8 |
| MIP-2 | 39.8 ± 5.9^{a} | 157.0 ± 43.8^{b} | 63.2 ± 13.8^{a} |
| RANTES | 5.2 ± 1.6 | 775.8 ± 474.8 | 35.6 ± 21.0 |
| TNFα | 1.2 ± 0.0^{a} | 66.1 ± 53.2^{b} | 13.9 ± 7.4^{ab} |

Data are means ± SE. Corresponding log-transformed data Heatmap is shown in FIG. 5a. Data were analyzed by the Kruskal-Wallis procedure and the Mann-Whitney U test when appropriate. Any value below the lower limit of detection of the assay was replaced by the minimal detectable concentration (MOD)/√2 for the specific analyte, and these values were ranked as a tie for purposes of the statistical analysis. Different letters denote a significant difference between groups, P ≤ 0.05.

TABLE E8
Inflammatory cytokines and chemokines in male mice.

| | Young | Old Con | Old mAb |
|---|---|---|---|
| Analyte | (n = 8) | (n = 14) | (n = 16) |
| G-CSF | 179.8 ± 21.0^{a} | 128.9 ± 48.4^{b} | 174.2 ± 73.4^{b} |
| GM-CSF | 6.5 ± 1.0^{a} | 9.1 ± 2.4^{a} | 37.4 ± 7.7^{b} |
| IFNy | 3.7 ± 1.8^{a} | 6.4 ± 2.9^{a} | 36.9 ± 7.8^{b} |
| IL-1α | 31.2 ± 7.2^{a} | 32.0 ± 6.0^{a} | 116.2 ± 50.9^{b} |
| IL-1β | 2.2 ± 0.3^{a} | 8.8 ± 6.3^{a} | 96.0 ± 79.2^{b} |
| IL-2 | 0.5 ± 0.0^{a} | 0.7 ± 0.2^{ab} | 7.3 ± 3.3^{b} |
| IL-4 | 0.2 ± 0.0^{a} | 2.8 ± 2.5^{a} | 16.0 ± 11.1^{b} |
| IL-5 | 1.0 ± 0.2^{a} | 6.6 ± 5.1^{a} | 41.3 ± 18.8^{b} |
| IL-6 | 0.6 ± 0.1^{a} | 4.5 ± 1.9^{b} | 33.9 ± 15.3^{b} |
| IL-7 | 2.1 ± 0.5^{a} | 163.6 ± 189.6^{a} | 218.3 ± 202.6^{b} |
| IL-9 | 76.5 ± 18.4 | 57.5 ± 12.0 | 736.8 ± 665.0 |
| IL-10 | 1.0 ± 0.3^{a} | 3.4 ± 1.7^{a} | 58.5 ± 29.3^{b} |
| IL-12(p40) | 7.1 ± 1.2^{a} | 6.7 ± 2.4^{a} | 47.3 ± 29.5^{b} |
| IL-12(p70) | 2.7 ± 0.3^{a} | 15.1 ± 11.3^{a} | 196.9 ± 85.5^{b} |
| IL-13 | 3.3 ± 0.4 | 3.9 ± 0.6 | 28.7 ± 13.4 |
| IL-15 | 7.1 ± 1.8^{a} | 8.5 ± 3.1^{a} | 81.3 ± 35.3^{b} |
| IL-17 | 0.5 ± 0.1^{a} | 2.1 ± 1.1^{a} | 39.3 ± 19.1^{b} |
| CXCL-10 | 30.5 ± 3.8^{a} | 57.9 ± 7.4^{ab} | 72.1 ± 8.8^{b} |
| CXCL-1 | 28.6 ± 11.7 | 25.9 ± 2.4 | 42.6 ± 7.2 |
| MCP-1 | 10.5 ± 3.4^{a} | 17.0 ± 6.9^{a} | 95.7 ± 37.2^{b} |
| MIP-1α | 31.4 ± 5.7^{a} | 26.4 ± 3.5^{a} | 79.5 ± 14.5^{b} |
| MIP-1β | 18.3 ± 2.5^{a} | 8.0 ± 1.5^{b} | 24.8 ± 6.5^{ab} |
| MIP-2 | 28.7 ± 6.4^{a} | 60.0 ± 15.3^{a} | 157.1 ± 36.8^{b} |
| RANTES | 1.2 ± 0.1^{a} | 2.5 ± 0.8^{b} | 11.1 ± 6.8^{c} |
| TNFα | 6.3 ± 0.5^{a} | 8.2 ± 1.9^{a} | 30.3 ± 7.5^{b} |

Data are means ± SE. Corresponding log-transformed data Heatmap is shown in FIG. 5b. Different letters denote a significant difference between groups, P ≤ 0.05.

Modulation of IGF-1R Reduces Cancer and Improves Longevity or Survival in Females

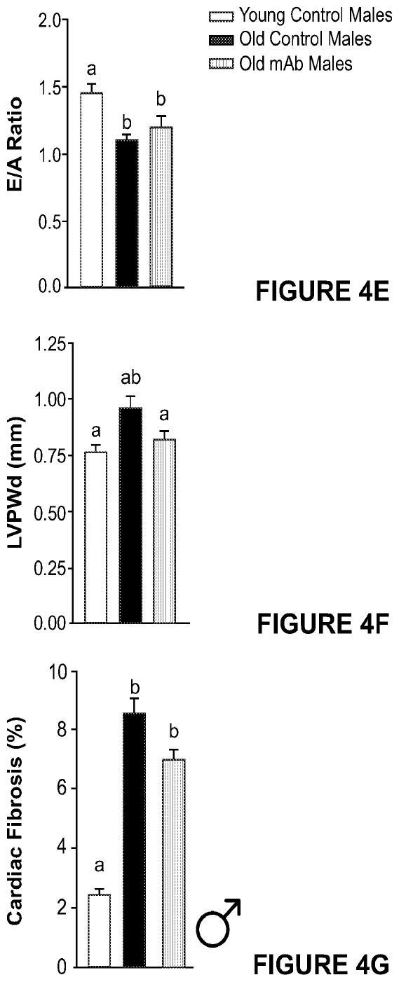

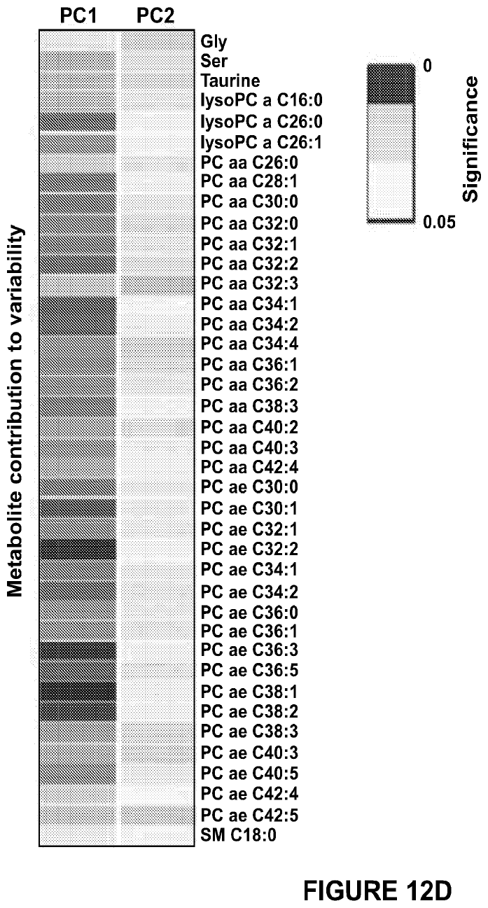

In a 6 mo interim intervention trial with L2-Cmu in older mice, we performed an extensive histopathologic analysis and noted a reduction in endometrial hyperplasia severity (FIG. 14a; n=16 group; P<0.05) and a suggestion of better survival in female mice treated with L2-Cmu (FIG. 14b; n=24 Controls, n=36 mAb; P=0.14). Meanwhile, tumor burden in males tended to be increased (FIG. 14c; P=0.07), and survival to 24 mo with L2-Cmu was indistinguishable from control animals (FIG. 14d; n=36 Controls; n=38 mAb; P=0.77). Thus, to definitively determine if late-life pharmacologic modulation of IGF-1R signaling could improve survival, we performed a longevity study in female mice with lifelong i.p. injections of L2-Cmu once per week, beginning at 18 mo of age until death. As can be observed in this larger cohort (n=45 group), long-term L2-Cmu treatment significantly reduced female body weight (FIG. 7a; P=0.055) and lean body mass (FIG. 7b; P=0.052), with no effect on adiposity (FIG. 7c). Importantly, late-life L2-Cmu treatment improved female survival (FIG. 7d), and the risk of death with late-life L2-Cmu treatment was 62.2% (40.7%, 95.2%) of that observed for controls (P=0.029). Furthermore, end-of-life pathology confirmed that deaths due to cancer were significantly reduced, while those attributable to unknown causes were increased by mAb treatment (Table E9; P<0.05), an observation that is consistent with pathologic assessments in caloric restricted and long-lived GHRKO mice²⁹. However, despite improved survival and less cancers with mAb treatment, no significant effect was observed on maximum lifespan (P=0.971).

TABLE E9
Cause of death in female mice

| | Con | mAb |
|---|---|---|
| | Females | Females |
| Cause | (n = 30) | (n = 20) |
| Neoplasm | 25 (83.3%) | 11 (55%)* |
| Lymphoma | 12 | 10 |
| Lymphoma + other tumor | 4 | 1 |
| Adenocarcinoma | 5 | 0 |
| Pituitary adenoma | 3 | 0 |
| Other tumors | 1 | 0 |
| Non-neoplasm | 5 (16.7%) | 9 (45%) |
| Glomerulonephritis | 1 | 0 |
| Unknown | 4 | 9* |

*Significantly different from Controls, P < 0.05

Discussion

Across nature, diminished growth factor signaling is linked to improved longevity¹². Importantly, this relationship is relevant to humans as individuals with exceptional longevity are enriched with functional IGF-1R mutations¹³, while low IGF-1 levels predict better survival in female nonagenarians¹⁶. Given the clear relationship between IGF-1 and aging, we reasoned that IGF-1R mAbs could provide a translational tool to mimic the beneficial effects reported by reduced signaling in this pathway. Here, we provide the first evidence of improved healthspan and age-related survival with a therapeutic mAb. Indeed, we observed that treatment with an IGF-1R antagonist in older mice significantly and preferentially improved several indices of healthspan as well as lifespan in females, in part by reducing death from neoplastic disease. Similar to rapamycin²⁷, these effects were achieved even though not initiated until later in life, which we reasoned to be a safer therapeutic window for IGF-1R modulation than younger ages³⁰. Therefore, given that IGF-1R mAbs have already been successfully employed in human trials, these drugs could be immediately repurposed to target aging in older humans³¹.

In agreement with previous evidence from genetic models, these data show that chronic modulation of IGF-1R signaling may be most well suited for targeting aging in females⁶⁻⁸, rather than males. Such an indication is unique from most other drugs and compounds identified by the NIA-supported Intervention Testing Program (ITP) to improve lifespan, as various agents, including acarbose, 17-α-estradiol, nondihydroguaiaretic acid (NDGA), and protandim, all preferentially improve male lifespan^{32,33}. The potential explanation for sex differences in the IGF-1 signaling pathway on aging as well as response to other age-delaying interventions is unclear, but unique interactions of candidate pathways and targets with sex hormones, as well as differences in the way drugs are absorbed and metabolized between males and females, could explain in part these differences³⁴.

L2-Cmu treatment resulted in profound sex differences on the circulating inflammatory profile in aged mice, suggesting divergent requirements for IGF-1 signaling on immune cell homeostasis as one possible contributor to this sex dimorphism. Indeed, it was recently shown that IGF-1R ablation from myeloid cells led to dysregulated activation of macrophages³⁵, while IGF-1 signaling has also been linked to regulation of natural killer cells³⁶, Tregs³⁷ and neutrophils²², though sex differences in these responses have not been carefully investigated. In spite of these female-centric benefits on normal aging, L2-Cmu improved stress resistance to a chemotherapeutic challenge in both sexes, suggesting a potential role to improve resilience that is independent of sex differences in this pathway on aging per se.

In summary, these data support the rationale of exploring the potential repurposing of IGF-1R mAbs to target aging in humans. While the optimal therapeutic window to intervene will require further investigation, we reason that starting later in life, a time in which function of the somatrophic axis is diminished, is the best approach. Indeed, administering IGF-1R mAbs in cancer trials was reported to result in increased adverse events in younger populations³⁰, possibly due in part to disruption of the somatotropic axis and associated deleterious effects of excess GH^{38,39}. We did not observe a significant differences in circulating IGF-1 levels with mAb treatment in older mice, and glucose homeostasis was not adversely affected in either sex, suggesting this therapeutic window was appropriate. Nevertheless, further optimization of dose and duration with mAb treatment is warranted, particularly given recent evidence that transient administration of other drugs can have persistent effects on age-related outcomes⁴⁰. Finally, these data uniquely provide a striking example of an intervention which favors female healthspan and survival, thereby reinforcing the need for considering sex differences in devising therapeutic strategies to treat aging and its diseases.

Methods

L2-Cmu Development and Validation

To target IGF-1R action in mice, we utilized the mAb, L2-Cmu (Amgen Inc, Thousand Oaks, Calif.), which is a murinized IgG₁ version of the fully human L2-C mAb previously reported by Calzone et al.²¹. Validation of L2-Cmu was confirmed by Biacore analysis and in murine fibroblasts (NIH-3T3). For NIH-3T3 experiments, cells were grown in DMEM plus 10% fetal bovine serum (Invitrogen). At t=−4 hr, cells were serum starved and at t=−1 hr, pre-treated with vehicle or L2-Cmu (100 ug/mL). Vehicle or IGF-1 (5 nM) was then added to the media for 2 min and cells were then rapidly lysed in ice-cold buffer to assess receptor activation as described below.

Animals

Young (4 mo) and old (18 mo) male and female CB6F1 mice were obtained from the NIA Aged Rodent Colony. All animals were housed at standard temperature (˜22° C.) and humidity-controlled conditions under a 14L:10D photoperiod and provided ad libitum access to water and a low-fat purified diet upon arrival (10% calories from fat D12450H Research Diets Inc). All experiments were approved by the Institutional Animal Care and Use Committee at the Albert Einstein College of Medicine.

Experimental Design and Treatment

CB6F1 mice were assigned to receive either weekly i.p. injections of vehicle or L2-Cmu (20 mg/kg) once per week and were either monitored for interim survival to 24 mo of age (n=24-38 per group) and sacrificed for blood and tissue analyses (n=8-16 per group), or treated until natural death for longevity (females only; n=45 group). Animals deemed severely moribund and anticipated to not survive another 24 hrs were immediately euthanized and this was considered the time of death. In addition, histopathology was conducted at 24 mo (n=16 per group) or end of life (n=20-30 per group), as described below.

Metabolic Phenotyping

Body weight was monitored on a weekly basis and body composition was assessed at 3 mo intervals by qMR (ECHO MRS; Echo Medical Systems). Glucose tolerance tests (GTT) and insulin tolerance tests (ITT) were performed as described“. For GTTs, animals were fasted for 4 hrs and a baseline blood glucose measurement was made prior to administering a 2 mg/kg i.p. glucose injection. Blood glucose was subsequently monitored at 15, 30, 60, 90 and 120 min post injection with a glucose meter (Bayer Contour). ITTs were performed in random-fed mice, early in their light cycle (˜0700h-0800h), as described”. Following a baseline glucose measurement, mice were injected IP with 0.75 U/kg insulin and blood glucose was measured at 15, 30, 45 and 60 min later.

Energy expenditure, substrate utilization food intake and spontaneous activity were determined as described^{42,43}, based upon O₂ consumption and CO₂ production, using a Mouse CLAMS System (Columbus Instruments, Columbus, Ohio). In brief, animals (n=8 per group) were placed into individual cages at their standard temperature and photoperiod and allowed to acclimate for at least 72 hrs prior to the experiment. Beginning at 700h, data were collected over a >24 hr period, and at 1700h the next day, animals were fasted overnight and then refed 19 hrs later (1200h) to evaluate metabolic fuel switching.

Functional Healthspan Assessments

At 23-24 mo of age, motor coordination, strength and endurance were evaluated in mice using a battery of healthspan assessments. Neuromuscular function was determined via balance beam. In brief, animals were first familiarized with walking across a 4 ft plank prior to testing three round beams of increasing difficulty (1″ easy; 0.75″ medium, 0.5″ difficult), with light and food cues as motivation to cross, and the number of slips were counted while transversing the beam⁴⁴. Forelimb grip strength was determined by allowing animals to clasp a suspended wire and the time to release was recorded. Exercise capacity was determined by a single maximal exercise test to voluntary fatigue on a motorized treadmill (Exer 3/6, Columbus Instruments). Mice were first familiarized to the treadmill for 3 non consecutive days for 5 min at a walking speed (8m/min). Animals were then challenged with a graduated fatigue test, beginning at 10 m/min and 4% grade for 3 min, and increasing in speed by 2 m/min every 2 min to a max speed of 16m/min until exhaustion. All tests included a young (4 mo) control group for reference.

Cardiovascular Phenotyping

Systolic and diastolic function was evaluated following 5-6 mo of treatment, as described^{42,43}. In brief, mouse electrocardiography was measured with visual sonic Vevo2100 imaging system (FUJIFILM VisualSonics Inc, Toronto, ON). Cardiac left ventricular dimensions were obtained under M-mode, left ventricle ejection fraction (EF) and fractional shortening (FS) were calculated accordingly. Left ventricular diastolic function presented as the E/A ratio was generated based on transmitral blood flow measured under Color Doppler mode. At sacrifice, heart tissue was immediately harvested, and the heart was perfuse fixed with 10% Neutral-buffered Formalin (NBF). After 24 hrs post fixation in 10% NBF, hearts were embedded in paraffin and 5 um sections were mounted onto treated slides, and stained with hematoxylin and Eosin (H&E) and co-stained with Masson's trichrome. Tissue fibrosis was quantified by counting blue stained interstitial collagen within three random fields using Image J and averaged.

Metabolomic Analysis

We used Biocrates AbsoluteIDQ p180 kit to analyze cardiac metabolites with UPLC-MS/MS Xevo TQ, Waters, Pittsburgh, Pa., USA) in the Einstein Stable Isotope and Metabolomics Core, according to the manufacturer's instructions (BIOCRATES Life Sciences AG, Innsbruck, Austria). Heart tissue samples were weighed, homogenized with 8 times of 2.5 mM ammonium acetate in methanol, and 20 uL of the extraction from each sample was used for the assay. A pooled quality control (QC) sample was added to the sample list. This QC sample was plated at different positions on the 96-well plate and injected multiple times to calculate the coefficient of variation (CV) for data quality control. The data set was imported into SIMCA-p software for multivariate analysis, and unsupervised principle component analysis (PCA) and partial least squares-discriminant analysis (PLS-DA) model were established.

Doxorubicin Challenge

Beginning at 15 mo of age, male and female C57BL/6 mice were preemptively treated with vehicle or L2-Cmu for 3 mo. Mice were then subsequently evaluated for baseline cardiac function by echocardiogram at 18 mo of age, prior to a low-dose, DOX challenge (4 mg/kg/wk; i.p) on consecutive weeks in females (four doses) and males (three doses), respectively. Animals remained on vehicle or mAb treatment throughout the chemotherapeutic challenge and were re-assessed for effects on cardiac function one week following the final dose. In addition, a ‘clinical’ frailty index was determined for all mice following DOX treatment, as described⁴⁵.

Histopathology

Complete histopathology was performed in 24 mo old male and female mice following 6 mo of mAb treatment, as well as in female mice at death from the longevity study. In brief, a gross evaluation was conducted when possible and then a complete necropsy was performed. Tissues were infiltrated with paraffin and H&E sections were obtained. Slides were shipped to the University of Texas at San Antonio Pathology Core and evaluated by two pathologists who were blinded to the experimental groups. Diagnosis of each histopathological change was made using histological classifications for aging mice as previously described^{29,45,46}. In brief, a list of lesions was compiled for each mouse that included both neoplastic and non-neoplastic diseases. Based on these histopathological data, tumor burden, disease burden, and severity of each lesion in each mouse were assessed. The tumor burden was calculated as the sum of the different types of tumors in each mouse. The disease burden was similarly calculated as the sum of the histopathological changes in a mouse and severity of neoplastic and renal lesions was assessed using an established grading system. The probable cause of death was determined independently by both pathologists based on the severity of the pathology found at necropsy. In cases with neoplastic lesions, mice with Grade 3 or 4 lesions were categorized as death by neoplastic disease. In more than 90% of the cases, there was agreement by the two pathologists. In cases where the two pathologists did not agree or where disease did not appear severe enough, the cause of death was categorized as unknown.

Blood Measures

Clinical blood chemistries and related measures were determined in whole blood and serum by Antech Diagnostics (New York, N.Y.). Basal insulin was measured by a rat/mouse ELISA (EMD Millipore, Inc) with rat insulin standards and plasma IGF-1 levels were measured using the Mouse/Rat IGF-1 Quantikine ELISA Kit (MG100; R&D Systems). In addition, a Bio-Plex MAGPIX Multiplex Reader (Biorad Inc., Hercules, Calif.) was used to measure 25 inflammatory mediators simultaneously in plasma, including: G-CSF, GM-CSF, IFN-γ, IL-1α, IL-1β, IL-2, IL-4, IL-5, IL-6, IL-7, IL-9, IL-10, IL-12 (p40), IL-12 (p70), IL-13, IL-15, IL-17, CXCL-10, CXCL-1, MCP-1, MIP-1α, MIP-1β, MIP-2, RANTES, and TNF-α (MCYTOMAG-70K-PMX; EMD Millipore, Billerica, Mass.).

RNA Isolation and Expression

Total RNA from frozen tissues were isolated using Trizol. First-strand complementary DNA (cDNA) was synthesized with random primers and total RNA as a template using Biorad iScript cDNA Synthesis Kit. All qPCR reactions were carried out using Biorad Sso Advanced SYBR Green mix on a Biorad CFX384 qRT-PCR Machine. Expression of p16 in tissues was determined using previously reported primers⁴⁸, and normalized to β-actin expression.

Protein Isolation and Western Blot

For standard western blotting, RIPA buffer and protein content was determined using the BCA protein assay (Sigma, St. Louis, Mo.) with BSA as a standard. Western blotting was performed similar as described^{43,48}. Protein was separated on Bis Tris Stain-Free gels (4-20%) and electrophoresed at 120V constant for 90 min (n=8 per group). Prior to transfer, stain-free gels were imaged on a Biorad Chemidoc MP Imaging System (Biorad, Hercules, Calif.) to confirm equal protein load, and were then wet transferred onto PVDF membranes at 100V constant for 1 hr and equal transfer was routinely confirmed by Ponceau S stain. Following block in 5% milk, membranes were incubated with an appropriate primary antibody from Cell Signaling (Danvers, Mass.) against pAkt^{Ser473} (#4060), total Akt (#4691), p-p44/42MAPK^{Thr202/Tyr204} (#9101) total p44/42 MAPK (#4695), pS6 (#5364), Total S6 (#2217), total IGF-1R (#9750) InsRβ (#3025), pNFκB (#3033) and Total NFκB (#8242) overnight at 4° C. Following a 1 hr incubation with the appropriate secondary antibody, Clarity Western ECL Substrate (Biorad) was applied to the membrane and bands were visualized using a Biorad Chemidoc MP to first pixel saturation and densitometry performed using Image Lab (Biorad, Hercules, Calif.).

Immunoprecipitation

For immunoprecipitation assays, NIH-3T3 cell protein was extracted with a non-denaturing cell extraction buffer (Invitrogen/ThermoFisher, Carlsbad, Calif.). Immunoprecipitation was then performed using the Catch and Release Immunoprecipitation Kit (EMD Millipore), according to the manufacturer's instructions with 250 ug of total protein and 1 ug of an anti-IGF-1R antibody (#9750, Cell Signaling). Following electrophoresis and transfer, membranes were blotted with either a pTyr antibody (E0614, Cell Signaling) for IGF-1R activation, or an anti-IGF-1R antibody for total levels. For IGF-1R/InsR HybridR activation, IGF-1R immunoprecipitates were probed with an antibody against the InsR specific pTyr¹³³⁴ residue (#44809G, Invitrogen/ThermoFisher), similar as described⁴⁹, and total HybridR determined by immunoblotting with an Anti-InsRβ antibody (#3025).

Statistics

All values are presented as means±SE. Longitudinal measures were assessed by repeated-measures ANOVA (using age as a fixed effect and subject ID as a random effect) and cross sectional data were assessed by one-way ANOVA. When a significant effect was observed, planned contrasts (Turkey Honest Significant Difference [HSD] method) were applied to determine individual differences between groups. Non-parametric tests were analyzed by the Kruskal-Wallis procedure and post hoc comparisons conducted with the Mann-Whitney U test when appropriate. The total frequency and grade of pathologic lesions were compared between genotypes using a chi-square test. When the expected frequencies were too small for the chi-square test, the data were analyzed using the Fisher's exact test. For survival analysis, as animals were obtained in 6 separate batches due to operational limitations and grouped in cages, the survival curves were plotted using the Kaplan-Meier method and the treatment effect was evaluated using Cox proportional hazard regression with cage assignment included as a random effect and batch as a covariate, using statistical software R (version 3.4.1) with library “coxme”. Effects on maximum lifespan were determined by setting the threshold for lifespan to the 90th percentile for both groups combined, as described⁵⁰. All other statistical analyses were performed using either SPSS (SPSS Inc, Chicago, Ill.) or JMP software version 9 (SAS Institute Inc., Cary, N.C.). A P≤0.05 was considered statistically significant.

### Example 2

The following example demonstrates a method of administering doses of an IGF-1R inhibitor to human subjects and testing the therapeutic efficacy thereof.

Post-menopausal female patients are randomly assigned to one of four treatment groups: low dose, medium dose, high dose, or placebo. In some variations of the experiment, patients are determined to be cancer-free based on evaluation prior to enrollment. In some variations, patients are excluded if the patients have past cancer diagnoses (as determined from medical record or self-reporting).

Prior to any treatment, baseline measurements of IGF-1, growth hormone (GH), growth hormone releasing hormone (GHRH) and inflammatory markers are measured according to methods known in the art. The GHRH+arginine test is carried out as essentially described in Glynn and Agha (2012), supra. Briefly, arginine is administered to the subject by intravenous infusion (0.5 g/kg body weight) with an intravenous bolus of GHRH (1 mcg/kg body weight). Serum samples are obtained every 15-30 min for two hours. Glucagon stimulation tests and insulin tolerance tests are also performed prior to treatment. In the latter, a bolus of intravenous insulin (0.15 units/kg) is administered to the subject and GH levels are measured every 15-30 min for two hours. For the glucagon stimulation test, glucagon (1.0-1.5 mg) is administered intramuscularly and serum samples are taken for GH between 90 and 240 min post injection. Inflammatory markers TNF-α, CRP, IL-6, IL-4, IL-5, CXCL-1, 1I-12p40, MIP-1α, and MIP-2 are measured from patient serum samples.

Patients are measured for baseline body weight, blood pressure, and body fat. Cardiac health parameters, including, isovolumic LV relaxation time (IVRT), ratio of peak early (E) to peak atrial (A) Doppler mitral valve flow velocity, deceleration time (DT) of early Doppler mitral valve flow velocity, and ratio of pulmonary vein systolic (S) and diastolic (D) flow velocities are measured by blood flow Doppler assessment.

The low dose group is intravenously administered 3 mg/kg dose of the IGF-1R inhibitor, while patients in the medium dose group and high dose group are intravenously administered 6 mg/kg and 12 mg/kg, respectively. The placebo group is intravenously administered a saline solution. Each group is administered the assigned dose once every two weeks for 8 weeks. In some aspects, the IGF-1R is an antigen-binding protein as described herein. In exemplary aspects, the IGF-1R is ganitumab or a similar antibody, e.g., L2-C.

Serum is collected 3 days post-treatment and 1, 2, 4, 6, 8, 10, and 12 weeks after the first treatment. The tests performed to obtain baseline measurements are repeated to obtain post-treatment measurements at these time points. Adverse events are recorded during the treatment and post-treatment periods.

In exemplary aspects, the clinical trial is carried beyond the 12 week period. An extension phase is carried out with the same patients and the patients remain in the same assigned groups. Serum is collected at additional time points (at 16, 20, and 24 weeks after the first treatment). Measurements taken at baseline are repeated at these additional time points. At 24 weeks, treatment begins again. The low dose group is intravenously administered 3 mg/kg dose of the IGF-1R inhibitor, while patients in the medium dose group and high dose group are intravenously administered 6 mg/kg and 12 mg/kg, respectively. The placebo group is intravenously administered a saline solution. Each group is administered the assigned dose once every two weeks for 8 weeks. Serum and baseline measurements are collected every two weeks following the second 8-week treatment period for the subsequent four months.

### REFERENCES CITED HEREIN

1. Kenyon et al., Nature 366, 461-464 (1993).
2. Fabrizio et al., Science 292, 288-290 (2001).
3. Clancy et al., Science 292, 104-106 (2001).
4. Bartke et al., Growth Horm IGF Res 27, 41-45 (2016).
5. Barzilai et al., Diabetes 61, 1315-1322 (2012).
6. Holzenberger et al., Nature 421, 182-187 (2003).
7. Bokov et al., PLoS One 6, e26891 (2011).
8. Xu et al., Aging Cell 13, 19-28 (2014).
9. Brown-Borg, Am J Physiol Endocrinol Metab 309, E503-510 (2015).
10. Longo, et al., Aging Cell 14, 497-510 (2015).
11. Junnila et al., Nat Rev Endocrinol 9, 366-376 (2013).
12. Milman et al., Cell Metab 23, 980-989 (2016).
13. Suh et al., Proc Natl Acad Sci USA 105, 3438-3442 (2008).
14. Tazearslan et al., Aging Cell 10, 551-554 (2011).
15. Guevara-Aguirre et al., Sci Transl Med 3, 70ra13 (2011).
16. Milman et al., Aging Cell 13, 769-771 (2014).
17. Panici et al., FASEB J 24, 5073-5079 (2010).
18. Tolcher et al., J Clin Oncol 27, 5800-5807 (2009).
19. Beltran et al., Mol Cancer Ther 8, 1095-1105 (2009).
20. Tap et al., J Clin Oncol 30, 1849-1856 (2012).
21. Calzone et al., PLoS One 8, e55135 (2013).
22. Moody et al., J Endocrinol 221, 145-155 (2014).
23. Ungvari, Z. & Csiszar, A., J Gerontol A Biol Sci Med Sci 67, 599-610 (2012).
24. Moellendorf, S., et al. Am J Physiol Endocrinol Metab 303, E213-222 (2012).
25. Quarles, E. K., et al. Ageing Res Rev 23, 101-115 (2015).
26. Ock, S., et al., Endocrinology 157, 336-345 (2016).
27. Harrison, D. E., et al., Nature 460, 392-395 (2009).
28. Chiao, Y. A., et al., Aging (Albany N.Y.) 8, 314-327 (2016).
29. Ikeno, Y., et al. J Gerontol A Biol Sci Med Sci 64, 522-529 (2009).
30. Ma et al., Br J Clin Pharmacol 77, 917-928 (2014).
31. Barzilai et al., Cell Metab 23, 1060-1065 (2016).
32. Strong et al., Aging Cell 15, 872-884 (2016).
33. Harrison, D. E., et al., Aging Cell 13, 273-282 (2014).
34. Austad, S. N. & Fischer, K. E., Cell Metab 23, 1022-1033 (2016).
35. Spadaro, O., et al., Cell Rep 19, 225-234 (2017).
36. Ni, F., et al. Nat Commun 4, 1479 (2013).
37. Bilbao et al., EMBO Mol Med 6, 1423-1435 (2014).
38. Gong, Z., et al. Aging Cell 13, 408-418 (2014).
39. Steger, R. W., Bartke, A. & Cecim, M. J Reprod Fertil Suppl 46, 61-75 (1993).
40. Bitto, A., et al. Elife 5 (2016).
41. Huffman, D. M., et al. Cancer Prev Res (Phila) 6, 177-187 (2013).
42. Huffman, D. M., et al. Cancer Res 67, 417-424 (2007).
43. Huffman, D. M., et al. Aging Cell 15, 181-186 (2016).
44. Lutz, S. E., et al., J Neurosci 29, 7743-7752 (2009).
45. Ikeno, Y., et al. J Gerontol A Biol Sci Med Sci 60, 1510-1517 (2005).
46. Ikeno et al., J Gerontol A Biol Sci Med Sci 58, 291-296 (2003).
47. Baker, D. J., et al. Nature 530, 184-189 (2016).
48. Huffman, D. M., et al. Am J Physiol Regul Integr Comp Physiol 294, R1618-1627 (2008).
49. Slaaby, Sci Rep 5, 7911 (2015).
50. Gao et al., BMC Med Res Methodol 8, 49 (2008).

All references, including publications, patent applications, and patents, cited herein are hereby incorporated by reference to the same extent as if each reference were individually and specifically indicated to be incorporated by reference and were set forth in its entirety herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of improvement, preservation, prophylaxis, or inhibition-of-deterioration of a healthspan parameter of a mammalian subject, the method comprising administering to the subject a composition that comprises an insulin-like growth factor-1 receptor (IGF-1 R) inhibitor,
wherein the composition is administered in an amount effective to improve, provide prophylaxis for, or inhibit-the-deterioration of the healthspan parameter, and
wherein the healthspan parameter is a cardiac health or function, a motor function, a cognitive function, body fatness/leanness, muscle strength, exercise endurance, freedom from malignancy, or an inflammation.

2-6. (canceled)

7. The method of claim 1, wherein the healthspan parameter comprises a motor function, and the method, further comprises a step, prior to the administering step, of screening a motor function of the subject and identifying a motor function deficit, compared to a motor function index, or motor function deterioration, compared to a measurement of the motor function from prior screening of the subject.

8. The method of claim 1, wherein the healthspan parameter comprises a motor function, and the method, further comprises administering to the subject a myostatin inhibitor.

9-12. (canceled)

13. The method of claim 1, wherein the healthspan parameter comprises a cardiac health or function, and the method further comprises a step, prior to the administering step, of screening a cardiac function of the subject or in a sample from the subject, and identifying a cardiac function deficit, compared to a cardiac function index, or identifying a cardiac function deterioration, compared to a measurement of said cardiac function from prior screening of the subject.

14. (canceled)

15. The method of claim 1, wherein the healthspan parameter comprises a cardiac health or function, and the method further comprises administering to the subject a statin, a beta blocker, or an inotropic agent.

16-19. (canceled)

20. The method of claim 1, wherein the healthspan parameter comprises an inflammation, and the method further comprises, further comprising a step, prior to the administering step, of screening a sample from the subject and identifying an elevated inflammatory marker, compared to an index for said marker, or identifying an increase in said inflammatory marker, compared to a measurement from prior screening of the subject.

21. The method of claim 1, wherein the healthspan parameter comprises an inflammation, and the method further comprises administering to the subject a cyclooxygenase inhibitor, a platelet aggregation inhibitor, a statin, a beta-adrenoreceptor antagonist, or an angiotensin converting enzyme (ACE) inhibitor.

22-25. (canceled)

26. The method of claim 21, wherein the healthspan parameter comprises body fatness/leanness, and the method further comprises administering to the subject an appetite suppressant.

27. The method of claim 1, wherein the subject is an adult free of diagnosed or self-reported malignancy (cancer).

28-32. (canceled)

33. The method of claim 1, wherein the subject is a human female.

34. The method of claim 33, wherein the subject is a menopausal or post-menopausal female.

35. (canceled)

36. The method of claim 34, wherein the subject is at least 30 years old.

37. The method of claim 34, wherein the subject has experienced aged-related deterioration of growth hormone.

38. The method of claim 34, wherein the subject is experiencing or has experienced somatopause.

39. The method of claim 3, comprising a step, prior to the administering step, of diagnosing somatopause through hormone pulsatility measurement.

40. The method of claim 1, wherein IGF-1 R inhibitor comprises an antigen binding protein that binds to an epitope of IGF-1 or IGF-1 R and inhibits IGF-1 binding to IGF-1 R.

41. The method of claim 40, wherein the IGF-1 R inhibitor comprises an antibody, or comprises an antigen binding fragment of said antibody.

42. The method of claim 41, wherein the IGF-1 R inhibitor binds to an epitope of IGF-1 R.

43. The method of claim 42, wherein the IGF-1 R inhibitor binds to an epitope within the L2 domain of the alpha subunit of human IGF-1 R.

44. The method of claim 42, wherein the IGF-1R inhibitor comprises an antibody.

45. The method of claim 44, wherein the antibody comprises a light chain (LC) CDR1 comprising the amino acid sequence of SEQ ID NO: 1 or an amino acid sequence comprising at least 75% sequence identity to SEQ ID NO: 1; a LC CDR2 comprising the amino acid sequence of SEQ ID NO: 2 or an amino acid sequence comprising at least 75% sequence identity to SEQ ID NO: 2; and a LC CDR3 comprising the amino acid sequence of SEQ ID NO: 3 or an amino acid sequence comprising at least 75% sequence identity to SEQ ID NO: 3.

46. The method of claim 44, wherein the antibody comprises a heavy chain (HC) CDR1 comprising the amino acid sequence of SEQ ID NO: 4 or an amino acid sequence comprising at least 75% sequence identity to SEQ ID NO: 4; a HC CDR2 comprising the amino acid sequence of SEQ ID NO: 5 or an amino acid sequence comprising at least 75% sequence identity to SEQ ID NO: 5; and a HC CDR3 comprising the amino acid sequence of SEQ ID NO: 6 or an amino acid sequence comprising at least 75% sequence identity to SEQ ID NO: 6.

47. The method of claim 44, wherein the antibody comprises a LC that comprises the amino acid sequence of SEQ ID NO: 7, or an amino acid sequence comprising at least 75% sequence identity to SEQ ID NO: 7.

48. The method of claim 44, wherein the antibody comprises a HC that comprises the amino acid sequence of SEQ ID NO: 8, or an amino acid sequence comprising at least 75% sequence identity to SEQ ID NO: 8.

49. The method of claim 44, wherein the antibody comprises a light chain (LC) CDR1 comprising the amino acid sequence of SEQ ID NO: 322 or an amino acid sequence comprising at least 75% sequence identity to SEQ ID NO: 322; a LC CDR2 comprising the amino acid sequence of SEQ ID NO: 323 or an amino acid sequence comprising at least 75% sequence identity to SEQ ID NO: 323; and a LC CDR3 comprising the amino acid sequence of SEQ ID NO: 324 or an amino acid sequence comprising at least 75% sequence identity to SEQ ID NO: 324.

50. The method of claim 44, wherein the antibody comprises a heavy chain (HC) CDR1 comprising the amino acid sequence of SEQ ID NO: 325 or an amino acid sequence comprising at least 75% sequence identity to SEQ ID NO: 325; a HC CDR2 comprising the amino acid sequence of SEQ ID NO: 326 or an amino acid sequence comprising at least 75% sequence identity to SEQ ID NO: 326; and a HC CDR3 comprising the amino acid sequence of SEQ ID NO: 327 or an amino acid sequence comprising at least 75% sequence identity to SEQ ID NO: 327.

51. The method of claim 44, wherein the antibody comprises a LC that comprises the amino acid sequence of SEQ ID NO: 304, or an amino acid sequence comprising at least 75% sequence identity to SEQ ID NO: 304.

52. The method of claim 44, wherein the antibody comprises a HC that comprises the amino acid sequence of SEQ ID NO: 305, or an amino acid sequence comprising at least 75% sequence identity to SEQ ID NO: 305.

53-74. (canceled)

75. The method of claim 44, further comprising administering to the subject an m-Tor inhibitor or metformin.

76. (canceled)

77. The method of claim 44, wherein the subject is female, and wherein the method further comprises administering an estrogen replacement therapy to the subject.


SEQUENCE LISTING (327 sequences)

SEQ ID NO: 1 - protein, 16 aa, Artificial Sequence
  Synthetic Polypeptide
  MISC_FEATURE: Light Chain CDR1
  MISC_FEATURE: Aa 24-39 of SEQ 7
RSSQSLLHSNGYNYLD

SEQ ID NO: 2 - protein, 7 aa, Artificial Sequence
  Synthetic Peptide
  MISC_FEATURE: Light Chain CDR2
  MISC_FEATURE: Aa 55-61 of SEQ 7
LGSNRAS

SEQ ID NO: 3 - protein, 9 aa, Artificial Sequence
  Synthetic Peptide
  MISC_FEATURE: Light Chain CDR 3
  MISC_FEATURE: Aa 94-102 of SEQ 7
MQGTHWPLT

SEQ ID NO: 4 - protein, 6 aa, Artificial Sequence
  Synthetic Peptide
  MISC_FEATURE: Heavy Chain CDR1
  MISC_FEATURE: Aa 31-36 of SEQ 8
SSNWWS

SEQ ID NO: 5 - protein, 16 aa, Artificial Sequence
  Synthetic Peptide
  MISC_FEATURE: Heavy Chain CDR2
  MISC_FEATURE: Aa 51-66 of SEQ 8
EIYHSGSTNYNPSLKS

SEQ ID NO: 6 - protein, 10 aa, Artificial Sequence
  Synthetic Peptide
  MISC_FEATURE: Heavy Chain CDR3
  MISC_FEATURE: Aa 99-108 of SEQ 8
WTGRTDAFDI

SEQ ID NO: 7 - protein, 112 aa, Artificial Sequence [sequence omitted]

SEQ ID NO: 8 - protein, 119 aa, Artificial Sequence [sequence omitted]

SEQ ID NO: 9 - protein, 107 aa, Artificial Sequence [sequence omitted]

SEQ ID NO: 10 - protein, 330 aa, Artificial Sequence [sequence omitted]

SEQ ID NO: 11 - protein, 706 aa, Artificial Sequence
  Synthetic Peptide
  MISC_FEATURE: IGF-1R
EICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPE

SEQ ID NO: 12 - protein, 163 aa, Artificial Sequence
  Synthetic Peptide
  MISC_FEATURE: L2
NLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLI

SEQ ID NO: 13 - protein, 160 aa, Artificial Sequence
  Synthetic Peptide
  MISC_FEATURE: L2
KVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCE

SEQ ID NO: 14 - protein, 163 aa, Artificial Sequence
  Synthetic Peptide
  MISC_FEATURE: L2
CPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCES

SEQ ID NO: 15 - protein, 16 aa, Artificial Sequence
  Synthetic Peptide
  MISC_FEATURE: Light chain CDR1
  MISC_FEATURE (1)..(16)
RSSQSLLHSNGYNYLD

SEQ ID NO: 16 - protein, 11 aa, Artificial Sequence
  Synthetic Peptide
  MISC_FEATURE: Light chain CDR1
  MISC_FEATURE (5)..(5): Xaa is glycine or serine
  MISC_FEATURE (6)..(6): Xaa is isoleucine or valine
  MISC_FEATURE (7)..(7): Xaa is glycine or serine
  MISC_FEATURE (8)..(8): Xaa is any amino acid
  MISC_FEATURE (9)..(9): Xaa is tyrosine or phenyalanine
  MISC_FEATURE (11)..(11): Xaa is alanine or asparagine
RASQXXXXXLX

SEQ ID NO: 17 - protein, 11 aa, Artificial Sequence
  Synthetic Peptide
  MISC_FEATURE: Light chain CDR1
  MISC_FEATURE (6)..(6): Xaa is leucine or serine
  MISC_FEATURE (7)..(11): Xaa is independently any amino acid
RSSQSXXXXXX

SEQ ID NO: 18 - protein, 16 aa, Artificial Sequence
  Synthetic Peptide
  MISC_FEATURE: light chain CDR1
  MISC_FEATURE (9)..(9): Xaa is serine or threonine residue
  MISC_FEATURE (10)..(10): Xaa is asparagine, serine or histidine residue
  MISC_FEATURE (14)..(14): Xaa is tyrosine or phenylalanine residue
  MISC_FEATURE (16)..(16): Xaa is aspartate or asparagine residue
RSSQSLLHXXGYNXLX

SEQ ID NO: 19 - protein, 13 aa, Artificial Sequence
  Synthetic Peptide
  MISC_FEATURE: light chain CDR1
  MISC_FEATURE (6)..(6): Xaa is serine or aspartate residue
  MISC_FEATURE (8)..(8): Xaa is alanine or aspartate residue
  MISC_FEATURE (9)..(9): Xaa is serine or asparagine residue
TRSSGXIXXNYVQ

SEQ ID NO: 20 - protein, 11 aa, Artificial Sequence
  Synthetic Polypeptide
  MISC_FEATURE: light chain CDR1
  MISC_FEATURE (5)..(5): Xaa is glycine or serine residue
  MISC_FEATURE (6)..(6): Xaa is isoleucine, valine or proline residue
  MISC_FEATURE (7)..(7): Xaa is serine, glycine or tyrosine residue
  MISC_FEATURE (8)..(8): Xaa is any amino acid
  MISC_FEATURE (9)..(9): Xaa is phenylalanine, tyrosine, asparagine or tryptophan residue
  MISC_FEATURE (11)..(11): Xaa is alanine or asparagine residue
RASQXXXXXLX

SEQ ID NO: 21 - protein, 7 aa, Artificial Sequence
  Synthetic Polypeptide
  MISC_FEATURE: Light chain CDR2
LGSNRAS

SEQ ID NO: 22 - protein, 7 aa, Artificial Sequence
  Synthetic Polypeptide
  MISC_FEATURE: Light chain CDR2
AASTLQS

SEQ ID NO: 23 - protein, 7 aa, Artificial Sequence
  Synthetic Polypeptide
  MISC_FEATURE: Light chain CDR2
  MISC_FEATURE (4)..(4): Xaa can be any naturally occurring amino acid
EDNXRPS

SEQ ID NO: 24 - protein, 7 aa, Artificial Sequence
  Synthetic Polypeptide
  MISC_FEATURE: Light Chain CDR2
  MISC_FEATURE (2)..(2): Xaa is glycine or valine residue
  MISC_FEATURE (3)..(3): Xaa is serine or phenylalanine residue
  MISC_FEATURE (4)..(4): Xaa is asparagine, tyrosine or threonine residue
  MISC_FEATURE (6)..(6): Xaa is alanine or aspartate residue
LXXXRXS

SEQ ID NO: 25 - protein, 7 aa, Artificial Sequence
  Synthetic Polypeptide
  MISC_FEATURE: light chain CDR2
  MISC_FEATURE (2)..(2): Xaa is alanine or threonine residue
  MISC_FEATURE (4)..(4): Xaa is threonine or glycine residue
  MISC_FEATURE (6)..(6): Xaa is glutamine or glutamate residue
AXSXLXS

SEQ ID NO: 26 - protein, 7 aa, Artificial Sequence
  light chain CDR2
  MISC_FEATURE (1)..(1): Xaa is glutamate, glutamine or glycine residue
  MISC_FEATURE (2)..(2): Xaa is aspartate or lysine residue
  MISC_FEATURE (4)..(4): Xaa is any amino acid residue
XXNXRPS

SEQ ID NO: 27 - protein, 9 aa, Artificial Sequence
  Synthetic Polypeptide
  MISC_FEATURE: Light chain CDR3
  MISC_FEATURE (8)..(8): Xaa can be any naturally occuring amino acid
MQALQTPXT

SEQ ID NO: 28 - protein, 9 aa, Artificial Sequence
  Synthetic Polypeptide
  MISC_FEATURE: Light chain CDR3
  MISC_FEATURE (3)..(3): Xaa is arginine or serine
  MISC_FEATURE (4)..(4): Xaa is asparagine or serine
  MISC_FEATURE (5)..(5): Xaa is serine or asparagine
  MISC_FEATURE (6)..(6): Xaa is glycine, alanine, valine, leucine, isoleucine, proline, phenylalinine, methionine, tryptophan or cysteine
QQXXXXPLT

SEQ ID NO: 29 - protein, 10 aa, Artificial Sequence
  Synthetic Polypeptide
  MISC_FEATURE: Light chain CDR3
  MISC_FEATURE (8)..(9): Xaa is arginine, valine, or isoleucine or no amino acid
QSYDSSNXXV

SEQ ID NO: 30 - protein, 9 aa, Artificial Sequence
  Synthetic Polypeptide
  MISC_FEATURE: light chain CDR3
  MISC_FEATURE (2)..(2): Xaa is glutamine or glutamate residue
  MISC_FEATURE (3)..(3): Xaa is alanine, glycine, serine or threonine residue
  MISC_FEATURE (4)..(4): Xaa is leucine or threonine residue
  MISC_FEATURE (5)..(5): Xaa is glutamine, glutamate or histidine residue
  MISC_FEATURE (6)..(6): Xaa is threonine, tryptophan, methionine or valine residue
  MISC_FEATURE (8)..(8): Xaa is an amino acid with a nonpolar side chain
  MISC_FEATURE (9)..(9): Xaa is threonine, serine or alanine residue
MXXXXXPXX

SEQ ID NO: 31 - protein, 9 aa, Artificial Sequence
  Synthetic Polypeptide
  MISC_FEATURE: light chain CDR3
  MISC_FEATURE (3)..(3): Xaa is arginine, serine, leucine or alanine residue
  MISC_FEATURE (4)..(4): Xaa is asparagine, serine or histidine residue
  MISC_FEATURE (5)..(5): Xaa is serine or asparagine residue
  MISC_FEATURE (6)..(6): Xaa is an amino acid with a nonpolar side chain
  MISC_FEATURE (8)..(8): Xaa is leucine, isoleucine, tyrosine or tryptophan residue
QQXXXXPXT

SEQ ID NO: 32 - protein, 10 aa, Artificial Sequence
  Synthetic Polypeptide
  MISC_FEATURE: light chain CDR3
  MISC_FEATURE (4)..(4): Xaa is aspartate or glutamine residue
  MISC_FEATURE (6)..(6): Xaa is serine or aspartate residue
  MISC_FEATURE (8)..(8): Xaa is glutamine, valine or tryptophan residue
  MISC_FEATURE (9)..(9): Xaa is arginine residue or no residue
QSYXSXNXXV

SEQ ID NO: 33 - protein, 6 aa, Artificial Sequence
  Synthetic Polypeptide
  MISC_FEATURE: Heavy chain CDR1
SSNWWS

SEQ ID NO: 34 - protein, 5 aa, Artificial Sequence
  Synthetic Polypeptide
  MISC_FEATURE: Heavy chain CDR1
  MISC_FEATURE (1)..(1): Xaa can be any naturally occurring amino acid
XYYWS

SEQ ID NO: 35 - protein, 5 aa, Artificial Sequence
  Synthetic Polypeptide
  MISC_FEATURE: Heavy chain CDR1
  MISC_FEATURE (5)..(5): Xaa is serine or histidine
SYAMX

SEQ ID NO: 36 - protein, 6 aa, Artificial Sequence
  Synthetic Polypeptide
  MISC_FEATURE: heavy chain CDR1
  MISC_FEATURE (1)..(1): Xaa is serine residue or no residue
  MISC_FEATURE (2)..(2): Xaa is serine or asparagine residue
  MISC_FEATURE (3)..(3): Xaa is asparagine and isoleucine residue
XXXWWS

SEQ ID NO: 37 - protein, 5 aa, Artificial Sequence
  Synthetic Polypeptide
  MISC_FEATURE: heavy chain CDR1
  MISC_FEATURE (1)..(1): Xaa is glycine, asparagine or aspartate residue
  MISC_FEATURE (2)..(2): Xaa is tyrosine or phenylalanine residue
XXYWS

SEQ ID NO: 38 - protein, 5 aa, Artificial Sequence
  Synthetic Polypeptide
  MISC_FEATURE: heavy chain CDR1
  MISC_FEATURE (3)..(3): Xaa is alanine or glycine residue
  MISC_FEATURE (4)..(4): Xaa is methionine or isoleucine residue
  MISC_FEATURE (5)..(5): Xaa is serine or histidine residue
SYXXX

SEQ ID NO: 39 - protein, 16 aa, Artificial Sequence
  Synthetic Polypeptide
  MISC_FEATURE: Heavy chain CDR2
  MISC_FEATURE (1)..(1): Xaa = glutamic acid or isoleucine
  MISC_FEATURE (2)..(2): Xaa = isoleucine or valine
  MISC_FEATURE (3)..(3): Xaa = tyrosine or asparagine
  MISC_FEATURE (4)..(4): Xaa = histidine or tyrosine
  MISC_FEATURE (9)..(9): Xaa = asparagine or tyrosine
XXXXSGSTXYNPSLKS

SEQ ID NO: 40 - protein, 17 aa, Artificial Sequence
  Synthetic Polypeptide
  MISC_FEATURE: Heavy chain CDR2
  MISC_FEATURE (1)..(1): Xaa = any amino acid
  MISC_FEATURE (4)..(4): Xaa = glycine or serine
  MISC_FEATURE (7)..(7): Xaa = glycine or serine
XISXSGXSTYYADSVKG

SEQ ID NO: 41 - protein, 16 aa, Artificial Sequence
  Synthetic Polypeptide
  MISC_FEATURE: heavy chain CDR2
  MISC_FEATURE (1)..(1): Xaa is glutamate, tyrosine or serine residue
  MISC_FEATURE (2)..(2): Xaa is isoleucine or valine residue
  MISC_FEATURE (3)..(3): Xaa is tyrosine, asparagine or serine residue
  MISC_FEATURE (4)..(4): Xaa is histidine, tyrosine, aspartate,or proline residue
  MISC_FEATURE (5)..(5): Xaa is serine or arginine residue
  MISC_FEATURE (7)..(7): Xaa is serine or arginine residue
  MISC_FEATURE (9)..(9): Xaa is asparagine or tyrosine residue
  MISC_FEATURE (15)..(15): Xaa is lysine or glutamate residue
XXXXXGXTXYNPSLXS

SEQ ID NO: 42 - protein, 17 aa, Artificial Sequence
  Synthetic Polypeptide
  MISC_FEATURE: heavy chain CDR2
  MISC_FEATURE (1)..(1): Xaa is threonine, alanine, valine or tyrosine residue
  MISC_FEATURE (4)..(4): Xaa is glycine, serine or tyrosine residue
  MISC_FEATURE (5)..(5): Xaa is serine, asparagine or aspartate residue
  MISC_FEATURE (6)..(6): Xaa is glycine or serine residue
  MISC_FEATURE (7)..(7): Xaa is glycine, serine or aspartate residue
  MISC_FEATURE (8)..(8): Xaa is serine, threonine or asparagine residue
  MISC_FEATURE (9)..(9): Xaa is threonine, lysine or isoleucine residue
XISXXXXXXYYADSVKG

SEQ ID NO: 43 - protein, 8 aa, Artificial Sequence
  Synthetic Polypeptide
  MISC_FEATURE: Heavy chain CDR3
SRLDAFDI

SEQ ID NO: 44 - protein, 10 aa, Artificial Sequence
  Synthetic Polypeptide
  MISC_FEATURE: Heavy chain CDR3
  MISC_FEATURE (2)..(2): Xaa can be any naturally occurring amino acid
SXYDYYGMDV

SEQ ID NO: 45 - protein, 11 aa, Artificial Sequence
  Synthetic Polypeptide
  MISC_FEATURE: Heavy chain CDR3
  MISC_FEATURE (3)..(3): Xaa can be any naturally occurring amino acid
  MISC_FEATURE (5)..(5): Xaa can be any naturally occurring amino acid
HRXDXAWYFDL

SEQ ID NO: 46 - protein, 4 aa, Artificial Sequence
  Synthetic Polypeptide
  MISC_FEATURE: Heavy chain CDR3
DSSG

SEQ ID NO: 47 - protein, 6 aa, Artificial Sequence
  Synthetic Polypeptide
  MISC_FEATURE: Heavy chain CDR1
SSNWWS

SEQ ID NO: 48 - protein, 5 aa, Artificial Sequence
  Synthetic Polypeptide
  MISC_FEATURE: Heavy chain CDR1
  MISC_FEATURE (1)..(1): Xaa can be any naturally occurring amino acid
XYYWS

SEQ ID NO: 49 - protein, 5 aa, Artificial Sequence
  Synthetic Polypeptide
  MISC_FEATURE: Heavy chain CDR1
  MISC_FEATURE (5)..(5): Xaa is serine or histidine
SYAMX

SEQ ID NO: 50 - protein, 12 aa, Artificial Sequence
  Synthetic Polypeptide
  MISC_FEATURE: heavy chain CDR3
  MISC_FEATURE (1)..(1): Xaa is glutamate or no residue
  MISC_FEATURE (2)..(2): Xaa is tyrosine, glycine or serine or no residue
  MISC_FEATURE (3)..(3): Xaa is serine, asparagine, tryptophan or glutamate or no residue
  MISC_FEATURE (4)..(4): Xaa is serine, aspartate, tryptophan, alanine, arginine, threonine, glutamine, leucine or glutamate or no residue
  MISC_FEATURE (5)..(5): Xaa is serine, glycine, asparagine, threonine, tryptophan, alanine, valine or isoleucine residue
  MISC_FEATURE (6)..(6): Xaa is arginine, glutamine, tyrosine, valine, alanine, glycine, serine, phenylalanine or tryptophan residue
  MISC_FEATURE (7)..(7): Xaa is leucine, asparagine, aspartate, threonine, tryptophan, tyrosine, valine, alanine, or histidine residue
  MISC_FEATURE (8)..(8): Xaa is aspartate, serine, asparagine or glutamine residue
  MISC_FEATURE (9)..(9): Xaa is alanine or proline residue
XXXXXXXXXFDI

SEQ ID NO: 51 - protein, 14 aa, Artificial Sequence
  Synthetic Polypeptide
  MISC_FEATURE: heavy chain CDR3
  MISC_FEATURE (1)..(1): Xaa is alanine or no residue
  MISC_FEATURE (2)..(2): Xaa is glutamate, tryosine or glycine or no residue
  MISC_FEATURE (3)..(3): Xaa is serine, asparagine, tryptophan, glutamate or no residue
  MISC_FEATURE (4)..(4): Xaa is aspartate, glycine, serine, or valine residue, or no residue
  MISC_FEATURE (5)..(5): Xaa is serine, glycine, or aspartate residue, or no residue
  MISC_FEATURE (6)..(6): Xaa is glycine, phenylalanine, aspartate, serine, tryptophan, or tyrosine residue, or no residue
  MISC_FEATURE (7)..(7): Xaa is a tyrosine, tryptophan, serine, or aspartate residue, or no residue
  MISC_FEATURE (8)..(8): Xaa is aspartate, arginine, serine, glycine, tyrosine, or tryptophan residue
  MISC_FEATURE (9)..(9): Xaa is tyrosine, isoleucine, leucine, phenylalanine, or lysine residue
  MISC_FEATURE (10)..(10): Xaa is tyrosine, phenylalanine, aspartate, or glycine residue
  MISC_FEATURE (11)..(11): Xaa is glycine, tyrosine, or asparagine residue
XXXXXXXXXXXMDV

SEQ ID NO: 52 - protein, 11 aa, Artificial Sequence
  Synthetic Polypeptide
  MISC_FEATURE: heavy chain CDR3
  MISC_FEATURE (1)..(1): Xaa is aspartate or valine residue, or no residue
  MISC_FEATURE (2)..(2): Xaa is glycine, tyrosine, arginine, or aspartate residue, or no residue,
  MISC_FEATURE (3)..(3): Xaa is asparagine, leucine, glycine, isoleucine, serine, valine, phenylalanine, or tyrosine residue, or no residue
  MISC_FEATURE (4)..(4): Xaa is leucine, serine, tryptophan, alanine, tyrosine, isoleucine, glycine, or aspartate residue, or no residue
  MISC_FEATURE (5)..(5): Xaa is glycine, alanine, tyrosine, serine, aspartate, or leucine residue
  MISC_FEATURE (6)..(6): Xaa is valine, alanine, glycine, threonine, proline, histidine, or glutamine residue
  MISC_FEATURE (7)..(7): Xaa is glutamate, glycine, serine, aspartate, glycine, valine, tryptophan, histidine, or arginine residue
  MISC_FEATURE (8)..(8): Xaa is glutamine, alanine, glycine, tyrosine, proline, leucine, aspartate, or serine residue
  MISC_FEATURE (9)..(9): Xaa is an amino acid with a nonpolar side chain
  MISC_FEATURE (10)..(10): Xaa is aspartate or alanine residue
XXXXXXXXXXY

SEQ ID NO: 53 - protein, 14 aa, Artificial Sequence
  Synthetic Polypeptide
  MISC_FEATURE: Heavy chain CDR3
  MISC_FEATURE (1)..(1): Xaa is glycine residue, or no residue
  MISC_FEATURE (2)..(2): Xaa is proline residue, or no residue
  MISC_FEATURE (3)..(3): Xaa is arginine or aspartate residue, or no residue
  MISC_FEATURE (4)..(4): Xaa is histidine or proline residue
  MISC_FEATURE (5)..(5): Xaa is arginine or glycine residue
  MISC_FEATURE (6)..(6): Xaa is arginine, serine, or phenylalanine residue
  MISC_FEATURE (7)..(7): Xaa is aspartate or serine residue
  MISC_FEATURE (8)..(8): Xaa is glycine, tryptophan, or tyrosine residue
  MISC_FEATURE (9)..(9): Xaa is tyrosine or alanine residue
  MISC_FEATURE (10)..(10): Xaa is asparagine or tryptophan residue
  MISC_FEATURE (14)..(14): Xaa is asparagine or leucine residue
XXXXXXXXXXYFDX

SEQ ID NO: 54 - protein, 15 aa, Artificial Sequence
  Synthetic Polypeptide
  MISC_FEATURE: heavy chain CDR3
  MISC_FEATURE (1)..(1): Xaa is phenylalanine residue, or no residue
  MISC_FEATURE (2)..(2): Xaa is asparagine or glycine residue, or no residue
  MISC_FEATURE (3)..(3): Xaa is tyrosine or a leucine residue, or no residue
  MISC_FEATURE (4)..(4): Xaa is a tyrosine or glycine residue, or no residue
  MISC_FEATURE (8)..(8): Xaa is a glycine, serine, or valine residue
  MISC_FEATURE (9)..(9): Xaa is tyrosine, phenylalanine, tryptophan, or glutamine residue, or no residue
  MISC_FEATURE (10)..(10): Xaa is tyrosine, glycine, or isoleucine residue, or no residue
  MISC_FEATURE (11)..(11): Xaa is tyrosine, leucine, or glycine residue, or no residue
  MISC_FEATURE (12)..(12): Xaa is methionine,  glycine, or phenylalanine residue, or no residue
  MISC_FEATURE (13)..(13): Xaa is  aspartate or methionine residue, or no residue
  MISC_FEATURE (14)..(14): Xaa is a valine, aspartate, or tyrosine residue, or no residue
  MISC_FEATURE (15)..(15): Xaa is a valine residue, or no residue
XXXXDSSXXXXXXXX

SEQ ID NO: 55 - protein, 16 aa, Artificial Sequence
  Synthetic Polypeptide
  MISC_FEATURE: Light Chain CDR1
RSSQSLLHSSGYNYLD

SEQ ID NO: 56 - protein, 16 aa, Artificial Sequence
  Synthetic Polypeptide
  MISC_FEATURE: Light Chain CDR1
RSSQSLLHSNGYNYLN

SEQ ID NO: 57 - protein, 16 aa, Artificial Sequence
  Synthetic Polypeptide
  MISC_FEATURE: Light Chain CDR1
RSSQSLLHSHGYNYLD

SEQ ID NO: 58 - protein, 16 aa, Artificial Sequence
  Synthetic Polypeptide
RSSQSLLHSNGYTYLD

SEQ ID NO: 59 - protein, 16 aa, Artificial Sequence
  Synthetic Polypeptide
  MISC_FEATURE: Light Chain CDR1
RSSQSLLHSNGYNFLD

SEQ ID NO: 60 - protein, 16 aa, Artificial Sequence
  Synthetic Polypeptide
  MISC_FEATURE: Light Chain CDR1
RSSQSLLHTNGYNYLD

SEQ ID NO: 61 - protein, 16 aa, Artificial Sequence
  Synthetic Polypeptide
  MISC_FEATURE: Light Chain CDR1
RSSQSLLHTNGYDYLD

SEQ ID NO: 62 - protein, 13 aa, Artificial Sequence
  Synthetic Polypeptide
  MISC_FEATURE: Light Chain CDR1
TGSGGNIASNYVQ

SEQ ID NO: 63 - protein, 13 aa, Artificial Sequence
  Synthetic Polypeptide
  MISC_FEATURE: Light Chain CDR1
TRSSGSIASNYVQ

SEQ ID NO: 64 - protein, 13 aa, Artificial Sequence
  Synthetic Polypeptide
  MISC_FEATURE: Light Chain CDR1
TRSSGDIDNNYVQ

SEQ ID NO: 65 - protein, 16 aa, Artificial Sequence
  Synthetic Polypeptide
  MISC_FEATURE: Light Chain CDR1
TRNSGSIASNFVQWYQ

SEQ ID NO: 66 - protein, 12 aa, Artificial Sequence
  Synthetic Polypeptide
  MISC_FEATURE: Light Chain CDR1
RASQTISSSHLA

SEQ ID NO: 67 - protein, 11 aa, Artificial Sequence
  Synthetic Polypeptide
  MISC_FEATURE: Light Chain CDR1
RASQGISRWLA

SEQ ID NO: 68 - protein, 11 aa, Artificial Sequence
  Synthetic Polypeptide
  MISC_FEATURE: Light Chain CDR1
RASQGISSYLA

SEQ ID NO: 69 - protein, 11 aa, Artificial Sequence
  Synthetic Polypeptide
RASQSVYNYLA

SEQ ID NO: 70 - protein, 11 aa, Artificial Sequence
  Synthetic Polypeptide
  MISC_FEATURE: Light Chain CDR1
RASQSVGSNLA

SEQ ID NO: 71 - protein, 11 aa, Artificial Sequence
  Synthetic Polypeptide
  MISC_FEATURE: Light Chain CDR1
RSSQGIGYFLN

SEQ ID NO: 72 - protein, 11 aa, Artificial Sequence
  Synthetic Polypeptide
  MISC_FEATURE: Light Chain CDR1
RASQSPGIFLN

SEQ ID NO: 73 - protein, 11 aa, Artificial Sequence
  Synthetic Polypeptide
  MISC_FEATURE: Light Chain CDR1
SGDKLGDKYVG

SEQ ID NO: 74 - protein, 11 aa, Artificial Sequence
  Synthetic Polypeptide
  MISC_FEATURE: Light Chain CDR1
QGDSLRIYYTG

SEQ ID NO: 75 - protein, 7 aa, Artificial Sequence
  Synthetic Polypeptide
  MISC_FEATURE: Light Chain CDR2
LGSNRAS

SEQ ID NO: 76 - protein, 7 aa, Artificial Sequence
  Synthetic Polypeptide
  MISC_FEATURE: Light Chain CDR2
LGSYRAS

SEQ ID NO: 77 - protein, 7 aa, Artificial Sequence
  Synthetic Polypeptide
  MISC_FEATURE: Light Chain CDR2
LVSNRAS

SEQ ID NO: 78 - protein, 7 aa, Artificial Sequence
  Synthetic Polypeptide
  MISC_FEATURE: Light Chain CDR2
LGSNRDS

SEQ ID NO: 79 - protein, 7 aa, Artificial Sequence
  Synthetic Polypeptide
  MISC_FEATURE: Light Chain CDR2
LGSTRAS

SEQ ID NO: 80 - protein, 7 aa, Artificial Sequence
  Synthetic Polypeptide
  MISC_FEATURE: Light Chain CDR2
LGFNRAS

SEQ ID NO: 81 - protein, 7 aa, Artificial Sequence
  Synthetic Polypeptide
  MISC_FEATURE: Light Chain CDR2
AASTLQS

SEQ ID NO: 82 - protein, 7 aa, Artificial Sequence
  Synthetic Polypeptide
  MISC_FEATURE: Light Chain CDR2
AASGLQS

SEQ ID NO: 83 - protein, 7 aa, Artificial Sequence
  Synthetic Polypeptide
  MISC_FEATURE: Light Chain CDR2
ATSTLES

SEQ ID NO: 84 - protein, 7 aa, Artificial Sequence
  Synthetic Polypeptide
  MISC_FEATURE: Light Chain CDR2
EDNQRPS

SEQ ID NO: 85 - protein, 7 aa, Artificial Sequence
  Synthetic Polypeptide
  MISC_FEATURE: Light Chain CDR2
EDNRRPS

SEQ ID NO: 86 - protein, 7 aa, Artificial Sequence
  Synthetic Polypeptide
  MISC_FEATURE: Light Chain CDR2
QDNKRPS

SEQ ID NO: 87 - protein, 7 aa, Artificial Sequence
  Synthetic Polypeptide
  MISC_FEATURE: Light Chain CDR2
GKNNRPS

SEQ ID NO: 88 - protein, 7 aa, Artificial Sequence
  Synthetic Polypeptide
  MISC_FEATURE: Light Chain CDR2
DASRRAT

SEQ ID NO: 89 - protein, 7 aa, Artificial Sequence
  Synthetic Polypeptide
  MISC_FEATURE: Light Chain CDR2
DASNRAT

SEQ ID NO: 90 - protein, 7 aa, Artificial Sequence
  Synthetic Polypeptide
  MISC_FEATURE: Light Chain CDR2
GAGYRAT

SEQ ID NO: 91 - protein, 9 aa, Artificial Sequence
  Synthetic Polypeptide
  MISC_FEATURE: Light Chain CDR3
MQALQTPLT

SEQ ID NO: 92 - protein, 9 aa, Artificial Sequence
  Synthetic Polypeptide
  MISC_FEATURE: Light Chain CDR3
MQAFQTPLT

SEQ ID NO: 93 - protein, 9 aa, Artificial Sequence
  Synthetic Polypeptide
  MISC_FEATURE: Light Chain CDR3
MQALQTPIT

SEQ ID NO: 94 - protein, 9 aa, Artificial Sequence
  Synthetic Polypeptide
  MISC_FEATURE: Light Chain CDR3
MQALQTPYT

SEQ ID NO: 95 - protein, 9 aa, Artificial Sequence
  Synthetic Polypeptide
  MISC_FEATURE: Light Chain CDR3
MQALQTPFT

SEQ ID NO: 96 - protein, 9 aa, Artificial Sequence
  Synthetic Polypeptide
  MISC_FEATURE: Light Chain CDR3
MQALQTPHT

SEQ ID NO: 97 - protein, 9 aa, Artificial Sequence
  Synthetic Polypeptide
  MISC_FEATURE: Light Chain CDR3
MQALQTPNT

SEQ ID NO: 98 - protein, 9 aa, Artificial Sequence
  Synthetic Polypeptide
  MISC_FEATURE: Light Chain CDR3
MQALQTPLA

SEQ ID NO: 99 - protein, 9 aa, Artificial Sequence
  Synthetic Polypeptide
  MISC_FEATURE: Light Chain CDR3
MQGLQTPLT

SEQ ID NO: 100 - protein, 9 aa, Artificial Sequence
  Synthetic Polypeptide
  MISC_FEATURE: Light Chain CDR3
MQALEMPLT

SEQ ID NO: 101 - protein, 9 aa, Artificial Sequence
  Synthetic Polypeptide
  MISC_FEATURE: Light Chain CDR3
MEALQTPFT

SEQ ID NO: 102 - protein, 9 aa, Artificial Sequence
  Synthetic Polypeptide
MQTLQTPLS

SEQ ID NO: 103 - protein, 9 aa, Artificial Sequence
  Synthetic Polypeptide
  MISC_FEATURE: Light Chain CDR3
MQGTHWPLT

SEQ ID NO: 104 - protein, 9 aa, Artificial Sequence
  Synthetic Polypeptide
  MISC_FEATURE: Light Chain CDR3
MQSLEVPFT

SEQ ID NO: 105 - protein, 9 aa, Artificial Sequence
  Synthetic Polypeptide
  MISC_FEATURE: Light Chain CDR3
MQATHWPYT

SEQ ID NO: 106 - protein, 9 aa, Artificial Sequence
  Synthetic Polypeptide
  MISC_FEATURE: Light Chain CDR3
MQGTHWPYT

SEQ ID NO: 107 - protein, 9 aa, Artificial Sequence
  Synthetic Polypeptide
  MISC_FEATURE: Light Chain CDR3
QQRNNWPLT

SEQ ID NO: 108 - protein, 9 aa, Artificial Sequence
  Synthetic Polypeptide
  MISC_FEATURE: Light Chain CDR3
QQRSNWPLT

SEQ ID NO: 109 - protein, 9 aa, Artificial Sequence
  Synthetic Polypeptide
  MISC_FEATURE: Light Chain CDR3
QQSNSVPLT

SEQ ID NO: 110 - protein, 9 aa, Artificial Sequence
  Synthetic Polypeptide
  MISC_FEATURE: Light Chain CDR3
QQLNSYPLT

SEQ ID NO: 111 - protein, 9 aa, Artificial Sequence
  Synthetic Polypeptide
  MISC_FEATURE: Light Chain CDR3
QQSHSPPYT

SEQ ID NO: 112 - protein, 9 aa, Artificial Sequence
  Synthetic Polypeptide
  MISC_FEATURE: Light Chain CDR3
QQASSFPIT

SEQ ID NO: 113 - protein, 10 aa, Artificial Sequence
  Synthetic Polypeptide
  MISC_FEATURE: Light Chain CDR3
QSYDSSNQRV

SEQ ID NO: 114 - protein, 9 aa, Artificial Sequence
  Synthetic Polypeptide
  MISC_FEATURE: Light Chain CDR3
QSYDPYNRV

SEQ ID NO: 115 - protein, 9 aa, Artificial Sequence
  Synthetic Polypeptide
  MISC_FEATURE: Light Chain CDR3
QSYDSSNVV

SEQ ID NO: 116 - protein, 9 aa, Artificial Sequence
  Synthetic Polypeptide
  MISC_FEATURE: Light Chain CDR3
QSYQSDNWV

SEQ ID NO: 117 - protein, 9 aa, Artificial Sequence
  Synthetic Polypeptide
  MISC_FEATURE: Light Chain CDR3
QSYDSANVI

SEQ ID NO: 118 - protein, 8 aa, Artificial Sequence
  Synthetic Polypeptide
  MISC_FEATURE: Light Chain CDR3
QAWDSGTV

SEQ ID NO: 119 - protein, 9 aa, Artificial Sequence
  Synthetic Polypeptide
  MISC_FEATURE: Light Chain CDR3
QHYGSSLRT

SEQ ID NO: 120 - protein, 10 aa, Artificial Sequence
  Synthetic Polypeptide
  MISC_FEATURE: Light Chain CDR3
NSRDITGVHR

SEQ ID NO: 121 - protein, 6 aa, Artificial Sequence
  Synthetic Polypeptide
  MISC_FEATURE: Heavy Chain CDR1
SSNWWS

SEQ ID NO: 122 - protein, 5 aa, Artificial Sequence
  Synthetic Polypeptide
  MISC_FEATURE: Heavy Chain CDR1
SNWWS

SEQ ID NO: 123 - protein, 6 aa, Artificial Sequence
  Synthetic Polypeptide
  MISC_FEATURE: Heavy Chain CDR1
SNIWWS

SEQ ID NO: 124 - protein, 5 aa, Artificial Sequence
  Synthetic Polypeptide
  MISC_FEATURE: Heavy Chain CDR1
GYYWS

SEQ ID NO: 125 - protein, 5 aa, Artificial Sequence
  Synthetic Polypeptide
  MISC_FEATURE: Heavy Chain CDR1
NYDWS

SEQ ID NO: 126 - protein, 5 aa, Artificial Sequence
  Synthetic Polypeptide
  MISC_FEATURE: Heavy Chain CDR1
NYYWS

SEQ ID NO: 127 - protein, 5 aa, Artificial Sequence
  Synthetic Polypeptide
  MISC_FEATURE: Heavy Chain CDR1
DFYWS

SEQ ID NO: 128 - protein, 5 aa, Artificial Sequence
  Synthetic Polypeptide
  MISC_FEATURE: Heavy Chain CDR1
SYAMS

SEQ ID NO: 129 - protein, 5 aa, Artificial Sequence
  Synthetic Polypeptide
  MISC_FEATURE: Heavy Chain CDR1
SYAIS

SEQ ID NO: 130 - protein, 5 aa, Artificial Sequence
  Synthetic Polypeptide
  MISC_FEATURE: Heavy Chain CDR1
SYGMH

SEQ ID NO: 131 - protein, 5 aa, Artificial Sequence
  Synthetic Polypeptide
  MISC_FEATURE: Heavy Chain CDR1
SYAMH

SEQ ID NO: 132 - protein, 5 aa, Artificial Sequence
  Synthetic Polypeptide
  MISC_FEATURE: Heavy Chain CDR1
SHGMH

SEQ ID NO: 133 - protein, 7 aa, Artificial Sequence
  Synthetic Polypeptide
  MISC_FEATURE: Heavy Chain CDR1
SSSYYWG

SEQ ID NO: 134 - protein, 16 aa, Artificial Sequence
  Synthetic Polypeptide
  MISC_FEATURE: Heavy Chain CDR2
EIYHSGSTNYNPSLKS

SEQ ID NO: 135 - protein, 16 aa, Artificial Sequence
  Synthetic Polypeptide
  MISC_FEATURE: Heavy Chain CDR2
EIYHSGSTNYNPSLES

SEQ ID NO: 136 - protein, 16 aa, Artificial Sequence
  Synthetic Polypeptide
  MISC_FEATURE: Heavy Chain CDR2
EINHSGSTNYNPSLKS

SEQ ID NO: 137 - protein, 16 aa, Artificial Sequence
  Synthetic Polypeptide
  MISC_FEATURE: Heavy Chain CDR2
EVYHSGSTNYNPSLKS

SEQ ID NO: 138 - protein, 16 aa, Artificial Sequence
  Synthetic Polypeptide
  MISC_FEATURE: Heavy Chain CDR2
EINHSGSTNYNRSLKS

SEQ ID NO: 139 - protein, 16 aa, Artificial Sequence
  Synthetic Polypeptide
  MISC_FEATURE: Heavy Chain CDR2
YIYYSGSTYYNPSLKS

SEQ ID NO: 140 - protein, 16 aa, Artificial Sequence
  Synthetic Polypeptide
  MISC_FEATURE: Heavy Chain CDR2
TIYSSGSTYYSPSLKS

SEQ ID NO: 141 - protein, 16 aa, Artificial Sequence
  Synthetic Polypeptide
  MISC_FEATURE: Heavy Chain CDR2
SIYYSGSTYYNPSLKS

SEQ ID NO: 142 - protein, 16 aa, Artificial Sequence
  Synthetic Polypeptide
  MISC_FEATURE: Heavy Chain CDR2
YISDSGNTNYNPSLKS

SEQ ID NO: 143 - protein, 16 aa, Artificial Sequence
  Synthetic Polypeptide
  MISC_FEATURE: Heavy Chain CDR2
EVNPRGSTNYNPSLKS

SEQ ID NO: 144 - protein, 17 aa, Artificial Sequence
  Synthetic Polypeptide
  MISC_FEATURE: Heavy Chain CDR2
TISGSGGSTYYADSVKG

SEQ ID NO: 145 - protein, 17 aa, Artificial Sequence
  Synthetic Polypeptide
  MISC_FEATURE: Heavy Chain CDR2
AISGSGGSTYYADSVKG

SEQ ID NO: 146 - protein, 17 aa, Artificial Sequence
  Synthetic Polypeptide
  MISC_FEATURE: Heavy Chain CDR2
TISSNGDSTYYADSVKG

SEQ ID NO: 147 - protein, 17 aa, Artificial Sequence
  Synthetic Polypeptide
  MISC_FEATURE: Heavy Chain CDR2
VISYDGSNKYYADSVKG

SEQ ID NO: 148 - protein, 17 aa, Artificial Sequence
  Synthetic Polypeptide
  MISC_FEATURE: Heavy Chain CDR2
YISSSSSTIYYADSVKG

SEQ ID NO: 149 - protein, 17 aa, Artificial Sequence
  Synthetic Polypeptide
  MISC_FEATURE: Heavy Chain CDR2
RIIPILGIANYAQKFQG

SEQ ID NO: 150 - protein, 17 aa, Artificial Sequence
  Synthetic Polypeptide
  MISC_FEATURE: Heavy Chain CDR2
WINAGNGNTKYSQKFQG

SEQ ID NO: 151 - protein, 17 aa, Artificial Sequence
  Synthetic Polypeptide
  MISC_FEATURE: Heavy Chain CDR2
IINPSGGSTSYAQKFQG

SEQ ID NO: 152 - protein, 11 aa, Artificial Sequence
  Synthetic Polypeptide
  MISC_FEATURE: Heavy Chain CDR3
YSSSRNDAFDI

SEQ ID NO: 153 - protein, 9 aa, Artificial Sequence
  Synthetic Polypeptide
  MISC_FEATURE: Heavy Chain CDR3
DGQLDAFDI

SEQ ID NO: 154 - protein, 9 aa, Artificial Sequence
  Synthetic Polypeptide
  MISC_FEATURE: Heavy Chain CDR3
WSYLDAFDI

SEQ ID NO: 155 - protein, 9 aa, Artificial Sequence
  Synthetic Polypeptide
  MISC_FEATURE: Heavy Chain CDR3
ANRDDAFDI

SEQ ID NO: 156 - protein, 12 aa, Artificial Sequence
  Synthetic Polypeptide
  MISC_FEATURE: Heavy Chain CDR3
EGNRTVTSAFDI

SEQ ID NO: 157 - protein, 10 aa, Artificial Sequence
  Synthetic Polypeptide
  MISC_FEATURE: Heavy Chain CDR3
WTGRTDAFDI

SEQ ID NO: 158 - protein, 9 aa, Artificial Sequence
  Synthetic Polypeptide
  MISC_FEATURE: Heavy Chain CDR3
QGALDAFDI

SEQ ID NO: 159 - protein, 11 aa, Artificial Sequence
  Synthetic Polypeptide
  MISC_FEATURE: Heavy Chain CDR3
SSSWYWNAFDI

SEQ ID NO: 160 - protein, 8 aa, Artificial Sequence
  Synthetic Polypeptide
  MISC_FEATURE: Heavy Chain CDR3
SGYDAFDI

SEQ ID NO: 161 - protein, 8 aa, Artificial Sequence
  Synthetic Polypeptide
  MISC_FEATURE: Heavy Chain CDR3
ASVDAFDI

SEQ ID NO: 162 - protein, 9 aa, Artificial Sequence
  Synthetic Polypeptide
  MISC_FEATURE: Heavy Chain CDR3
LSFADPFDI

SEQ ID NO: 163 - protein, 10 aa, Artificial Sequence
  Synthetic Polypeptide
EEVWLQAFDI

SEQ ID NO: 164 - protein, 8 aa, Artificial Sequence
  Synthetic Polypeptide
  MISC_FEATURE: Heavy Chain CDR3
IRYDAFDI

SEQ ID NO: 165 - protein, 9 aa, Artificial Sequence
  Synthetic Polypeptide
  MISC_FEATURE: Heavy Chain CDR3
TAAHDAFDI

SEQ ID NO: 166 - protein, 10 aa, Artificial Sequence
  Synthetic Polypeptide
  MISC_FEATURE: Heavy Chain CDR3
DRWRYDAFDI

SEQ ID NO: 167 - protein, 9 aa, Artificial Sequence
  Synthetic Polypeptide
  MISC_FEATURE: Heavy Chain CDR3
FWDYYGMDV

SEQ ID NO: 168 - protein, 7 aa, Artificial Sequence
  Synthetic Polypeptide
  MISC_FEATURE: Heavy Chain CDR3
EKSGMDV

SEQ ID NO: 169 - protein, 8 aa, Artificial Sequence
  Synthetic Polypeptide
  MISC_FEATURE: Heavy Chain CDR3
DRYYGMDV

SEQ ID NO: 170 - protein, 9 aa, Artificial Sequence
  Synthetic Polypeptide
  MISC_FEATURE: Heavy Chain CDR3
DYDIFGMDV

SEQ ID NO: 171 - protein, 13 aa, Artificial Sequence
  Synthetic Polypeptide
  MISC_FEATURE: Heavy Chain CDR3
ERGSGWSLDNMDV

SEQ ID NO: 172 - protein, 10 aa, Artificial Sequence
  Synthetic Polypeptide
  MISC_FEATURE: Heavy Chain CDR3
DSSGFYGMDV

SEQ ID NO: 173 - protein, 11 aa, Artificial Sequence
  Synthetic Polypeptide
  MISC_FEATURE: Heavy Chain CDR3
DGGYYYYGMDV

SEQ ID NO: 174 - protein, 9 aa, Artificial Sequence
  Synthetic Polypeptide
  MISC_FEATURE: Heavy Chain CDR3
HSYYYGMDV

SEQ ID NO: 175 - protein, 11 aa, Artificial Sequence
  Synthetic Polypeptide
  MISC_FEATURE: Heavy Chain CDR3
VSGYYYYGMDV

SEQ ID NO: 176 - protein, 14 aa, Artificial Sequence
  Synthetic Polypeptide
  MISC_FEATURE: Heavy Chain CDR3
AYSSGWYDYYGMDV

SEQ ID NO: 177 - protein, 11 aa, Artificial Sequence
  Synthetic Polypeptide
  MISC_FEATURE: Heavy Chain CDR3
DSSSWYYGMDV

SEQ ID NO: 178 - protein, 13 aa, Artificial Sequence
  Synthetic Polypeptide
  MISC_FEATURE: Heavy Chain CDR3
GSGSYYDYYYMDV

SEQ ID NO: 179 - protein, 7 aa, Artificial Sequence
  Synthetic Polypeptide
  MISC_FEATURE: Heavy Chain CDR3
DKGYMDV

SEQ ID NO: 180 - protein, 7 aa, Artificial Sequence
  Synthetic Polypeptide
  MISC_FEATURE: Heavy Chain CDR3
GVEQIDY

SEQ ID NO: 181 - protein, 9 aa, Artificial Sequence
  Synthetic Polypeptide
  MISC_FEATURE: Heavy Chain CDR3
NLAAGAVAY

SEQ ID NO: 182 - protein, 9 aa, Artificial Sequence
  Synthetic Polypeptide
  MISC_FEATURE: Heavy Chain CDR3
LSYGSGVDY

SEQ ID NO: 183 - protein, 10 aa, Artificial Sequence
  Synthetic Polypeptide
  MISC_FEATURE: Heavy Chain CDR3
GGWYGDYFDY

SEQ ID NO: 184 - protein, 10 aa, Artificial Sequence
  Synthetic Polypeptide
  MISC_FEATURE: Heavy Chain CDR3
GIAAAGQGDY

SEQ ID NO: 185 - protein, 10 aa, Artificial Sequence
  Synthetic Polypeptide
  MISC_FEATURE: Heavy Chain CDR3
YSYGTVGIDY

SEQ ID NO: 186 - protein, 8 aa, Artificial Sequence
  Synthetic Polypeptide
  MISC_FEATURE: Heavy Chain CDR3
IGPGGFDY

SEQ ID NO: 187 - protein, 9 aa, Artificial Sequence
  Synthetic Polypeptide
  MISC_FEATURE: Heavy Chain CDR3
VGSGWYVDY

SEQ ID NO: 188 - protein, 7 aa, Artificial Sequence
  Synthetic Polypeptide
  MISC_FEATURE: Heavy Chain CDR3
DHGPFDY

SEQ ID NO: 189 - protein, 11 aa, Artificial Sequence
  Synthetic Polypeptide
  MISC_FEATURE: Heavy Chain CDR3
DRFGSGHLPDY

SEQ ID NO: 190 - protein, 11 aa, Artificial Sequence
  Synthetic Polypeptide
  MISC_FEATURE: Heavy Chain CDR3
VGYSSGRDVDY

SEQ ID NO: 191 - protein, 7 aa, Artificial Sequence
  Synthetic Polypeptide
  MISC_FEATURE: Heavy Chain CDR3
STWSLDY

SEQ ID NO: 192 - protein, 8 aa, Artificial Sequence
  Synthetic Polypeptide
  MISC_FEATURE: Heavy Chain CDR3
DLTGSLDY

SEQ ID NO: 193 - protein, 9 aa, Artificial Sequence
  Synthetic Polypeptide
  MISC_FEATURE: Heavy Chain CDR3
DSSGQYFDY

SEQ ID NO: 194 - protein, 14 aa, Artificial Sequence
  Synthetic Polypeptide
  MISC_FEATURE: Heavy Chain CDR3
GPRPGRDGYNYFDN

SEQ ID NO: 195 - protein, 11 aa, Artificial Sequence
  Synthetic Polypeptide
  MISC_FEATURE: Heavy Chain CDR3
HRSSWAWYFDL

SEQ ID NO: 196 - protein, 12 aa, Artificial Sequence
  Synthetic Polypeptide
  MISC_FEATURE: Heavy Chain CDR3
DHRFDYAWYFDL

SEQ ID NO: 197 - protein, 8 aa, Artificial Sequence
  Synthetic Polypeptide
  MISC_FEATURE: Heavy Chain CDR3
FNYYDSSV

SEQ ID NO: 198 - protein, 10 aa, Artificial Sequence
  Synthetic Polypeptide
  MISC_FEATURE: Heavy Chain CDR3
GLGDSSGYIL

SEQ ID NO: 199 - protein, 8 aa, Artificial Sequence
  Synthetic Polypeptide
  MISC_FEATURE: Heavy Chain CDR3
YRSFGESY

SEQ ID NO: 200 - protein, 12 aa, Artificial Sequence
  Synthetic Polypeptide
  MISC_FEATURE: Heavy Chain CDR3
VGYSHGEEVLDV

SEQ ID NO: 201 - protein, 10 aa, Artificial Sequence
  Synthetic Polypeptide
  MISC_FEATURE: Heavy Chain CDR3
ARGYSSPFDP

SEQ ID NO: 202 - protein, 112 aa, Artificial Sequence
  Synthetic Polypeptide
  MISC_FEATURE: light chain variable region L1
DVVMTQSPLSLPVTPGEPASISCRSSQSLLHSSGYNYLDWYLQKPGQSPQLLIYLGSNRASGVPDRFSGSGSGTDFTLKISRVEAEDVGVYYCMQALQTPITFGQGTRLEIK

SEQ ID NO: 203 - protein, 117 aa, Artificial Sequence
  Synthetic Polypeptide
  MISC_FEATURE: heavy chain variable region H1
EVQLVETGPGLVKPSGTLSLTCAVSGGSISSSNWWSWVRQPPGKGLEWIGEIYHSGSTNYNPSLKSRVTISVDKSKNQFSLKLSSVTAADTAVYYCARFNYYDSSVWGQGTLVTVSS

SEQ ID NO: 204 - protein, 109 aa, Artificial Sequence
  Synthetic Polypeptide
  MISC_FEATURE: light chain variable region L2
MTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGQGTRLEIK

SEQ ID NO: 205 - protein, 116 aa, Artificial Sequence
  Synthetic Polypeptide
  MISC_FEATURE: heavy chain variable region H2
EVQLVETGPGLVKPSGTLSLTCAVSGGSISSSNWWSWVRQPPGKGLEWIGEIYHSGSTNYNPSLKSRVTISVDKSKNQFSLKLSSVTAADTAVYYCARGVEQIDYWGQGTLVTVSS

SEQ ID NO: 206 - protein, 112 aa, Artificial Sequence
  Synthetic Polypeptide
  MISC_FEATURE: Light chain variable region L3
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 207 - protein, 118 aa, Artificial Sequence
  Synthetic Polypeptide
  MISC_FEATURE: heavy chain variable region H3
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCAKNLAAGAVAYWGQGTLVTVSS

SEQ ID NO: 208 - protein, 112 aa, Artificial Sequence
  Synthetic Polypeptide
  MISC_FEATURE: light chain variable region L4
EIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGGGTKVEIK

SEQ ID NO: 209 - protein, 117 aa, Artificial Sequence
  Synthetic Polypeptide
  MISC_FEATURE: heavy chain variable region H4
QVQLQQWGAGLLKPSETLSLTCAVSGGSFSGYYWSWIRQPPGKGLEWIGEINHSGSTNYNRSLKSRVTISVDTSKNQFSLKLSSVTAADTAVYYCARLSYGSGVDYWGQGTLVTVSS

SEQ ID NO: 210 - protein, 112 aa, Artificial Sequence
  Synthetic Polypeptide
  MISC_FEATURE: light chain variable region L5
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGPGTKVDIK

SEQ ID NO: 211 - protein, 120 aa, Artificial Sequence
  Synthetic Polypeptide
  MISC_FEATURE: heavy chain variable region H5
QLQLQESGPGLVKPSQTLSLTCTVSGGSISSSNWWSWVRQPPGKGLEWIGEIYHSGSTNYNPSLKSRVTISVDKSKNQFSLKLSSVTAADTAVYYCARYSSSRNDAFDIWGQGTMVTVSS

SEQ ID NO: 212 - protein, 112 aa, Artificial Sequence
  Synthetic Polypeptide
  MISC_FEATURE: light chain variable region L6
DVVMTQSPLSLAVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 213 - protein, 118 aa, Artificial Sequence
  Synthetic Polypeptide
  MISC_FEATURE: heavy chain variable region H6
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARDGQLDAFDIWGQGTMVTVSS

SEQ ID NO: 214 - protein, 112 aa, Artificial Sequence; identical to SEQ ID NO: 206
  Synthetic Polypeptide
  MISC_FEATURE: light chain variable region L7

SEQ ID NO: 215 - protein, 118 aa, Artificial Sequence
  Synthetic Polypeptide
  MISC_FEATURE: heavy chain variable region H7
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARFWDYYGMDVWGQGTTVTVSS

SEQ ID NO: 216 - protein, 112 aa, Artificial Sequence; identical to SEQ ID NO: 206
  Synthetic Polypeptide
  MISC_FEATURE: light chain variable region L8

SEQ ID NO: 217 - protein, 117 aa, Artificial Sequence
  Synthetic Polypeptide
  MISC_FEATURE: heavy chain variable region H8
QVQLQQWGPGLVKPSGTLSLTCAVSGGSISSSNWWSWVRQPPGKGLEWIGEIYHSGSTNYNPSLESRVTISVDKSKNQFSLKLSSVTAADTAVYYCARDRYYGMDVWGQGTTVTVSS

SEQ ID NO: 218 - protein, 112 aa, Artificial Sequence
  Synthetic Polypeptide
  MISC_FEATURE: light chain variable region L9
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGQGTRLEIK

SEQ ID NO: 219 - protein, 118 aa, Artificial Sequence
  Synthetic Polypeptide
  MISC_FEATURE: heavy chain variable region H9
EVQLVESGPGLVKPSGTLSLTCAVSGGSISSSNWWSWVRQPPGKGLEWIGYIYYSGSTYYNPSLKSRVTMSVDTSKNQFSLKLSSVTAADTAVYYCARWSYLDAFDIWGQGTMVTVSS

SEQ ID NO: 220 - protein, 112 aa, Artificial Sequence
  Synthetic Polypeptide
  MISC_FEATURE: light chain variable region L10
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLAFGQGTKVEIK

SEQ ID NO: 221 - protein, 118 aa, Artificial Sequence
  Synthetic Polypeptides
  MISC_FEATURE: heavy chain variable region H10
EVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCARDYDIFGMDVWGQGTTVTVSS

SEQ ID NO: 222 - protein, 112 aa, Artificial Sequence
  Synthetic Polypeptide
  MISC_FEATURE: light chain variable region L11
EIVLTQSPLSLPVTPGEPASISCRSSQSLLHSNGYNYLNWYLQKPGQSPQLLIYLGSNRASGVPDRFSASGSGTDFTLKISRVEAEDVGVYYCMQALQTPITFGQGTRLEIK

SEQ ID NO: 223 - protein, 118 aa, Artificial Sequence
  Synthetic Polypeptide
  MISC_FEATURE: heavy chain variable region H11
QLQLQESGPGLVKPSGTLSLTCAVSGGSISSSNWWSWVRQPPGKGLEWIGEIYHSGSTNYNPSLKSRVTISVDKSKNQSSLKLSSVTAADTAVYYCARANRDDAFDIWGQGTMVTVSS

SEQ ID NO: 224 - protein, 111 aa, Artificial Sequence
  Synthetic Polypeptide
  MISC_FEATURE: light chain variable region L12
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNQRVFGGGTKLTVL

SEQ ID NO: 225 - protein, 119 aa, Artificial Sequence
  Synthetic Polypeptide
  MISC_FEATURE: heavy chain variable region H12
EVQLVESGGGLVQPGGSLRLSCAASGFTFSSYAMSWVRQAPGKGLEWVSAISGSGGSTYYADSVKGRFTISRDNSKNTLYLQMNSLSADDTAVYFCASGGWYGDYFDYWGQGTLVTVSS

SEQ ID NO: 226 - protein, 112 aa, Artificial Sequence; identical to SEQ ID NO: 206
  Synthetic Polypeptide
  MISC_FEATURE: light chain variable region L13

SEQ ID NO: 227 - protein, 121 aa, Artificial Sequence
  Synthetic Polypeptide
  MISC_FEATURE: heavy chain variable region H13
QVQLQESGPGLVKPSETLSLTCTVSGGSISSSNWWSWVRQPPGKGLEWIGEIYHSGSTNYNPSLKSRVTISVDKSKNQFSLKLSSVTAADTAVYYCAREGNRTVTSAFDIWGQGTMVTVSS

SEQ ID NO: 228 - protein, 112 aa, Artificial Sequence; identical to SEQ ID NO: 206
  Synthetic Polypeptide
  MISC_FEATURE: light chain variable region L14

SEQ ID NO: 229 - protein, 119 aa, Artificial Sequence
  Synthetic Polypeptide
  MISC_FEATURE: heavy chain variable region H14
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGLGDSSGYILWGQGTMVTVSS

SEQ ID NO: 230 - protein, 112 aa, Artificial Sequence
  Synthetic Polypeptide
  MISC_FEATURE: light chain variable region L15
DVVMTQSPLSLPVTPGEPASISCRSSQSLLHSNGYNYLDWYLQKPGQSPQLLIYLGSYRASGVPDRFSASGSGTDFTLKISRVEAEDVGVYYCMQALQTPITFGQGTRLEIK

SEQ ID NO: 231 - protein, 119 aa, Artificial Sequence; identical to SEQ ID NO: 229
  Synthetic Polypeptide
  MISC_FEATURE: heavy chain variable region H15

SEQ ID NO: 232 - protein, 112 aa, Artificial Sequence [sequence omitted]

SEQ ID NO: 233 - protein, 119 aa, Artificial Sequence [sequence omitted]

SEQ ID NO: 234 - protein, 111 aa, Artificial Sequence
  Synthetic Polypeptide
  MISC_FEATURE: light chain variable region L17
EIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEI

SEQ ID NO: 235 - protein, 118 aa, Artificial Sequence
  Synthetic Polypeptide
  MISC_FEATURE: heavy chain variable region H17
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARQGALDAFDIWGQGTTVTVSS

SEQ ID NO: 236 - protein, 107 aa, Artificial Sequence
  Synthetic Polypeptide
  MISC_FEATURE: light chain variable region L18
DIQLTQSPSSVSASVGDRVTITCRASQGISRWLAWYQQKPGKAPRLLIYAASGLQSGVPSRFSGSGSGTDFTLTISNLQPEDFATYYCQQASSFPITFGQGTRLETK

SEQ ID NO: 237 - protein, 122 aa, Artificial Sequence
  Synthetic Polypeptide
  MISC_FEATURE: heavy chain variable region H18
QVQLVESGGGVVRPGGSLRLSCAASGFTFSSYAMSWVRQAPGKGLEWVSTISGSGGSTYYADSVKGRFTISRDNSKNTLYLQMNSLRAEDTAVYYCAKERGSGWSLDNMDVWGQGTTVTVSS

SEQ ID NO: 238 - protein, 112 aa, Artificial Sequence
  Synthetic Polypeptide
  MISC_FEATURE: light chain variable region L19
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 239 - protein, 119 aa, Artificial Sequence
  Synthetic Polypeptide
  MISC_FEATURE: heavy chain variable region H19
QVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCARDSSGFYGMDVWGQGTTVTVSS

SEQ ID NO: 240 - protein, 112 aa, Artificial Sequence
  Synthetic Polypeptide
  MISC_FEATURE: light chain variable region L20
DVVMTQSPLSLPVTPGEPASISCRSSQSLLHSNGYNYLDWYLQKPGQSPQLLIYLGSNRASGVPNRFSGSGSGTDFTLKISRVEAEDVGVYYCMQALQTPFTFGPGTKVDIK

SEQ ID NO: 241 - protein, 120 aa, Artificial Sequence
  Synthetic Polypeptide
  MISC_FEATURE: heavy chain variable region H20
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARSSSWYWNAFDIWGQGTMVTVSS

SEQ ID NO: 242 - protein, 112 aa, Artificial Sequence
  Synthetic Polypeptode
  MISC_FEATURE: light chain variable region L21
DVVMTQSPLSLPVTPGEPASISCRSSQSLLHSHGYNYLDWYLQKPGQSPQLLIYLGSYRASGVPDRFSGSGSGTDFTLKISRVEAEDVGVYYCMQSLEVPFTFGQGTKLEIK

SEQ ID NO: 243 - protein, 117 aa, Artificial Sequence
  Synthetic Polypeptide
  MISC_FEATURE: heavy chain variable region H21
QVQLQQWGPALVKPSGTLSLTCSVSGVSITSNIWWSWVRQSPGKGLEWIGEVYHSGSTNYNPSLKSRVTISVDKSKNQFSLKLSSVTAADTAVYYCAGYRSFGESYWGQGTLVTVSS

SEQ ID NO: 244 - protein, 107 aa, Artificial Sequence
  Synthetic Polypeptide
  MISC_FEATURE: light chain variable region L22
SSELTQDPAVSVALGQTVRITCQGDSLRIYYTGWYQQKPGQAPVLVLFGKNNRPSGIPDRFSGSHSGNTASLTITGAQAEDEADYYCNSRDITGVHRFGGGTKLTVL

SEQ ID NO: 245 - protein, 122 aa, Artificial Sequence
  Synthetic Polypeptide
  MISC_FEATURE: heavy chain variable region H22
QVQLQQWGAGLLKPSETLSLTCVVYGGSFSDFYWSWIRQPPGKGPEWIGEVNPRGSTNYNPSLKSRATISLDTSKNQFSLKLSSVTAADTAVYFCARGPRPGRDGYNYFDNWGQGTLVTVSS

SEQ ID NO: 246 - protein, 112 aa, Artificial Sequence
  Synthetic Polypeptide
  MISC_FEATURE: light chain variable region L23
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 247 - protein, 119 aa, Artificial Sequence
  Synthetic Polypeptide
  MISC_FEATURE: heavy chain variable region H23
QVQLQESGPGLVKPSETLSLTCTVSGGSISSSNWWSWVRQPPGKGLEWIGEIYHSGSTNYNPSLKSRVTISVDKSKNQFSLKLSSVTAADTAVYYCARGIAAAGQGDYWGQGTLVTVSS

SEQ ID NO: 248 - protein, 112 aa, Artificial Sequence
  Synthetic Polypeptide
  MISC_FEATURE: light chain variable region L24
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPNTFGGGTKVEIK

SEQ ID NO: 249 - protein, 121 aa, Artificial Sequence
  Synthetic Polypeptide
  MISC_FEATURE: heavy chain variable region H24
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDGGYYYYGMDVWGQGTTVTVSS

SEQ ID NO: 250 - protein, 112 aa, Artificial Sequence
  Synthetic Polypeptide
  MISC_FEATURE: light chain variable region L25
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGPGTKVDIK

SEQ ID NO: 251 - protein, 117 aa, Artificial Sequence
  Synthetic Polypeptide
  MISC_FEATURE: heavy chain variable region H25
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCASSGYDAFDIWGQGTTVTVSS

SEQ ID NO: 252 - protein, 112 aa, Artificial Sequence
  Synthetic Polypeptide
  MISC_FEATURE: light chain variable region L26
DVVMTQSPLSLPVTPGEPASISCRSSQSLLHSNGYTYLDWYLQKPGQSPQLLIYLGSNRASGVPDRFSGSGSGTDFTLKISRVEPEDVGVYYCMQALEMPLTFGGGTKVEIK

SEQ ID NO: 253 - protein, 119 aa, Artificial Sequence
  Synthetic Polypeptide
  MISC_FEATURE: heavy chain variable region H26
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARYSYGTVGIDYWGQGTLVTVSS

SEQ ID NO: 254 - protein, 107 aa, Artificial Sequence
  Synthetic Polypeptide
  MISC_FEATURE: light chain variable region L27
DIQLTQSPSFLSASVGDRVTITCRASQGISSYLAWYQQKPGKAPKLLIYAASTLQSGVPSRFSGSGSGTEFTLTISSLQPEDFATYYCQQLNSYPLTFGGGTKVEIK

SEQ ID NO: 255 - protein, 117 aa, Artificial Sequence
  Synthetic Polypeptide
  MISC_FEATURE: heavy chain variable region H27
EVQLVQSGGGVVQPGTSLRLSCAASGFSFRSHGMHWVRQAPGKGLEWVAVISYDGSNKYYADSVKGRFTISRDNSKNTLYLQMNSLRAEDTAVYYCATIGPGGFDYWGQGTLVTVSS

SEQ ID NO: 256 - protein, 105 aa, Artificial Sequence
  Synthetic Polypeptide
  MISC_FEATURE: light chain variable region L28
SYVLTQPPSVSVSPGQTASITCSGDKLGDKYVGWYQQKAGQAPVLVIYQDNKRPSGIPERFSGSNSGNTASLTISGTQAMDEADYYCQAWDSGTVFGGGTKLTVL

SEQ ID NO: 257 - protein, 119 aa, Artificial Sequence
  Synthetic Polypeptide
  MISC_FEATURE: heavy chain variable region H28
QVQLQESGPGLVKPSETLSLTCTVSGGSIRNYYWSWIRQPPGKGLEWIGYISDSGNTNYNPSLKSRVTISVDTSKNQFSLKLTSVTATDTAAYFCARHRSSWAWYFDLWGRGTLVTVSS

SEQ ID NO: 258 - protein, 112 aa, Artificial Sequence; identical to SEQ ID NO: 206
  Synthetic Polypeptide
  MISC_FEATURE: light chain variable region L29

SEQ ID NO: 259 - protein, 118 aa, Artificial Sequence
  Synthetic Polypeptide
  MISC_FEATURE: heavy chain variable region H29
QVQLQESGPGLVKPSETLSLTCAVSGGSISSSNWWSWVRQPPGKGLEWIGEIYHSGSTNYNPSLKSRVTISVDKSKNQFSLKLSSVTAADTAVYYCARVGSGWYVDYWGQGTLVTVSS

SEQ ID NO: 260 - protein, 112 aa, Artificial Sequence
  Synthetic Polypeptide
  MISC_FEATURE: light chain variable region L30
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMEALQTPFTFGPGTKVEIK

SEQ ID NO: 261 - protein, 120 aa, Artificial Sequence
  Synthetic Polypeptide
  MISC_FEATURE: heavy chain variable region H30
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARVSGYYYYGMDVWGQGTTVTVSS

SEQ ID NO: 262 - protein, 107 aa, Artificial Sequence
  Synthetic Polypeptide
  MISC_FEATURE: light chain variable region L31
DIQLTQSPSSLSASVGDRVTITCRSSQGIGYFLNWYQQEPGKAPKILISAASTLQSGVPSRFSGSGSGTDFTLSINNLQPADFATYYCQQSHSPPYTFGQGTKVEIK

SEQ ID NO: 263 - protein, 123 aa, Artificial Sequence
  Synthetic Polypeptide
  MISC_FEATURE: heavy chain variable region H31
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKAYSSGWYDYYGMDVWGQGTTVTVSS

SEQ ID NO: 264 - protein, 112 aa, Artificial Sequence; identical to SEQ ID NO: 206
  Synthetic Polypeptide
  MISC_FEATURE: light chain variable region L32

SEQ ID NO: 265 - protein, 117 aa, Artificial Sequence
  Synthetic Polypeptide
  MISC_FEATURE: heavy chain variable region H32
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARASVDAFDIWGQGTMVTVSS

SEQ ID NO: 266 - protein, 112 aa, Artificial Sequence
  Synthetic Polypeptide
  MISC_FEATURE: light chain variable region L33
EIVLTQSPLSLPVTPGEPASISCRSSQSLLHSNGYNYLDWYLQKPGQSPQLLMYLVSNRASGVPERFSGSGSGTDFTLKISRVEAEDVGVYYCMQTLQTPLSFGQGTKLEIK

SEQ ID NO: 267 - protein, 119 aa, Artificial Sequence; identical to SEQ ID NO: 229
  Synthetic Polypeptide
  MISC_FEATURE: heavy chain variable region H33

SEQ ID NO: 268 - protein, 112 aa, Artificial Sequence; identical to SEQ ID NO: 206
  Synthetic Polypeptide
  MISC_FEATURE: light chain variable region L34

SEQ ID NO: 269 - protein, 116 aa, Artificial Sequence
  Synthetic Polypeptide
  MISC_FEATURE: heavy chain variable region H34
QVQLQQSGPGLVKPSGTLSLTCAVSGGSISSSNWWSWVRQPPGKGLEWIGEIYHSGSTNYNPSLKSRVTISVDKSKNQFSLKLSSVTPEDTAVYYCARDHGPFDYWGRGTLVTVSS

SEQ ID NO: 270 - protein, 110 aa, Artificial Sequence
  Synthetic Polypeptide
  MISC_FEATURE: light chain variable region L35
NFMLTQPHSVSASPGKTVTISCTRSSGDIDNNYVQWYQQRPGNSPTNVIYEDNRRPSGVPDRFSGSIDSSSNSASLTISGLQPEDEADYYCQSYQSDNWVFGGGTKVTVL

SEQ ID NO: 271 - protein, 120 aa, Artificial Sequence
  Synthetic Polypeptide
  MISC_FEATURE: heavy chain variable region H35
QVQLVQSGGGVVQPGRSLRLSCAASGFAFSSYGMHWVRQAPGKGLEWVSYISSSSSTIYYADSVKGRFTISRDNSKNTLYLQMNSLRAEDTAVYYCARDRFGSGHLPDYWGQGTLVTVSS

SEQ ID NO: 272 - protein, 110 aa, Artificial Sequence
  Synthetic Polypeptide
  MISC_FEATURE: light chain variable region L36
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVVFGGGTKLTVL

SEQ ID NO: 273 - protein, 119 aa, Artificial Sequence
  Synthetic Polypeptide
  MISC_FEATURE: heavy chain variable region H36
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVGYSSGRDVDYWGQGTLVTVSS

SEQ ID NO: 274 - protein, 112 aa, Artificial Sequence
  Synthetic Polypeptide
  MISC_FEATURE: light chain variable region L37
DVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPYTFGQGTRLEIK

SEQ ID NO: 275 - protein, 120 aa, Artificial Sequence
  Synthetic Polypeptide
  MISC_FEATURE: heavy chain variable region H37
EVQLVESGPGLVKPSGTLSLTCAVSGGSISSSNWWSWIRQPPGKGLEWIGEIYHSGSTNYNPSLKSRVTISVDKSKNQFSLKLSSVTAADTAVYYCARDSSSWYYGMDVWGQGTTVTVSS

SEQ ID NO: 276 - protein, 112 aa, Artificial Sequence
  Synthetic Polypeptide
  MISC_FEATURE: light chain variable region L38
DVVMTQSPLSLPVTPGESASISCRSSQSLLHSNGYNFLDWYLQKPGQSPQLLIYLGSNRASGVPDRFSGSGSGTDFTLKISRVEAEDVGVYYCMQALQTPLTFGGGTKVEIK

SEQ ID NO: 277 - protein, 116 aa, Artificial Sequence
  Synthetic Polypeptide
  MISC_FEATURE: heavy chain variable region H38
EVQLVESGPGLVKPSETLSLTCAVSGGSISSSNWWSWVRQPPGKGLEWIGEIYHSGSTNYNPSLKSRVTISVDKSKNQFSLKLSSVTAADTAVYYCARSTWSLDYWGQGTLVTVSS

SEQ ID NO: 278 - protein, 112 aa, Artificial Sequence; identical to SEQ ID NO: 206
  light chain variable region L39

SEQ ID NO: 279 - protein, 118 aa, Artificial Sequence
  Synthetic Polypeptide
  MISC_FEATURE: heavy chain variable region H39
EVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCARLSFADPFDIWGQGTMVTVSS

SEQ ID NO: 280 - protein, 107 aa, Artificial Sequence
  Synthetic Polypeptide
  MISC_FEATURE: light chain variable region L40
ETTLTQSPATLSLSPGQRATLSCRASQSVYNYLAWYQQKPGQAPRLLIYDASRRATGIPARFSGSGSGTDFTLTISSLEPEDFAVYYCQQRNNWPLTFGGGTKVEIK

SEQ ID NO: 281 - protein, 122 aa, Artificial Sequence
  Synthetic Polypeptide
  MISC_FEATURE: heavy chain variable region H40
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAYGSGSYYDYYYMDVWGKGTTVTVSS

SEQ ID NO: 282 - protein, 107 aa, Artificial Sequence
  Synthetic Polypeptide
  MISC_FEATURE: light chain variable region L41
DIQLTQSPSSLSASVGDSVTISCRASQSPGIFLNWYQQIPGKAPKLLIYATSTLESGVPPRFTGSGSGTDFTLTISSLQPEDFATYYCQQSNSVPLTFGGGTKVEIK

SEQ ID NO: 283 - protein, 119 aa, Artificial Sequence
  Synthetic Polypeptide
  MISC_FEATURE: heavy chain variable region H41
EVQLVQSGGGLVQPGGSLRLSCSASGFTFSSYAMHWVRQAPGKGLEYVSTISSNGDSTYYADSVKGRFTISRDNSKNTLYLQMNSLRAEDTAVYYCAKEEVWLQAFDIWGQGTMVTVSS

SEQ ID NO: 284 - protein, 112 aa, Artificial Sequence
  Synthetic Polypeptide
  MISC_FEATURE: light chain variable region L42
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGQGTRLEIK

SEQ ID NO: 285 - protein, 115 aa, Artificial Sequence
  Synthetic Polypeptide
  MISC_FEATURE: heavy chain variable region H42
QLQLQESGPGLVKPSETLSLTCTVSGGSISSNWWSWVRQPPGKGLEWIGEIYHSGSTNYNPSLKSRVTISVDTSKNQFSLKLSSVTAADTAVYYCARDKGYMDVWGKGTTVTVSS

SEQ ID NO: 286 - protein, 107 aa, Artificial Sequence
  Synthetic Polypeptide
  MISC_FEATURE: light chain variable region L43
EIVMTQSPATLSVSPGERATFSCRASQSVGSNLAWYQQKPGQAPRLLIYDASNRATGIPARFSGSGSGTDFTLTISRLEPEDFAVYYCQQRSNWPLTFGGGTKVEIK

SEQ ID NO: 287 - protein, 121 aa, Artificial Sequence
  Synthetic Polypeptide
  MISC_FEATURE: heavy chain variable region H43
QVQLQQSGAEVKKPGSSVKVSCKASGGTFSSYAISWVRQAPGQGLEWMGRIIPILGIANYAQKFQGRVTITADKSTSTAYMELSSLRSEDTAVYYCARDHRFDYAWYFDLWGRGTLVTVSS

SEQ ID NO: 288 - protein, 112 aa, Artificial Sequence; identical to SEQ ID NO: 206
  Synthetic Polypeptide
  MISC_FEATURE: light chain variable region L44

SEQ ID NO: 289 - protein, 117 aa, Artificial Sequence
  Synthetic Polypeptide
  MISC_FEATURE: heavy chain variable region H44
QVQLQESGPGLLKPSGTLSLTCAVSGGSISSSNWWSWVRQPPGEGLEWIGEIYHSGSTNYNPSLKSRVTISVDKSKNQFSLKLSSVTAADTAVYYCARDLTGSLDYWGQGTLVTVSS

SEQ ID NO: 290 - protein, 112 aa, Artificial Sequence
  Synthetic Polypeptide
  MISC_FEATURE: light chain variable region L45
DVVMTQSPLSLPVTPGEPASISCRSSQSLLHSNGYNYLDWYLQKPGQSPQLLIYLGSTRASGVPDRFSGSGSGTDFTLKISRVEAEDVGVYYCMQALQTPYTFGGGTKVEIK

SEQ ID NO: 291 - protein, 117 aa, Artificial Sequence
  Synthetic Polypeptide
  MISC_FEATURE: heavy chain variable region H45
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARIRYDAFDIWGQGTMVTVSS

SEQ ID NO: 292 - protein, 112 aa, Artificial Sequence; identical to SEQ ID NO: 206
  Synthetic Polypeptide
  MISC_FEATURE: light chain variable region L46

SEQ ID NO: 293 - protein, 118 aa, Artificial Sequence
  Synthetic Polypeptide
  MISC_FEATURE: heavy chain variable region H46
QVQLQESGPGLVKPSETLSLTCAVSGGSISSSNWWSWVRQPPGKGLEWIGEIYHSGSTNYNPSLKSRVTISVDKSKNQFSLKLSSVTAADTAVYYCAVTAAHDAFDIWGQGTMVTVSS

SEQ ID NO: 294 - protein, 112 aa, Artificial Sequence
  Synthetic Polypeptide
  MISC_FEATURE: light chain variable region L47
DVVMTQSPLSLPVTPGEPASISCRSSQSLLHTNGYNYLDWYLQKPGQSPRLLIYLGFNRASGVPDRFSGSGSGTDFTLKISRVEAEDVGVYYCMQGLQTPLTFGGGTKVEIK

SEQ ID NO: 295 - protein, 119 aa, Artificial Sequence
  Synthetic Polypeptide
  MISC_FEATURE: heavy chain variable region H47
QVQLQQWGPGLVKPSGTLSLTCAVSGGSISSSNWWSWVRQPPGKGLEWIGEIYHSGSTNYNPSLKSRVTISVDKSKNQFSLKLSSVTAADTAVYYCARDSSGQGYFDYWGQGTLVTVSS

SEQ ID NO: 296 - protein, 112 aa, Artificial Sequence
  Synthetic Polypeptide
  MISC_FEATURE: light chain variable region L48
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQATHWPYTFGQGTKLEIK

SEQ ID NO: 297 - protein, 118 aa, Artificial Sequence
  Synthetic Polypeptide
  MISC_FEATURE: heavy chain variable region H48
EVQLVQSGAEVKKPGASVKVSCKASGYTFTSYAMHWVRQAPGQRLEWMGWINAGNGNTKYSQKFQGRVTMTRDTSTSTVYMELSSLRSEDTAVYYCARHSYYYGMDVWGQGTLVTVSS

SEQ ID NO: 298 - protein, 110 aa, Artificial Sequence
  Synthetic Polypeptide
  MISC_FEATURE: light chain variable region L49
NFMLTQPHSVSESPGKTVSISCTRNSGSIASNFVQWYQQRPGSAPTIVIYEDNQRPSAVPTRFSGSIDRSSNSASLTISGLTTEDEADYYCQSYDSANVIFGGGTKLTVL

SEQ ID NO: 299 - protein, 120 aa, Artificial Sequence
  Synthetic Polypeptide
  MISC_FEATURE: heavy chain variable region H49
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVGYSHGEEVLDVWGKGTTVTVSS

SEQ ID NO: 300 - protein, 108 aa, Artificial Sequence
  Synthetic Polypeptide
  MISC_FEATURE: light chain variable region L50
ETTLTQSPGTLSLSPGERATLSCRASQTISSSHLAWYQQKPGQSPRLLIYGAGYRATGIPDRFSGSGSGTDFTLTISRLEPEDFAVYYCQHYGSSLRTFGQGTKVEIK

SEQ ID NO: 301 - protein, 118 aa, Artificial Sequence
  Synthetic Polypeptide
  MISC_FEATURE: heavy chain variable region H50
QVQLQESGPGLVKPSETLSLTCTVSGGSIGNYDWSWIRQPPGKGLEWIGTIYSSGSTYYSPSLKSRLTISVDKSKNRFSLKLSSVTAADTAVYYCARARGYSSPFDPWGQGTLVTVSS

SEQ ID NO: 302 - protein, 110 aa, Artificial Sequence
  Synthetic Polypeptid
  MISC_FEATURE: light chain variable region L51
NFMLTQPHSVSESPGKTVTISCTGSGGNIASNYVQWYQQRPGRAPTTVIYEDNRRPSGVPDRFSGSIDSSSNSASLTISGLKTEDEADYYCQSYDPYNRVFGGGTKLTVL

SEQ ID NO: 303 - protein, 119 aa, Artificial Sequence
  Synthetic Polypeptide
  MISC_FEATURE: heavy chain variable region H51
QVQLVQSGAEVKKPGSSVKVSCKASGGTFSSYAISWVRQAPGQGLEWMGIINPSGGSTSYAQKFQGRVTITRDTSASTAYMELSSLRSEDTAVYYCARDRWRYDAFDIWGQGTMVTVSS

SEQ ID NO: 304 - protein, 112 aa, Artificial Sequence
  Synthetic Polypeptide
  MISC_FEATURE: light chain variable region L52
EIVMTQSPLSLPVTPGEPASISCRSSQSLLHTNGYDYLDWYLQKPGQSPQLLIYLGSTRASGVPDRFSGSGSGTDFTLKISRVEAEDVGVYYCMQAFQTPLTFGGGTKMEIK

SEQ ID NO: 305 - protein, 116 aa, Artificial Sequence
  Synthetic Polypeptide
  MISC_FEATURE: heavy chain variable region H52
EVQLVESGPGLVKPSGTLSLTCAVSGGSISSSNWWSWVRQPPGKGLEWIGEIYHSGSTNYNPSLKSRVTISVDKSKNQFSLKLSSVTAADTAVYYCAREKSGMDVWGQGTTVTVSS

SEQ ID NO: 306 - protein, 16 aa, Artificial Sequence
  Synthetic Polypeptide
  MISC_FEATURE: AB16 LC CDR1
RSSQSLLHSNGYNYLD

SEQ ID NO: 307 - protein, 7 aa, Artificial Sequence
  Synthetic Polypeptide
  MISC_FEATURE: AB16 LC CDR2
LGSNRAS

SEQ ID NO: 308 - protein, 9 aa, Artificial Sequence
  Synthetic Polypeptide
  MISC_FEATURE: AB16 LC CDR3
MQGTHWPLT

SEQ ID NO: 309 - protein, 6 aa, Artificial Sequence
  Synthetic Polypeptide
  MISC_FEATURE: AB16 HC CDR1
SSNWWS

SEQ ID NO: 310 - protein, 16 aa, Artificial Sequence
  Synthetic Polypeptide
  MISC_FEATURE: AB16 HC CDR2
EIYHSGSTNYNPSLKS

SEQ ID NO: 311 - protein, 10 aa, Artificial Sequence
  Synthetic Polypeptide
  MISC_FEATURE: AB16 HC CDR3
WTGRTDAFDI

SEQ ID NO: 312 - protein, 9 aa, Artificial Sequence
  Synthetic Polypeptide
  MISC_FEATURE: AB24 LC CDR3
MQALQTPNT

SEQ ID NO: 313 - protein, 7 aa, Artificial Sequence
  Synthetic Polypeptide
  MISC_FEATURE: AB24 HC CDR1
SSSYYWG

SEQ ID NO: 314 - protein, 16 aa, Artificial Sequence
  Synthetic Polypeptide
  MISC_FEATURE: AB24 HC CDR2
SIYYSGSTYYNPSLKS

SEQ ID NO: 315 - protein, 11 aa, Artificial Sequence
  Synthetic Polypeptide
  MISC_FEATURE: AB24 HC CDR3
DGGYYYYGMDV

SEQ ID NO: 316 - protein, 13 aa, Artificial Sequence
  Synthetic Polypeptide
  MISC_FEATURE: AB51 LC CDR1
TGSGGNIASNYVQ

SEQ ID NO: 317 - protein, 7 aa, Artificial Sequence
  Synthetic Polypeptide
  MISC_FEATURE: AB51 LC CDR2
EDNRRPS

SEQ ID NO: 318 - protein, 9 aa, Artificial Sequence
  Synthetic Polypeptide
  MISC_FEATURE: AB51 LC CDR3
QSYDPYNRV

SEQ ID NO: 319 - protein, 5 aa, Artificial Sequence
  Synthetic Polypeptide
  MISC_FEATURE: AB51 HC CDR1
SYAIS

SEQ ID NO: 320 - protein, 17 aa, Artificial Sequence
  Synthetic Polypeptide
  MISC_FEATURE: AB51 HC CDR2
IINPSGGSTSYAQKFQG

SEQ ID NO: 321 - protein, 10 aa, Artificial Sequence
  Synthetic Polypeptide
  MISC_FEATURE: AB51 HC CDR3
DRWRYDAFDI

SEQ ID NO: 322 - protein, 16 aa, Artificial Sequence
  Synthetic Polypeptide
  MISC_FEATURE: AB52 LC CDR1
RSSQSLLHTNGYDYLD

SEQ ID NO: 323 - protein, 7 aa, Artificial Sequence
  Synthetic Polypeptide
  MISC_FEATURE: AB52 LC CDR2
LGSTRAS

SEQ ID NO: 324 - protein, 9 aa, Artificial Sequence
  Synthetic Polypeptide
  MISC_FEATURE: AB52 LC CDR3
MQAFQTPLT

SEQ ID NO: 325 - protein, 6 aa, Artificial Sequence
  Synthetic Polypeptide
  MISC_FEATURE: AB52 HC CDR1
SSNWWS

SEQ ID NO: 326 - protein, 16 aa, Artificial Sequence
  Synthetic Polypeptide
  MISC_FEATURE: AB52 HC CDR2
EIYHSGSTNYNPSLKS

SEQ ID NO: 327 - protein, 7 aa, Artificial Sequence
  Synthetic Polypeptide
  MISC_FEATURE: AB52 HC CDR3
EKSGMDV